(19) United States
(12) Patent Application Publication
(10) Pub. No.: US 2015/0057574 A1
(43) Pub. Date: Feb. 26, 2015

(54) Selecting and Delivering Treatment Agents based on a Microbe Profile

(71) Applicant: Elwha LLC, Bellevue, WA (US)

(72) Inventors: Michael H. Baym, Cambridge, MA (US); Roderick A. Hyde, Redmond, WA (US); Jordin T. Kare, Seattle, WA (US); Eric C. Leuthardt, St. Louis, MO (US); Gary L. McKnight, Bothell, WA (US); Tony S. Pan, Bellevue, WA (US); Elizabeth A. Sweeney, Seattle, WA (US); Clarence T. Tegreene, Mercer Island, WA (US); Lowell L. Wood, Jr., Bellevue, WA (US)

(21) Appl. No.: 14/255,653

(22) Filed: Apr. 17, 2014

Related U.S. Application Data
(63) Continuation-in-part of application No. 13/975,055, filed on Aug. 23, 2013.
Application No. 13/975,055 is a continuation-in-part of application No. 14/091,762, filed on Nov. 27, 2013.
Application No. 14/091,762 is a continuation-in-part of application No. 14/091,832, filed on Nov. 27, 2013.
Application No. 14/091,832 is a continuation-in-part of application No. 14/192,613, filed on Feb. 27, 2014.

(51) Int. Cl. A61M 5/172; A61B 10/00

(52) U.S. Cl. CPC A61M 5/1723; A61B 10/00
USPC 600/570; 600/562; 600/572; 600/569; 604/289

(57) ABSTRACT

Kits, devices, methods, and systems for assessing microbiota of a body surface and selecting treatment agents to apply to the body surface to modulate one or more types of microbes are described which include a kit including a plurality of treatment agents, a microbe sampling unit including a microbe-capture region, and an analyzer including a user interface, at least one sensor component, and a computing component with a processor and circuitry.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

The present application constitutes a continuation-in-part of U.S. patent application Ser. No. 13/975,055, entitled SYSTEMS, METHODS, AND DEVICES FOR ASSESSING MICROBIOTA OF SKIN, naming Mahalaxmi G. Bangera, Michael H. Baym, Roderick A. Hyde, Jordin T. Kare, Eric C. Leuthardt, Gary L. McKnight, Tony S. Pan, Katherine E. Sharadin, Elizabeth A. Sweeney, Clarence T. Tegreene, and Lowell L. Wood, Jr. as inventors, filed 23, Aug., 2013 with attorney docket no. 0912-002-005-000000, which is currently co-pending or is an application of which a currently co-pending application is entitled to the benefit of the filing date.
The present application constitutes a continuation-in-part of U.S. patent application Ser. No. 14/091,762, entitled DEVICES AND METHODS FOR PROFILING MICROBIOTA OF SKIN, naming Michael H. Baym, Roderick A. Hyde, Jordin T. Kare, Eric C. Leuthardt, Gary L. McKnight, Tony S. Pan, Elizabeth A. Sweeney, Clarence T. Tegreene, Lowell L. Wood, Jr. as inventors, filed 27, Nov., 2013 with attorney docket no. 0912-002-011-000000, which is currently co-pending or is an application of which a currently co-pending application is entitled to the benefit of the filing date.
The present application constitutes a continuation-in-part of U.S. patent application Ser. No. 14/091,832, entitled DEVICES AND METHODS FOR SAMPLING AND PROFILING MICROBIOTA OF SKIN, naming Michael H. Baym, Roderick A. Hyde, Jordin T. Kare, Eric C. Leuthardt, Gary L. McKnight, Tony S. Pan, Elizabeth A. Sweeney, Clarence T. Tegreene, Lowell L. Wood, Jr. as inventors, filed 27, Nov., 2013 with attorney docket no. 0912-002-014-000000, which is currently co-pending or is an application of which a currently co-pending application is entitled to the benefit of the filing date.
The present application constitutes a continuation-in-part of U.S. patent application Ser. No. 14/192,613 entitled SYSTEMS, METHODS, AND DEVICES FOR DELIVERING TREATMENT TO A SKIN SURFACE, naming Roderick A. Hyde and Gary L. McKnight as inventors, filed 27, Feb., 2014 with attorney docket no. 0912-002-016-000000, which is currently co-pending or is an application of which a currently co-pending application is entitled to the benefit of the filing date.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a kit includes, but is not limited to, a plurality of treatment agents; a microbe sampling unit having at least one surface with a microbe-capture region, the microbe-capture region configured to capture one or more types of microbes from a body surface of a user; and an analyzer including at least one sensor component including circuitry to detect one or more signals emitted or reflected from the microbe-capture region of the microbe sampling unit and to transform the detected one or more signals into a sensor output; a user interface; and a computing component including a processor and operably coupled to the at least one sensor component and the user interface, the computing component including circuitry configured to receive the sensor output from the at least one sensor component, the sensor output including information associated with at least one property of the detected one or more signals emitted or reflected from the microbe-capture region of the microbe sampling unit; compare the at least one property of the detected one or more signals emitted or reflected from the microbe-capture region of the microbe sampling unit with a reference dataset of signal properties; generate a microbe profile of the user based on the comparison with the reference dataset of signal properties, compare the microbe profile of the user with at least one reference microbe profile; and recommend to the user at least one of the plurality of treatment agents based on the comparison with the at least one reference microbe profile. In addition to the foregoing, other kit aspects are described in the claims, drawings, and text forming a part of the present disclosure.

In an aspect, a kiosk includes, but is not limited to, a plurality of treatment agents; one or more dispensers to dispense at least one of the plurality of treatment agents; at least one microbe sampling unit, the at least one microbe sampling unit including at least one surface with a microbe-capture region, the microbe-capture region configured to capture one or more types of microbes from a body surface of a user; a user interface; at least one sensor component including circuitry to detect one or more signals emitted or reflected from the microbe-capture region of the at least one microbe sampling unit and to transform the detected one or more signals into a signal output; and a computing component including a processor and operably coupled to the one or more dispensers, the at least one sensor component, and the user interface, the computing component including circuitry configured to receive the sensor output from the at least one sensor component, the sensor output including information associated with at least one property of the detected one or more signals emitted or reflected from the microbe-capture region of the at least one microbe sampling unit; compare the at least one property of the detected one or more signals emitted or reflected from the microbe-capture region of the at least one microbe sampling unit with a reference dataset of signal properties; generate a microbe profile of the user based on the comparison with the reference dataset of signal properties; compare the microbe profile of the user with at least one reference microbe profile; recommend at least one of the plurality of treatment agents to the user based on the comparison with the at least one reference microbe profile; and send a signal to at least one of the one or more dispensers to dispense the recommended at least one of the plurality of treatment agents from the kiosk to the user. In addition to the foregoing, other aspects of a kiosk are described in the claims, drawings, and text forming a part of the present disclosure.

In an aspect, a method includes, but is not limited to, receiving information associated with a microbe profile of an individual from a remote source, the microbe profile including a distribution of one or more types of microbes on a body surface of the individual; selecting one or more treatment agents from a list of available treatment agents to apply to the body surface to modulate the one or more types of microbes on the body surface of the individual; and arranging for delivery of the selected one or more treatment agents. In addition to the foregoing, other aspects of a method are described in the claims, drawings, and text forming a part of the present disclosure.

In an aspect, a system includes, but is not limited to, circuitry for receiving information associated with a microbe profile of an individual from a remote source, the microbe profile including a distribution of one or more types of microbes on a body surface of the individual; circuitry for selecting one or more treatment agents from a list of available treatment agents to apply to the body surface to modulate the one or more types of microbes on the body surface of the individual; and circuitry for arranging for delivery of the selected one or more treatment agents. In addition to the foregoing, other aspects of a system are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of a kit.

FIG. 2A is a schematic of a kit including a mask.

FIG. 2B illustrates a mask of a kit on a user.

FIG. 2C illustrates analyzing a mask of a kit with an analyzer of the kit.

FIG. 3 illustrates further aspects of a kit.

FIG. 4A is an embodiment of a microbe sampling unit of a kit.

FIG. 4B is an embodiment of a microbe sampling unit of a kit.

FIG. 4C is an embodiment of a microbe sampling unit of a kit.

FIG. 4D is an embodiment of a microbe sampling unit of a kit.

FIG. 4E is an embodiment of a microbe sampling unit of a kit.

FIG. 4F is an embodiment of a microbe sampling unit of a kit.

FIG. 4G is an embodiment of a microbe sampling unit of a kit.

FIG. 4H is an embodiment of a microbe sampling unit of a kit.

FIG. 4I is an embodiment of a microbe sampling unit of a kit.

FIG. 4J is an embodiment of a microbe sampling unit of a kit.

FIG. 5A is a schematic of a microbe-capture region

FIG. 5B is a schematic of a microbe-capture region with one or more types of microbes.

FIG. 6A is a schematic of a microbe-capture region with a plurality of specific microbe-binding elements.

FIG. 6B is a schematic of a microbe-capture region with a plurality of specific microbe-binding elements with a specific type of microbe.

FIG. 7A is a schematic of a microbe-capture region with a plurality of specific microbe-binding elements of a first type and a second type.

FIG. 7B is a schematic of a microbe-capture region with a plurality of specific microbe-binding elements of a first type and a second type with microbes of a first and second type.

FIG. 8A is a schematic of a microbe-capture region with one or more types of microbes and a plurality of signal-generating elements.

FIG. 8B is a schematic of a microbe-capture region with a plurality of specific microbe-binding elements with a specific type of microbe and a plurality of signal-generating elements.

FIG. 9A is a schematic of a microbe-capture region with a plurality of signal-generating complexes in a first state.

FIG. 9B is a schematic of a microbe-capture region with a specific type of microbe and a plurality of signal-generating complexes in a second state.

FIG. 10 illustrates further aspects of a kit such as shown in FIG. 1.

FIG. 11 shows further aspects of a kit such as depicted in FIG. 1.

FIG. 12 depicts further aspects of a kit such as illustrated in FIG. 1.

FIG. 13 illustrates further aspects of a kit such as shown in FIG. 1.

FIG. 14 shows further aspects of a kit such as depicted in FIG. 1.

FIG. 15 is a schematic of a kiosk.

FIG. 16 is a schematic of a kiosk with a user.

FIG. 17 illustrates further aspects of a kiosk such as shown in FIG. 15.

FIG. 18 shows further aspects of a kiosk such as depicted in FIG. 15.

FIG. 19 depicts further aspects of a kiosk such as illustrated in FIG. 15.

FIG. 20 illustrates further aspects of a kiosk such as shown in FIG. 15.

FIG. 21 shows further aspects of a kiosk such as depicted in FIG. 15.

FIG. 22 depicts further aspects of a kiosk such as illustrated in FIG. 15.

FIG. 23 illustrates further aspects of a kiosk such as shown in FIG. 15.

FIG. 24 is a flowchart of a method of selecting and arranging for delivery of one or more treatment agents.

FIG. 25 illustrates further aspects of a method such as shown in FIG. 24.

FIG. 26 shows further aspects of a method such as depicted in FIG. 24.

FIG. 27 depicts further aspects of a method such as illustrated in FIG. 24.

FIG. 28 is a schematic of a system including circuitry.

FIG. 29 is a schematic of a system including circuitry and a computing device

FIG. 30 shows further aspects of a system such as depicted in FIG. 28.

FIG. 31 depicts further aspects of a system such as illustrated in FIG. 28.

FIG. 32 illustrates further aspects of a system such as shown in FIG. 28.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Microbiota, the collective microbial community inhabiting a specific environment, e.g., a human body, can include archaea, bacteria, viruses, and eukaryotes. The microbes comprising the microbiota can play important roles in the health of a body including metabolism, homeostasis of the immune system, pathogenesis, and colonization resistance. The skin, the largest organ of the mammalian body, is inhabited by a diverse array of microbes, including bacteria, fungi, viruses, parasites, archaea, or small arthropods (e.g., mites). Variations in regional properties of the skin, e.g., variations in pH, moisture, pores, texture, and the like, from one body location to another contribute to the spatial diversity of skin-associated microbes. Similarly, the type of microbes and/or spatial distribution of one or more microbes on the skin surface may change in response to cleaning of the skin surface, application of anti-microbial agents, application of irritating agents, e.g., make-up, lotion, sun screen, or exposure to irritating conditions, e.g., diet, disease, wind, or sun exposure. In some instances, skin-resident microbes on the skin surface, e.g., commensal bacteria, provide a benefit to the individual. For example, Staphylococcus epidermidis has been demonstrated to modulate the host innate immune response, inhibiting other bacterial pathogens such as Staphylococcus aureus and Group A Streptococcus. See, e.g., Grice & Segre (2011) Nat. Rev. Microbiol. 9:244-53, which is incorporated herein by reference. In some instances, skin-resident microbes have been linked to pathological conditions including acne, psoriasis, and atopic dermatitis. See, e.g., Cho & Blaser (2012) Nat. Rev. Genet. 13:260-270, which is incorporated herein by reference. In general, understanding the identity and spatial distribution of skin-resident microbes on the skin under normal and/or pathological conditions can contribute to decisions regarding therapeutic, preventative, and/or cosmetic treatments. Described here are embodiments of devices, kits, methods, and systems for assessing microbiota of a body surface, generating a microbe profile, selecting treatment agents from a selection of treatment agents based on the microbe profile, and delivering the selected treatment agents to a user.

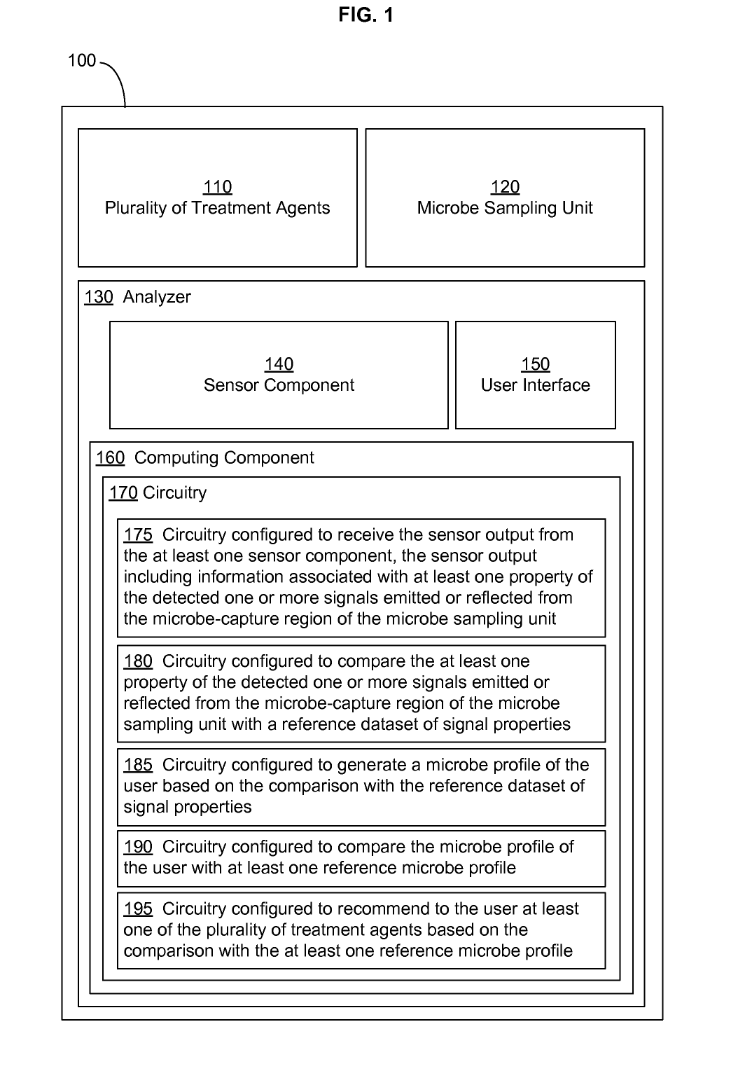

With reference to FIG. 1, shown is an example of a kit including components configured to sample one or more types of microbes from a body surface of a user, generate a microbe profile, and recommend one or more treatment agents included in the kit. Kit 100 includes plurality of treatment agents 110, microbe sampling unit 120, and analyzer 130. Plurality of treatment agents 110 includes one or more agents for application to a body surface of the user to modulate one or more types of microbes on said body surface. Microbe sampling unit 120 has at least one surface with a microbe-capture region, the microbe-capture region configured to capture one or more types of microbes from a body surface of a user. Analyzer 130 includes at least one sensor component 140, user interface 150, and computing component 160. At least one sensor component 140 includes circuitry configured to detect one or more signals emitted or reflected from the microbe-capture region of the microbe sampling unit 120 and to transform the detected one or more signals into a sensor output. User interface 150 includes one or more components for input and output of information. Computing component 160 includes a processor, e.g., a microprocessor, and is operably coupled to at least one sensor component 140 and user interface 150. Computing component 160 further includes circuitry 170. Circuitry 170 includes circuitry 175 configured to receive the sensor output from the at least one sensor component 140, the sensor output including information associated with at least one property of the detected one or more signals emitted or reflected from the microbe-capture region of microbe sampling unit 120; circuitry 180 configured to compare the at least one property of the detected one or more signals emitted or reflected from the microbe-capture region of microbe sampling unit 120 with a reference dataset of signal properties; circuitry 185 configured to generate a microbe profile of the user based on the comparison with the reference dataset of signal properties; circuitry 190 configured to compare the microbe profile of the user with at least one reference microbe profile; and circuitry 195 configured to recommend to the user at least one of the plurality of treatment agents 110 based on the comparison with the at least one reference microbe profile.

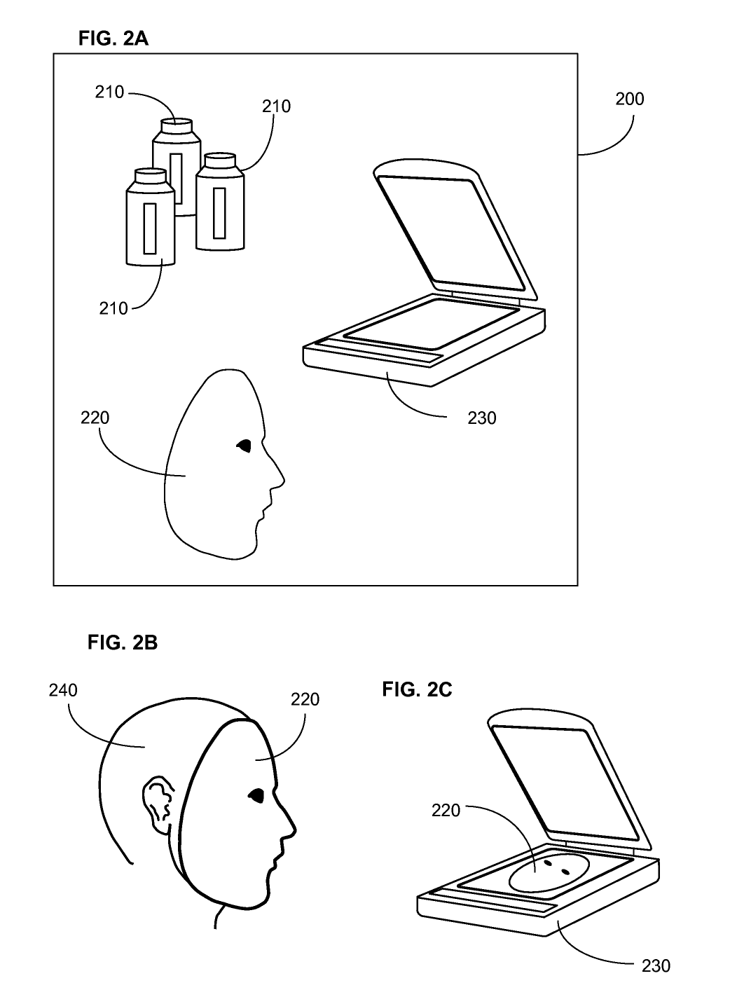

FIG. 2A shows an embodiment of a kit such as described in FIG. 1. Kit 200 includes a plurality of treatment agents 210, e.g., two or more containers, each container including one or more treatment agents for application to a body surface of a user to modulate one or more types of microbes on said body surface, e.g., a skin surface, of the user. Kit 200 further includes microbe sampling unit 220, e.g., a pre-formed or peelable mask, including at least one surface with a microbe-capture region. In an aspect, microbe sampling unit 220 substantially conforms to a topography of the body surface of the user, e.g., to the topography of the skin surface of the user. Kit 200 further includes analyzer 230, e.g., an optical scanning device, including at least one sensor component, a user interface, and a computing component. FIG. 2B shows microbe sampling unit 220, e.g., a mask, in contact with the skin surface of user 240. FIG. 2C shows microbe sampling unit 220 on a surface, e.g., a scanning surface, of analyzer 230 in preparation for detecting one or more signals emitted or reflected from the microbe-capture region of microbe sampling unit 220. Analyzer 230 further includes a computing component including circuitry configured to receive a sensor output from the at least one sensor component, the sensor output including information associated with at least one property of the detected one or more signals emitted or reflected from the microbe-capture region of microbe sampling unit 220; circuitry configured to compare the at least one property of the detected one or more signals emitted or reflected from the microbe-capture region of microbe sampling unit 220 with a reference dataset of signal properties; circuitry configured to generate a microbe profile of user 240 based on the comparison with the reference dataset of signal properties; circuitry configured to compare the microbe profile of user 240 with at least one reference microbe profile; and circuitry configured to recommend to user 240 at least one of the plurality of treatment agents 210 based on the comparison with the at least one reference microbe profile.

### Treatment Agents

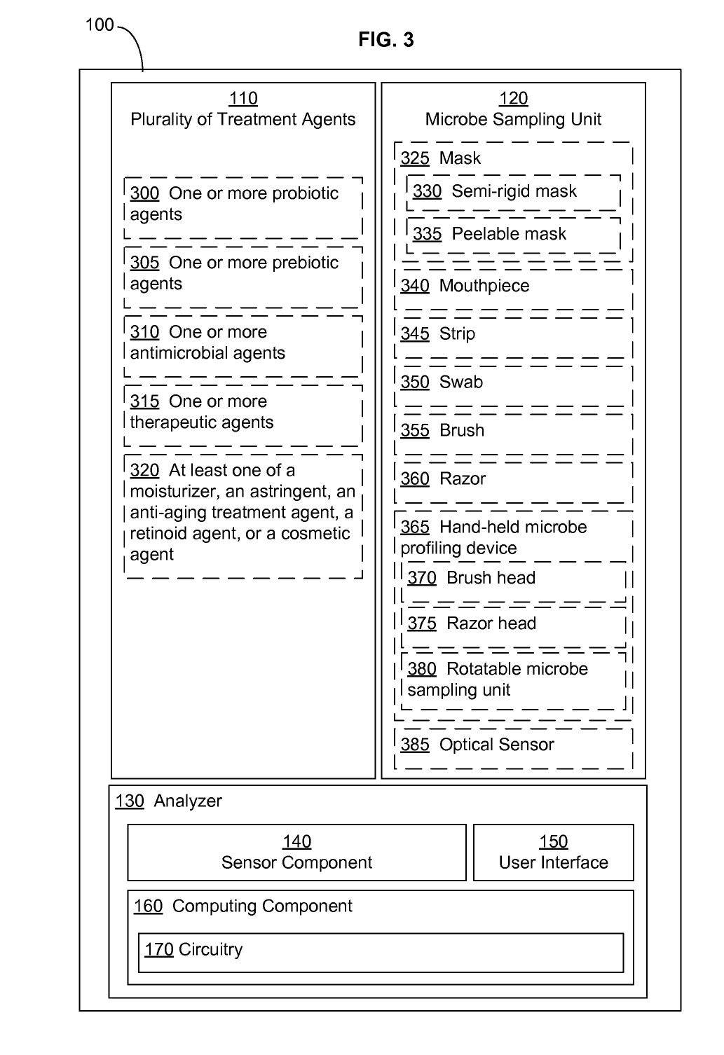

FIG. 3 illustrates further aspects of a kit such as shown in FIG. 1. Kit 100 includes plurality of treatment agents 110. In an aspect, the plurality of treatment agents includes two or more treatment agents for application to a body surface to modulate one or more types of microbes on said body surface of a user. In an aspect, the plurality of treatment agents includes two or more treatment agents for application to a skin surface to modulate one or more types of microbes on the skin surface of a user. It should be understood that this disclosure refers to skin by example only but is also intended to include other body surfaces including but not limited to a gastrointestinal surface, a mucosal surface, a vaginal surface, a nasal surface, or an oral surface of a user. In an aspect, the plurality of treatment agents includes two or more treatment agents for application to a gastrointestinal surface, mucosal surface, a vaginal surface, a nasal surface, and/or an oral surface to modulate one or more types of microbe on said body surface of the user. In an aspect, to modulate one or more types of microbes includes inhibiting the growth of one or more types of microbes. In an aspect, to modulate one or more types of microbe includes promoting the growth of one or more types of microbes. In an aspect, to modulate one or more types of microbes includes preventing the attachment of one or more types of microbes. In an aspect, to modulate one or more types of microbes includes preventing the growth of one or more types of microbes. In an aspect, to modulate one or more types of microbes includes preventing the colonization of one or more types of microbes. In an aspect, at least one of the plurality of treatment agents includes at least one agent to modulate an environment. In an aspect, at least one of the plurality of treatment agents includes at least one agent to modulate an environment, e.g., to influence one or more types of microbes. In an aspect, at least one of the plurality of treatment agents includes at least one agent to modulate an environment, e.g., the pH of a body surface, to prevent, inhibit, or promote the attachment, growth, or colonization of one or more types of microbes.

In an aspect, the kit includes two or more containers, each container including one or more treatment agents. In an aspect, each container includes a single treatment agent. In an aspect, each container includes a combination of treatment agents. In an aspect, the plurality of treatment agents includes at least one first treatment agent in at least one first container and at least one second treatment agent in at least one second container. In an aspect, the plurality of treatment agents includes at least one third treatment agent in at least one third container. In an aspect, the plurality of treatment agents includes at least one or more additional treatment agents in at least one or more additional containers. In an aspect, each container is labeled, e.g., with an alphanumeric code or bar code. In an aspect, the kit further includes at least one empty container for use in mixing at least a portion of the contents of two or more other containers including one or more treatment agents to generate a mixture of treatment agents for application to a body surface of a user.

In an aspect, the plurality of treatment agents include one or more agents configured to maintain, alter, and/or improve the microbiota, e.g., the types and quantity of microorganisms, on a body surface of a user, e.g., a skin, gastrointestinal, mucosal, vaginal, nasal, or oral surface of the user. In an aspect, the one or more treatment agents include one or more agents configured to treat a condition or disease on the body surface of the user. In an aspect, the one or more treatment agents include one or more agents configured to prevent a condition or disease on the body surface of the user.

In an aspect, the one or more treatment agents include one or more agents configured to treat a condition or disease on the skin surface of the user. Non-limiting examples of conditions or diseases of the skin include inflammatory or immune-related disorders, e.g., eczema, hives, atopic dermatitis, or psoriasis; a microbial infection, e.g., a bacterial, fungal, or viral infection; acne, actinic keratosis, rosacea, seborrheic dermatitis, seborrheic keratosis, warts, or skin cancer, e.g., melanoma, squamous cell carcinoma, or basal cell carcinoma; tinea pedis; aging skin; and dry or sensitive skin.

In an aspect, the one or more treatment agents include one or more agents configured to treat a condition or disease of the gastrointestinal tract. Non-limiting examples of conditions or diseases of the gastrointestinal tract include microbial infection, e.g., bacterial, fungal, or viral infection, and/or inflammatory conditions, e.g., gastritis, inflammatory bowel disease, enterocolitis. In an aspect, the one or more treatment agents include one or more agents configured to treat a condition or disease of a mucosal region, a vaginal region, a nasal region, or a mouth region.

Referring again to FIG. 3, in an aspect, the plurality of treatment agents 110 includes one or more probiotic agents 300. In an aspect, the one or more probiotic agents include one or more commensal microorganisms which positively affect microbiota of a body surface, e.g., a skin, gastrointestinal, mucosal, vaginal, nasal, or oral surface. In an aspect, the one or more probiotic agents include one or more skin commensal microorganisms which positively affect the skin microbiota. For example, the one or more probiotic agents can include microorganisms that positively affect the skin surface environment, e.g., by altering the pH or inhibiting growth of pathogenic microorganisms. In an aspect, the one or more probiotic agents can include one or more microorganisms naturally found on the body surface, e.g., skin surface, of the individual. In an aspect, the one or more probiotic agents can include one or more microorganism that are not naturally found on the body surface, e.g., skin surface, of the individual, but positively affect the body surface environment. In an aspect, the one or more probiotic agents can include one or more engineered microorganisms. For example, the one or more probiotic agents can include a microorganism genetically engineered to have a property that positively affects the body surface environment, e.g., by synthesizing and excreting an inhibitor of pathogenic microorganisms. See, e.g., Martin et al. (2013) Microbial Cell Factories, 12:71, which is incorporated herein by reference. In an aspect, the one or more probiotic agents comprise live probiotic microorganisms. In an aspect, the one or more probiotic agents may be included in a live form, dead form, semi-active or deactivated form, and/or in fragments or fractions originating from the microorganism either live or dead (e.g., as a lyophilized powder). In an aspect, the one or more probiotic agents include culture supernatants of the microorganisms. In an aspect, the one or more probiotic agents include one or more microorganisms which positively affect the microbiota of the skin, gastrointestinal tract, mucosa, vagina, nasal cavity, or oral region. See, e.g., Hardy et al. (2013) Nutrients 5:1869-1912; Hemmerling et al. (2009) Sex Transm Dis 36:564-569; and Belkaid et al. (2013) Nat Immunol 14:646-53, each of which is incorporated herein by reference.

In an aspect, the one or more probiotic agents include one or more bacterial probiotics. See, e.g., U.S. Pat. No. 8,557,560, U.S. Patent Applications 2011/0274676, 2014/0037688, Schrezenmeir & De Vrese (2001) Am. J. Clin. Nutr. 73(suppl):361S-364S, and Gueniche et al. (2009) Exp. Dermatol. 19:e1-e8, which are incorporated herein by reference. In an aspect, the one or more bacterial probiotic agents include at least one type of bacteria from Firmicutes, Actinobacteria, Bacteriodetes, Proteobacteria, or Cyanobacteria. In an aspect, the one or more bacterial probiotic agents include at least one type of bacteria from Corynebacteria, Propionibacteria, Micrococci, or Staphylococci. In an aspect, the one or more bacterial probiotics include non-lactic acid and/or lactic acid producing bacteria (LAB) and can include Bacteroides, Bifidobacterium, and Lactobacillus. In an aspect, the one or more bacterial probiotics include certain strains of Aerococcus, E. coli, Bacillus, Enterococcus, Fusobacterium, Lactococcus, Leuconostoc, Melissacoccus, Micrococcus, Oenococcus, Sporolactobacillus, Streptococcus, Staphylococcus, Saccharomyces, Pediococcus, Peptostreptococcus, Proprionebacterium, and Weissella. A wide variety of strains of bacteria are available from the ATCC (American Type Culture Collection), Manassas, Va. In an aspect, the one or more probiotic agents include at least one of a non-pathogenic strain of a pathogenic bacterium.

In an aspect, the one or more probiotic agents can include a bacterial strain that inhibits a second bacterial strain, e.g., by out competing for resources or by inhibiting the growth of the second bacterial stain. In an aspect, the one or more probiotic agents include Staphylococcus epidermidis, a skin commensal microorganism. For example, Staphylococcus epidermidis may be used as a probiotic to modulate growth of pathogenic bacteria on the skin surface by producing microbial peptides that inhibit Staphylococcus aureus biofilm formation and/or by producing lanthionine-containing antibacterial peptides, e.g., bacteriocins, which are known to exhibit antibacterial properties toward certain species of harmful bacteria, e.g., Streptococcus aureus and Streptococcus pyogenes. For example, Staphylococcus epidermidis may be used as a probiotic to stimulate the immune system by influencing the innate immune response of keratinocytes through Toll-like receptor (“TLR”) signaling. For example, Staphylococcus epidermidis may be used as a probiotic to inhibit the action of more virulent microorganisms such as Staphylococcus aureus by occupying receptors on a host cell that also bind the virulent microorganism. See, e.g., Orrice & Segre (2011) Nat. Rev. Microbiol. 9:244-53, which is incorporated herein by reference.

In an aspect, the one or more probiotic agents include Propionibacterium acnes, another skin commensal microorganism. As an example, Propionibacterium acnes can be used as a probiotic to consume skin oil and to produce byproducts such as short-chain fatty acids and propionic acid known to help maintain a healthy skin barrier. See, e.g., Shu et al. (2013) PLoS ONE 8(2):e55380, which is incorporated herein by reference.

In an aspect, the plurality of treatment agents 110 includes one or more prebiotic agents 305. In an aspect, the one or more prebiotics agents are substances that promote the survival and/or growth of microorganisms of interest on a body surface, e.g., a skin, gastrointestinal, mucosal, vaginal, nasal, or oral surface. In an aspect, the one or more prebiotic agents are substances that promote the survival and/or growth of microorganisms of interest on the skin surface of the user. In an aspect, the one or more prebiotic agents include at least one of oligosaccharides, e.g., galacto-oligosaccharides and/or fructo-oligosaccharides, inulin, or lactulose. In an aspect, the one or more prebiotic agents include at least one nutritional source, non-limiting examples of which include of iron, biotin, nicotinic acid, D-pantothenic acid, pyridoxal, pyridoxamine dihydrochloride, thiamin hydrochloride, valine, arginine, galactose, mannose, fructose, sucrose, lactose, or maltose. In an aspect, the one or more prebiotic agents include one or more plant-derived prebiotics, e.g., derived from acacia gum, konjac, chicory root, Jerusalem artichoke, asparagus, and/or dandelion greens. See, e.g., U.S. Patent Application 2013/0115317; and Bateni et al. (2013) Am. J. Dermatology Venereology 2:10-14, which are incorporated herein by reference.

In an aspect, the plurality of treatment agents 110 includes one or more antimicrobial agents 310. In an aspect, the one or more antimicrobial agents include at least one of an antibacterial agent, an antifungal agent, or an antiviral agent. In an aspect, the one or more treatment agents include one or more antibacterial agents configured to prevent or minimize a bacterial infection on a body surface of a user. In an aspect, the one or more treatment agents include one or more antibacterial agents configured to prevent or minimize a bacterial infection on a skin surface of a user. Non-limiting examples of antibacterial agents commonly used for topical applications include benzoyl peroxide, sodium sulfacetamide, erythromycin, mupirocin, retapamulin, bacitracin, neomycin, polymyxin b/e, silver sulfadiazine, or tetracycline. In an aspect, the plurality of treatment agents includes one or more antiviral agents configured to prevent or treat a viral infection. For example, at least one of the plurality of treatment agents can include an antiviral agent to prevent or treat viral infection of the skin surface associated with herpes simplex types 1 or 2. Non-limiting examples of antiviral agents commonly used for topical applications include acyclovir, docosanol, famciclovir, imiquimod, penciclovir, valacyclovir, and vidarabine. In an aspect, the plurality of treatment agents includes one or more antifungal agents configured to prevent or treat a fungal infection of a body surface, e.g., a fungal infection on the skin surface of a user. Non-limiting examples of antifungal agents commonly used for topical applications include clotrimazole, amphotericin B, butaconazole, butenafine, ciclopirox olamine, econazole, ketoconazole, miconazole, naftifine, natamycin, nystatin, oxiconazole, sulconazole, terbinafine, terconazole, tioconazole, and tolnaftate.

In an aspect, the plurality of treatment agents 110 includes one or more therapeutic agents 315. In an aspect, the one or more therapeutic agents include at least one of an anti-inflammatory agent, a chemotherapeutic agent, an antiseptic agent, an anesthetic agent, or an anti-acne agent. In an aspect, the plurality of treatment agents 110 includes one or more treatment agents to treat other conditions of a body surface, e.g., inflammation or cancer. In an aspect, the one or more treatment agents include one or more of vitamins (e.g., Vitamin A or Vitamin D), or derivatives thereof, benzoyl peroxide, salicylic acid or other acids, hormone or retinoid creams, steroids, cortisone, emollients, moisturizers, chemotherapies, e.g., 5-fluorourasil. In an aspect, the one or more treatment agents include one or more retinoids for treating various conditions of the skin including, but not limited to, acne, psoriasis, photodamaged skin and cancers including AIDS-related Kaposi's sarcoma and cutaneous T-cell lymphoma. Non-limiting examples of retinoids for topical use include alitretinoin, bexarotene, adapalene, tazarotene, and isotretinoin.

In an aspect, the plurality of treatment agents 110 includes one or more corticosteroid for treating various inflammatory dermatoses including, but not limited to, atopic dermatitis, psoriasis, lupus erythematosus, and the like. Non-limiting examples of corticosteroids for topical use include hydrocortisone and derivatives, betamethasone and derivatives, dexamethasone, prednisolone and derivatives, fluocinolone acetonide, fluorometholone, alclometasone dipropionate, triamcinolone acetonide, clocortolone pivalate, flumethasone pivalate, mometasone furoate, flurandrenolide, prednicarbate, fluticasone propionate, desonide, halcinonide, desoximetasone, flurandrenolide, fluocinonide, amcinonide, fluocinolone acetonide, and diflorasone diacetate.

In an aspect, the plurality of treatment agents 110 includes one or more chemotherapy agents for treating cancer or other conditions of the skin surface. Non-limiting examples of chemotherapy agents for topical use include fluorouracil used for treating actinic keratoses and some types of basal cell carcinomas of the skin. In an embodiment, the at least one chemotherapy agent includes an immunomodulator, non-limiting examples of which include imiquimod, tacrolimus and pimecrolimus. In an embodiment, the at least one chemotherapy includes at least one agent for modulating pigmentation, non-limiting examples of which include hydroquinone, monobenzene, mequinol, trioxsalen and methoxsalen.

In an aspect, the plurality of treatment agents 110 includes one or more antiseptic agents, non-limiting examples of which includes alcohols, e.g., ethanol or isopronanol, quaternary ammonium compounds, e.g., benzalkonium chloride, boric acid, chlorhexidine gluconate, hydrogen peroxide, super-oxidized water, iodine, povidone-iodine, octenidine dihydrochloride, phenol, and polyhexanide,

In an aspect, the plurality of treatment agents 110 includes one or more anesthetic agents. In an aspect, the one or more anesthetic agents include one or more topical anesthetic agents. Non-limiting examples of anesthetic agents include benzocaine, butamben, dibucaine, lidocaine, oxybuprocaine, pramoxine, proparacaine, proxymetacaine, or tetracaine.

In an aspect, the plurality of treatment agents 110 includes one or more buffers and/or media components for modulating and/or inhibiting microbial growth. For example, the plurality of treatment agents can include buffers that modulate the pH conditions of a body surface, e.g., the skin surface. For example, the plurality of treatment agents can include moisturizers, astringents, or drying agents that modulate the hydration conditions of a body surface, e.g., the skin surface. In an aspect, the plurality of treatment agents 110 includes at least one of a moisturizer, an astringent, an anti-aging treatment agent, a retinoid agent, or a cosmetic agent 320. In an aspect, the plurality of treatment agents includes at least one of witch hazel, calamine, rubbing alcohol, zinc oxide. In an aspect, the plurality of treatment agents includes one or more of keratoregulators, keratolytics, healing and/or restructuring agents of the cutaneous barrier, PPAR, RXR or LXR agonists, sebum-regulating agents, anti-irritation and/or anti-inflammatory and/or soothing agents, antioxidant agents, anti-aging agents, depigmenting or hypopigmenting agents, pigmenting agents, lipolytic or lipogenesis inhibitor agents or anti-cellulitis or slimming agents, organic or mineral sunscreens and filters, preservatives, and immunomodulators.

In an aspect, one or more treatment agents of the plurality of treatment agents are formulated for administration to a user. In an aspect, one or more treatment agents of the plurality of treatment agents are formulated for at least one of topical administration, oral administration, rectal administration, inhalation administration, intravaginal administration, and/or intranasal administration. In an aspect, the one or more treatment agents are formulated for topical administration as at least one of a liquid, a gel, a cream, a semi-solid, or a spray. In an aspect, the one or more treatment agents are formulated in liquid form. In an aspect, the one or more treatment agents are formulated in a liquid, emulsion, or semisolid concentrate form compatible with an aerosolized spray. For example, the one or more treatment agents can be formulated with a liquefied gas or compressed gas propellant, e.g., fluorocarbons, hydrofluorocarbons, hydrochlorofluorcarbons, carbon dioxide, nitrous oxide, or nitrogen. For example, the one or more treatment agents can be formulated as a metered dose formulation for inhalation including the one or more treatment agents, solvents, dispersing agents, and liquefied gas or compressed gas propellants. In an aspect, the one or more treatment agents are formulated in cream form. In an aspect, the one or more treatment agents are formulated in gel form. In an aspect, the one or more treatment agents are formulated in a suppository form. In an aspect, the one or more treatment agents are formulated in powder form. In an aspect, the one or more treatment agents are formulated in a solid or soft solid form which when rubbed directly on a skin surface leaves a film including the one or more treatment agents. For further reference regarding formulation of treatment agents, see Remington: The Science and Practice of Pharmacy. 21^{st} Edition (2006), Lippincott Williams & Wilkins, Philadelphia, which is incorporated herein by reference.

### Microbe Sampling Unit

The kit described herein includes a microbe sampling unit. In an aspect, the microbe sampling unit substantially conforms to a topography of the body surface of the user. For example, the microbe sampling unit can substantially conform to a topography of a skin surface of the user. In an aspect, the microbe sampling unit is personalized for the user. For example, the microbe sampling unit can be formed from a digital model of a body surface, e.g., a skin or oral surface, of the user through a three-dimensional printing process. For example, one or more images of the user, e.g., photographs, may be used by a supplier to generate a personalized microbe sampling unit, e.g., a pre-formed mask, which is either provided in the kit or as a separate item.

In an aspect, at least one surface of the microbe sampling unit includes a microbe-capture region configured to capture one or more types of microbes from a body surface of a user. The one or more types of microbes can include one or more types of bacteria, fungus, virus, parasite, archaea, or small arthropod (e.g., mites). In an aspect, the one or more types of microbes include at least one type of mutualistic microbe, commensal microbe, or pathogenic microbe. In an aspect, the one or more types of microbes include at least one type of introduced microbe, e.g., a probiotic or genetically engineered microbe. In an aspect, the one or more types of microbes include at least one type of resident microbe, e.g., at least one type of skin-resident or gut-resident microbe.

In an aspect, the microbe-capture region of the microbe sampling unit is configured to capture one or more types of microbes from a skin surface of the user. Non-limiting examples of skin-associated or skin-resident bacteria include proteobacteria, e.g., Pseudomonas sp., Janthinobacterium sp, Alphaproteobacteria, other gammaproteobacteria, and betaproteobacteria; Actinobacteria, e.g., Kocuria sp., Propionibacteria sp.; Firmicutes, e.g., Staphylococcus epidermidis; Bacteroidetes; and Spirochaetes. See, e.g., Grice et al. (2008) Genome Res. 18:1043-1050; Grice & Segre (2011) Nat. Rev. Microbiol. 9:244-253, which are incorporated herein by reference. Non-limiting examples of fungi, including skin-resident or associated types of fungi, include dermatophtyes, e.g., trichophyton, microsporum, epidermophyton, tinea capitis. Other skin-associated fungi include but are not limited to yeast, Candida, e.g., Candida albicans; and Malassezia spp (e.g., M. dermatis, M. furfur, M. globosa, and M. restricta). See, e.g., Gaitanis et al. (2012) Clin. Microbiol. Rev. 25:106-141, which is incorporated herein by reference. Non-limiting examples of skin-associated or skin-resident viruses include herpes simplex virus type I (HSV-1), herpes zoster, Molluscum contagiosum, human papillomavirus (HPV), Coxsackie virus A16, and herpes gladiatorum. Non-limiting examples of other parasites resident or associated with a skin surface include skin-associated parasitic arthropods including parasitic mites, e.g., Demodex spp including D. folliculorum and D. brevis, and Sarcoptes scabiei, a skin parasite associated with scabies.

In an aspect, the microbe-capture region of the microbe sampling unit is configured to capture one or more types of microbes from a gastrointestinal surface of the user. Non-limiting examples of gastrointestinal microbes include members of the bacterial phyla Firmicutes, Bacteroidetes, Actinobacteria, Proteobacteria, Verrucomicrobia, and Fusobacteria. See, e.g., Guinane & Cotter (2013) Ther Adv Gastroenterol 6:295-308, which is incorporated herein by reference.

In an aspect, the microbe-capture region of the microbe sampling unit is configured to capture one or more types of microbes from a mucosal surface, a vaginal surface, a nasal surface, or a mouth surface of the user. Non-limiting examples of microbes associated with a vaginal surface include Lactobacillus species such as L. crispatus, L. iners, L. jensenii, and L. gasseri; other bacteria such as Gardnerella vaginalis, Mycoplasma hominis, Atopobium vaginae, Prevotella spp., Veillonella spp., Mobiluncus spp.), uropathogens (Escherichia coli, Proteus spp., Klebsiella spp., Serratia spp.); sexually transmitted viruses (HIV, Herpes virus); and yeast species Candida albicans, Candida tropicalis, and Candida krusei. See, e.g., Mastromarino et al. (2013) New Microbiologica 36:229-238, which is incorporated herein by reference. Non-limiting examples of microbes associated with a nasal surface include members of the bacterial phyla Actinobacteria (e.g., Propionibacterium spp. and Corynebacterium spp.), Firmicutes (e.g., Staphylococcus spp.) and Proteobacteria (e.g., Enterobacter spp.). See, e.g., Frank et al. (2010) PLoS One 5(5):e10598, which is incorporated herein by reference. Non-limiting examples of microbes associated with a mouth surface include Streptococcus, Actinomyces, Veillonella, Fusobacterium, Porphromonas, Prevotella, Treponema, Nisseria, Haemophilis, Eubacteria, Lactobacterium, Capnocytophaga, Eikenella, Leptotrichia, Peptostreptococcus, Staphylococcus, Corynebacterium, Rothia, Selenomonas, Treponema, Propionibacterium, and TM7 genera 1 and 5. See, e.g., Dewhirst et al. (2010) J. Bacteriology 192:5002-5017, which is incorporated herein by reference. Non-limiting examples of fungi of the oral microbiota include Candida albicans, Aspergillus, Blastomyces dermatitidis, Cryptococcus neoformans, and Histoplasma capsulatum. Non-limiting examples of viruses of the oral microbiota include herpes simplex virus (HSV-1), human papillomavirus, coxsackievirus, and Paramyxoviridae viruses.

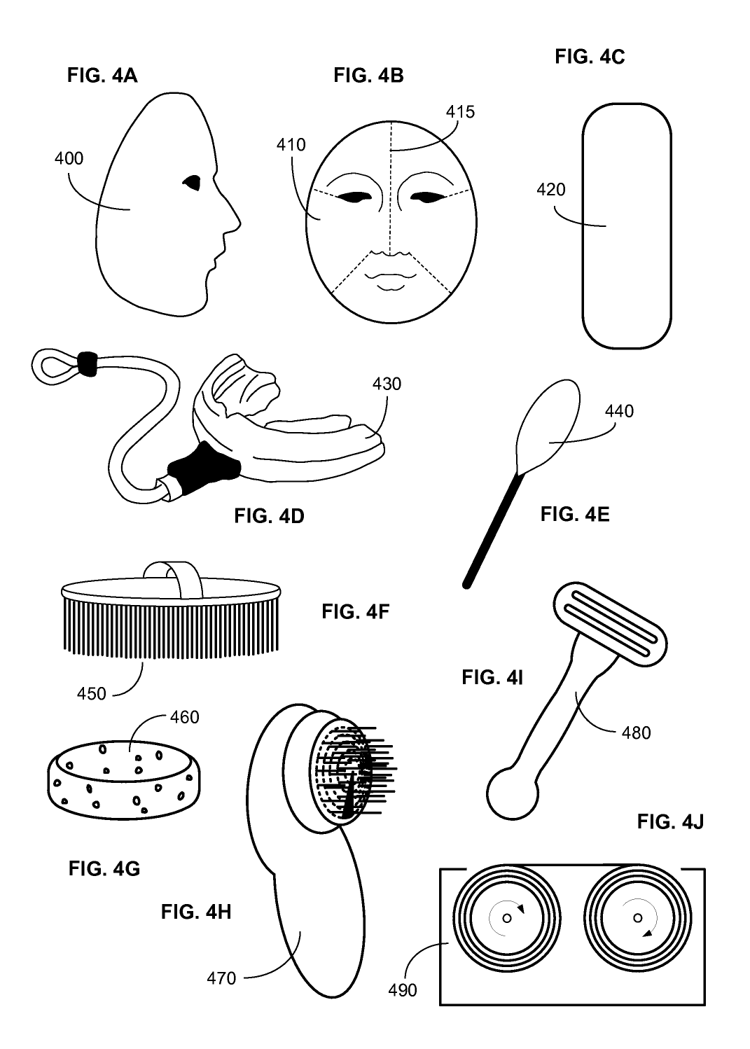

Referring back to FIG. 3 and FIGS. 4A-4J, microbe sampling unit 120 can take a number of forms. In an aspect, microbe sampling unit 120 includes mask 325. A non-limiting example mask 400 is shown in FIG. 4A. In an aspect, mask 325 substantially conforms in shape to a topography of a skin surface of a user. In an aspect, mask 325 is personalized for a given user. In an aspect, mask 325 includes pre-formed mask 330. In an aspect, pre-formed mask 330 includes a semi-rigid material, e.g., latex. In an aspect, pre-formed mask 330 includes a rigid material, e.g., a hard plastic. In an aspect, pre-formed mask 330 is formed from any of a number of materials capable of being shaped, molded, or printed, non-limiting examples of which include acrylic, nylon, plastic, ceramic, resin, rubber, epoxy, thermoplastic, polymer, photopolymer, gel, hydrogel, latex, or silicone. In an aspect, the microbe sampling unit includes a mask 325 that is a peelable mask 335. In an aspect, peelable mask 335 includes a flexible solid. In an aspect, peelable mask 335 includes a settable material, e.g., at least one of latex, gel, polymer, plastic, resin, that is applied to a body surface, e.g., a skin surface, and allowed to set. In an aspect, the settable material is configured to undergo a phase change from a liquid or gelled phase to a flexible solid phase in response to an applied stimulus, e.g., exposure to air, thermal stimulus, or an electromagnetic stimulus. In an aspect, mask 325, pre-formed mask 330, and/or peelable mask 335 can include a mask 410 of FIG. 4B with one or more tearable lines 415, e.g., perforations, to allow a three-dimensional mask to be flattened for analysis. Other non-limiting aspects of a mask for use as a microbe sampling unit are described in U.S. patent application Ser. No. 13/975,055, which is incorporated herein by reference.

In an aspect, microbe sampling unit 120 includes mouthpiece 340. A non-limiting example mouthpiece 430 is shown in FIG. 4D. In an aspect, mouthpiece 340 includes one or more surfaces that substantially conform in shape to a topography of at least a portion of the interior mouth region of a user. In an aspect, mouthpiece 340 includes a pre-formed or a peelable mouthpiece formed from any of a number of materials capable of being shaped, molded, or printed, non-limiting examples of which have been described above herein. In an aspect, mouthpiece 340 is formed from a settable material, e.g., sodium alginate, polyether, silicones, polyvinyl siloxane, agar, or zinc oxide eugenol, using a dental or impression tray. In an aspect, at least one surface of mouthpiece 340 includes a microbe-capture region. For example, that portion of the mouthpiece that comes into contact with the walls of the mouth can include a microbe-capture region configured to capture one or more types of microbes. Other non-limiting aspects of a mouthpiece for use as a microbe sampling unit are described in U.S. patent application Ser. No. 13/975,055, which is incorporated herein by reference.

In an aspect, microbe sampling unit 120 includes strip 345. A non-limiting example strip 420 is shown in FIG. 4C. In an aspect, strip 345 includes a thin piece of material coated on at least one surface with a microbe-capture region. For example, the strip can include a specially coated strip of bendable material, e.g., a coated sheet of flexible plastic, polymer, nitrocellulose, fabric, or paper. In an aspect, strip 345 can be configured to substantially conform in shape to the topography of a body surface, e.g., a skin surface. For example, the strip can wrap around the contours of a body region, e.g., around the contours of a face, an extremity, or lumen of the gastrointestinal tract, vagina, mouth, or nasal cavity. In an aspect, the strip includes a flexible strip similar to a wound covering but with a microbe-capture region configured to capture one or more types of microbes.

In an aspect, microbe sampling unit 120 includes swab 350. A non-limiting example swab 440 is shown in FIG. 4E. In an aspect, swab 350 includes a small wad of absorbent material, e.g., cotton, on the end of a short rod. In an aspect, swab 350 includes a small piece of gauze, cotton ball, or absorbent wipe. In an aspect, at least one surface of the gauze, cotton ball, or absorbent wipe includes a microbe-capture region, either as an inherent property of the material of the swab or as a material added to the swab. In an aspect, microbe sampling unit 120 includes sponge 460 shown in FIG. 4G. In an aspect, at least one surface of sponge 460 includes a microbe-capture region. In an aspect, sponge 460 is attached to a short rod.

In an aspect, microbe sampling unit 120 includes a brush 355. A non-limiting example brush 450 is shown in FIG. 4F. In an aspect, brush 355 includes bristles, wire, or other filaments attached to a handle, the bristles, wire, or other filaments configured to capture one or more types of microbes. In an aspect, the bristles are natural, e.g., natural hair, sable, or hog, or synthetic, e.g., polyester, nylon, or a combination thereof. In an aspect, the bristles, wire, or other filaments of the brush are coated with a material to form a microbe-capture region.

In an aspect, microbe sampling unit 120 includes a razor 360. A non-limiting example razor 480 is shown in FIG. 4I. In an aspect, razor 360 includes one or more blades for scraping one or more types of microbes from a body surface, e.g., a skin surface. In an aspect, at least one surface of the one or more blades includes a microbe-capture region.

In an aspect, the microbe sampling unit and the analyzer are incorporated into a hand-held microbe profiling device 365. In an aspect, a hand-held microbe profiling device includes a device head including a body surface-engaging component, e.g., an epidermis-engaging component, and at least one access window, the device head configured to dislodge at least one type of microbe from a body surface of an individual. In an aspect, the device head is attached to a hand-held housing. In an aspect, at least a portion of the hand-held housing defines an opening aligned with the at least one access window of the device head, the hand-held housing including a microbe sampling unit, e.g., a substrate, with a microbe-capture region. The hand-held housing further includes at least one sensor component including circuitry configured to detect one or more signals emitted or reflected from the microbe-capture region of the microbe sampling unit and to transform the detected one or more signals into a sensor output, a user interface, and a computing component operably coupled to the at least one sensor component and the user interface. The computing component of the hand-held profiling device includes circuitry configured to receive the sensor output from the at least one sensor component, the sensor output including information associated with at least one property of the detected one or more signals emitted or reflected from the microbe-capture region of the microbe sampling unit; circuitry configured to compare the at least one property of the detected one or more signals emitted or reflected from the microbe-capture region of the microbe sampling unit with a reference dataset of signal properties; circuitry configured to generate a microbe profile of the user based on the comparison with the reference dataset of signal properties; circuitry configured to compare the microbe profile of the user with at least one reference microbe profile; and circuitry configured to recommend to the user at least one of the plurality of treatment agents in the kit based one the comparison with the at least one reference microbe profile. In some embodiments, the hand-held housing further includes a motor operably coupled to at least one motivatable component, the motor including circuitry configured to drive the at least one motivatable component; at least a portion of the microbe sampling unit disposed in relation to the at least one motivatable component and configured to be in operable communication with the opening defined by the hand-held housing; and a location capture component including circuitry configured to determine a location of one or more regions of a body surface, e.g., a skin surface, of the user as the epidermis-engaging component of the device contacts said one or more regions of the body surface. In an aspect, the computing component further includes circuitry configured to receive information associated with a location of one or more regions of the body surface of the user from the location-capture component; receive the sensor output from the at least one sensor component; associate the location of said one or more regions of the body surface of the user with the detected one or more signals; and output information regarding an association between the location of said one or more regions of the body surface of the user and the detected one or more signals. In an aspect, the hand-held profiling device includes a vacuum, the vacuum configured to pull one or more types of microbes from a body surface and into the hand-held microbe profiling device for analysis. Other non-limiting aspects of hand-held microbe profiling devices and systems are described in U.S. patent application Ser. No. 14/091,762, which is incorporated herein by reference.

In an aspect, hand-held microbe profiling device 365 includes brush head 370. A non-limiting example of a hand-held microbe profiling device with a brush head 470 is shown in FIG. 4H. In an aspect, hand-held microbe profiling device 355 includes razor head 375. In an aspect, hand-held microbe profiling device 365 includes rotatable microbe sampling unit 380. For example, rotatable microbe sampling unit 380 can include a rotating substrate, at least one surface of the rotating substrate include a microbe-capture region. In an aspect, rotatable microbe sampling unit 380 is a cassette with one or more rotatable components. A non-limiting example of a cassette 490 with a rotatable microbe-capture region is shown in FIG. 4J. Other non-limiting aspects of a hand-held microbe profiling device with a rotatable microbe sampling unit are described in U.S. patent application Ser. No. 14/091,832, which is incorporated herein by reference. In an aspect, a hand-held microbe profiling device includes an analyzer and a replaceable microbe sampling unit, e.g., a replaceable substrate including a microbe-capture region.

In an aspect, microbe sampling unit 120 includes at least one optical sensor 385. For example, the microbe sampling unit can include a mask with at least one optical sensor embedded in a surface of the mask. In an aspect, the at least one optical sensor of the microbe sampling unit includes at least one photodetector. In an aspect, the at least one optical sensor of the microbe sampling unit includes at least one charged-coupled device, photodiode, quantum dot photoconductors or photodiodes, complementary metal-oxide-semiconductor (CMOS) device, active-pixel sensors, reverse-biased light emitting diode, or any other sensor type capable of detecting an optical electromagnetic signal. In an aspect, the microbe sampling unit includes an electromagnetic energy emitter, e.g., at least one light emitting diode, as well as at least one optical sensor configured to detect one or more signals emitted or reflected from a body surface of the individual. In an aspect, an electromagnetic energy emitter of the microbe sampling unit elicits a light-emitting response from the body surface of the individual. In an aspect, the electromagnetic energy emitter of the microbe sampling unit elicits a fluorescence response from at least one signal-generating element, e.g., a fluorescence signal-generating agent, associated with microbes on the body surface. For example, the body surface of the individual can be treated with a fluorescence signal-generating element, e.g., a fluorescing antibody, that interacts with one or more types of microbes, the interaction detected with the at least one optical sensor of the microbe sampling unit. For example, the microbe sampling unit can include a light source of appropriate wavelength to elicit a fluorescence response from one or more types of fluorescently labeled microbes. In an aspect, an electromagnetic energy emitter of the microbe sampling unit elicits an autofluorescence response from one or more types of microbes on the body surface of the individual. For example, the microbe sampling unit can include a light source of appropriate wavelength to elicit an autofluorescence response from one or more types of microbes on the skin surface of a user. See, e.g., U.S. Pat. Nos. 8,109,875 and 8,280,471 and U.S. Patent Application Nos. 2011/0117025 and 2010/0185064, which are incorporated herein by reference. In an aspect, the response elicited by the electromagnetic energy emitter, e.g., one or more signals emitted or reflected the on the body surface, is detected by the at least one optical sensor. In an aspect, the microbe sampling unit including the at least one optical sensor is operably coupled to the analyzer of the kit, e.g., through a wireless communication link, the analyzer including circuitry configured to receive sensor output from the at least one optical sensor of the microbe sampling unit, the sensor output including at least one property of the one or more signals emitted or reflected from the body surface of the individual, compare the at least one property of the detected one or more signals emitted or reflected from the body surface of the individual with a reference dataset of signal properties, generate a microbe profile of the user based on the comparison with the reference dataset of signal properties, compare the microbe profile of the user with at least one reference microbe profile, and recommend to the user at least one of the plurality of treatment agents based on the comparison with the at least one reference microbe profile.

In an aspect, the microbe sampling unit including the microbe-capture region is a consumable, intended for single use. For example, a microbe sampling unit that is a mask, pre-formed mask, peelable mask, mouthpiece, strip, swap, brush, or razor may be designed for single use. In an aspect, the microbe sampling unit is intended for multiple uses with a replaceable/renewable microbe-capture region. For example, the kit can include one or more components of a microbe-capture region that are sprayed or coated onto at least one surface of a washable microbe sampling unit prior to each use.

### Microbe-Capture Region

The microbe sampling unit has at least one surface including a microbe-capture region. In an aspect, the entirety of the microbe sampling unit includes a microbe-capture region. For example, all external surfaces of the microbe sampling unit may include a microbe-capture region capable of capturing one or more types of microbes from a body region, e.g., a skin surface, of an individual. In an aspect, only one surface of the microbe sampling unit includes a microbe-capture region. For example, the microbe sampling unit may include one surface intended to come in contact with a body region, e.g., a skin surface of an individual, which includes the microbe-capture region. In an aspect, the microbe sampling unit includes more than one microbe-capture region on a given surface. For example, multiple microbe-capture regions may be patterned, e.g., striped, on a surface of the microbe sampling unit. In an aspect, the microbe sampling unit may have many surfaces, e.g., the many bristle surfaces of a brush or the fibrous texture of a cotton swab, at least a portion of the many surfaces including a microbe-capture region. In an aspect, the microbe-capture region is configured to non-selectively capture one or more types of microbes. For example, the microbe-capture region may include a material, e.g., an adhesive, which non-selectively binds all microbes from a body region. In an aspect, the microbe-capture region is configured to specifically capture one or more types of microbes. For example, the microbe-capture region may include a plurality of specific microbe-binding elements that specifically capture one or more types of microbes. In an aspect, the microbe-capture region is configured to generate a signal in response to interaction, e.g., binding, with one or more types of microbes. For example, the microbe-capture region can include a plurality of signal-generating complexes that emit a signal in response to interaction with one or more types of microbes.

In an aspect, the microbe-capture region is replaceable. For example, the microbe sampling unit may be intended for multiple uses with a replaceable microbe-capture region. In an aspect, the microbe-capture region includes at least one consumable. In an aspect, the kit described herein includes a consumable microbe-capture region. For example, the kit can include at least one consumable that is a consumable liquid, spread, or spray containing one or more materials for applying a microbe-capture region to a surface of the microbe sampling unit. For example, the microbe sampling unit may include a washable surface which is repeatedly coated with one or more materials to form the microbe-capture region. For example, the microbe-capture region may include a material, e.g., an adhesive, which can be removed, e.g., washed off, from a surface of the microbe sampling unit after a first use and replaced with a fresh coating of adhesive for one or more subsequent uses. For example, the microbe-capture region may include one or more strips of material that can be removed from a surface of the microbe sampling unit and replaced with a new strip of material. For example, the microbe-capture region may include multiple strips of material, and after each use, the used strip is removed revealing a fresh, underlying strip.

In an aspect, the microbe-capture region covers the entire outer surface of the microbe sampling unit. In an aspect, the microbe-capture region covers at least a portion of the outer surface of the microbe sampling unit. In an aspect, the microbe-capture region is an integral part of the microbe sampling unit, e.g., the entirety of the microbe sampling unit has microbe-capturing properties. For example, one or more materials used to form the microbe sampling unit may include properties, e.g., “tackiness” or charge properties, which allow for non-selective capture of one or more types of microbes from a skin surface. For example, the microbe sampling unit may include a gel-like material that non-selectively captures one or more types of microbes.

In an aspect, the microbe-capture region forms a separate layer on a surface of the microbe sampling unit. For example, the microbe-capture region may include a material, e.g., a liquid, a gel, a coating, or a spray which is spread on a surface of the microbe sampling unit to generate the microbe-capture region. For example, the microbe-capture region may include an adhesive material that is added to a surface of the microbe sampling unit. For example, the microbe-capture region may include a plurality of specific microbe-binding elements that is layered onto a plastic film.

### Microbe-Capture Region—Non-Selective

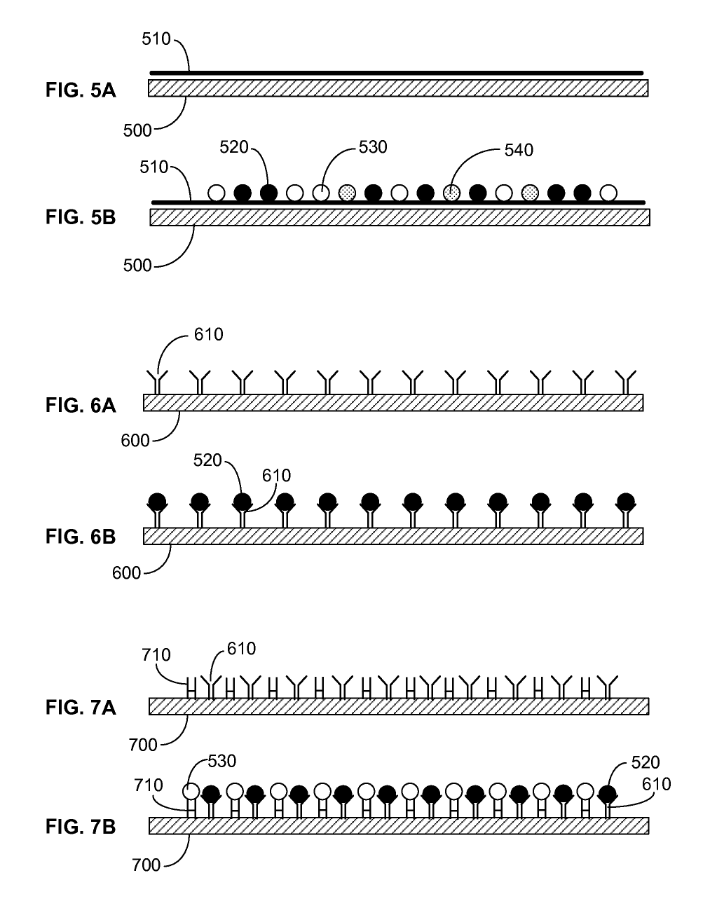

In an aspect, the microbe-capture region includes a non-selective microbe capture region. With reference to FIGS. 5A and 5B, a microbe sampling unit can include at least one surface 500 that includes non-selective microbe-capture region 510. In an aspect, microbe-capture region 510 includes one or more materials configured to non-selectively capture microbes from a body surface of a user. For example, FIG. 5B shows surface 500 of a microbe sampling unit with first type of microbe 520, second type of microbe 530, and third type of microbe 540 captured on non-selective microbe-capture region 510. In an aspect, the non-selective microbe-capture region includes one or more materials configured to non-selectively capture microbes from the skin surface of a user. In an aspect, the microbe-capture region includes one or more materials configured to non-selectively capture a representative sample of the microbes, i.e., a representative sample of all microbes, on the skin surface of the individual. In an aspect, the microbe-capture region includes one or more materials configured to non-selectively capture a subtype of microbe, e.g., all of a type of microbe, for example, bacteria versus fungi. In general, the microbe-capture region can include one or more materials that interact with biomolecules on the outer surface of microbes, e.g., proteins, polysaccharides, carbohydrates, phospholipids, proteoglycans, and the like. In an aspect, the one or more materials take advantage of hydrogen bonding, electrostatic and/or hydrophobic interactions to capture microbes onto the microbe-capture region. Non-limiting examples of materials for use in a microbe-capture region include poly-ionic surfaces, e.g., poly-cationic surfaces such as polyamino acids (e.g., polylysine) and fibronectin for binding microbes that have an overall negative surface charge. Other non-limiting examples of materials for use in a microbe-capture region include nitrocellulose, cellulose nitrate, hydrophobic polymers, polyvinylidene fluoride coating, nylon coating, streptavidin or bioting, proteins, peptides, Concanavalin A, epoxy for binding proteins and peptides, aldehydes for immobilizing amino modified oligos and cDNAs, native proteins, tissues, and cells, and amines for immobilizing long oligos and cDNAs.

In an aspect, the microbe-capture region of the microbe sampling unit includes a charged surface, e.g., a positively charged surface. In an aspect, the positive charge is provided by the one or more materials used to form the microbe sampling unit. In an aspect, the positive charge is provided by a positively charged material used to coat at least the outer surface of the microbe sampling unit to form the microbe-capture region. For example, polymers of secondary and tertiary amino groups can be used to create a positively charged surface capable of binding bacteria. See, e.g., Terada et al. (2006) Microbiology 152:3575-3583, which is incorporated herein by reference. For example, poly-L-lysine, a highly positively charged amino acid chain, can be used to bind microbes to a surface. See, e.g., Cowan et al. (2001) Biotechnology Letters 23:1235-1241, which is incorporated herein by reference. For example, the positively charged surface can include cationic polymer, e.g., Kymene® or a responsive polymer. See, e.g., U.S. Patent Application 2007/0134337 and WO2010094976, which are incorporated herein by reference.

In an aspect, the microbe-capture region of the microbe sampling unit includes at least one of an adhesive, an absorbent, or an adsorbent. For example, the microbe-capture region can include an adhesive or sticky substance that non-selectively captures microbes. In an aspect, the adhesive can include one or more pressure-sensitive adhesive, e.g., adhesive tape. Non-limiting examples of adhesives designed for healthcare use include any of a number of silicone-based pressure sensitive adhesives from, for example, Dow Corning, Midland, Mich. or 3M, St. Paul, Minn. In an aspect, the adhesive forms a separate layer on at least the outer surface of the microbe sampling unit. For example, a biocompatible adhesive may be applied to the outer surface of a long and narrow piece of Mylar or comparable piece of plastic film. For example, a biocompatible adhesive may be applied to the at least one surface of a mask, bristles of a brush, or blades of a razor.

In an aspect, the microbe-capture region of the microbe sampling unit includes a biomolecule-binding polymer. In an aspect, the biomolecule-binding polymer includes a form of cellulose, e.g., nitrocellulose. Binding of biomolecules, e.g., proteins, to nitrocellulose is by a combination of weak intermolecular forces, probably dominated by hydrophobic and van der Waals interactions. In an aspect, biomolecule-binding polymer includes agarose, starch, cellulose acetate, or polyacrylamide. In an aspect, the biomolecule-binding polymer includes one or more polyamino acids. Non-limiting examples of polyamino acids include poly-L-lysine, poly-D-lysine, poly-L-ornithine. For example, poly-L-lysine contains positively charged hydrophilic amino groups that electrostatically bind to the cell surface of bacteria and other cell types.

In an aspect, the microbe-capture region of the microbe sampling unit includes one or more biological materials associated with an extracellular matrix, non-limiting examples of which include collagen, laminin, fibronectin, mucopolysaccharides, heparin sulfate, hyaluronidate, and chondroitin sulfate. In an aspect, the microbe-capture region includes albumin. See, e.g., de Chateau et al. (1996) J. Biol. Chem. 271:26609-26615, which is incorporated herein by reference.

In an aspect, the microbe-capture region of the microbe sampling unit includes one or more microbe-binding lipids. For example, one or more glycosphingolipids and/or one or more phospholipids can be attached to at least the outer surface of microbe sampling unit, e.g., a piece of polyvinylidene fluoride membrane, to form the microbe-capture region.

In an aspect, the microbe-capture region of the microbe sampling unit includes a gel. Non-limiting examples of gels include at least one of a hydrogel, a colloid, agar, or gelatin. In an aspect, the outer surface of the microbe sampling unit is coated with a gel. In an aspect, the entirety of the microbe sampling unit is comprised of a semi-rigid gel. In an aspect, the microbe-capture region includes agar as a layer on the outer surface of the microbe sampling unit. For example, the microbe sampling unit may be coated on the outer surface with a thin layer of gel, e.g., agar, to form the microbe-capture region.

### Microbe-Capture Region—Plurality of Specific Microbe-Binding Elements

In an aspect, the microbe-capture region is configured to capture a specific type or types of microbes. In an aspect, the microbe-capture region of the microbe sampling unit includes a plurality of specific microbe-binding elements. With reference to FIGS. 6A and 6B, a microbe sampling unit can include at least one surface 600 that includes a microbe-capture region including a plurality of specific microbe-binding elements 610. FIG. 6B shows at least one surface 600 of a microbe sampling unit with a microbe-capture region that includes a plurality of specific microbe-binding elements 610 specifically bound to first type of microbe 520. In an aspect, the microbe-capture region includes a plurality of specific microbe-binding elements that specifically recognize at least one type of microbe, e.g., at least one type of bacteria, fungus, or virus. In an aspect, each of the plurality of specific microbe-binding elements recognizes at least one type of mutualistic microbe, commensal microbe, or pathogenic microbe. In an aspect, each of the plurality of specific microbe-binding elements recognizes at least one type of microbe resident on the body surface of an individual. In an aspect, each of the plurality of specific microbe-binding elements recognizes at least one type of microbe resident on the skin surface of the individual. In an aspect, the specific microbe-binding element is configured to specifically recognize and bind a particular microbe or class of microbes. In an aspect, the specific microbe-binding element may be specific for a particular type of microbe, e.g., bacteria versus fungus. In an aspect, the specific microbe-binding element may be specific for Gram-positive versus Gram-negative bacteria or for a particular genus of microbes, e.g., Propionibacterium versus Staphylococcus. In an aspect, the specific microbe-binding element may be specific for a particular species of bacteria within a genus, e.g., S. aureus versus S. epidermidis.

In an aspect, the microbe-capture region includes a plurality of specific microbe-binding elements of at least one first type and a plurality of specific microbe-binding elements of at least one second type. In an aspect, the plurality of specific microbe-binding elements of at least one first type recognize at least one first type of microbe and the plurality of specific microbe-binding elements of at least one second type recognize at least one second type of microbe. With reference to FIGS. 7A and 7B, at least one surface 700 of a microbe sampling unit is shown including a plurality of specific microbe-binding elements of a first type 610 and a plurality of specific microbe-binding elements of a second type 710. FIG. 7B shows at least one surface 700 of a microbe sampling unit with a microbe-capture region that includes a plurality of specific microbe-binding elements of a first type 610 bound to first type of microbe 520 and a plurality of specific microbe-binding elements of a second type 710 bound to second type of microbe 530. In an aspect, the plurality of specific microbe-binding elements of the at least one first type differs from the plurality of specific microbe-binding elements of the at least one second type but the at least one first type of microbe does not differ from the at least one second type of microbe. For example, a specific antibody and a specific aptamer or a specific first antibody and a specific second antibody can be used to capture a single specific type of microbe, e.g., Staphylococcus. In an aspect, the plurality of specific microbe-binding elements of the at least one first type differ from the plurality of specific microbe-binding elements of the at least one second type and the at least one first type of microbe differs from the at least one second type of microbe. For example, the microbe sampling unit may include a first antibody that specifically recognizes and binds Propionibacterium and a second antibody that specifically recognizes and binds Staphylococcus. In this manner, two or more types of microbes can be specifically captured by the microbe-capture region and analyzed.

In an aspect, the plurality of specific microbe-binding elements includes a plurality of specific microbe-binding elements of a single type. In an aspect, “a single type” refers to a type of specific microbe-binding elements, e.g., an antibody versus an aptamer. In an aspect, “a single type” refers to a specific antibody, e.g., a monoclonal antibody with a specific protein sequence or an aptamer with a specific nucleotide sequence. In an aspect, the plurality of specific microbe-binding elements includes a plurality of specific microbe-binding elements of one or more types. In an aspect, the “one or more types” refers to an antibody versus an aptamer. In an aspect, the “one or more types” refers to one or more distinct antibodies with distinct protein sequences and/or recognition specificities or one or more distinct aptamers with distinct nucleotide sequences and/or recognition specificities.

Specific microbe-binding elements can include substances derived from natural or synthetic sources. Non-limiting examples of specific microbe-binding elements include antibodies, aptamers, oligonucelotides, or anti-16S rRNA ligands. Other non-limiting examples of specific microbe-binding elements include antibody fragments, peptides, DNA, RNA, peptide nucleic acids, proteins, viruses, lipid, glycolipids, sphingolipids, phospholipids, carbohydrates, enzymes, receptors, lectins, peptide aptamers, bacteria, cells, cell fragments, inorganic molecules, organic molecules, artificial binding substrates (e.g., those formed by molecular imprinting), or combinations thereof.

In an aspect, each specific microbe-binding element recognizes one or more components of at least one type of microbe. In an aspect, the specific microbe-binding element recognizes one or more biomolecules associated with the surface of a microbe, e.g., bacteria, a virus, or a fungus. In an aspect, the specific microbe-binding element recognizes components of microbe surface biomolecules including amino acid sequences, oligosaccharides, proteoglycans, proteins, peptides, and/or lipids. For example, the specific microbe-binding element can recognize and bind teichoic acids and/or peptidoglycans associated with Gram-positive bacteria. For example, the specific microbe-binding element can recognize and bind common lipopolysaccharide moieties, e.g., 2-keto-3-deoxyoctanate, associated with Gram-negative bacteria. For example, the specific microbe-binding element can recognize and bind chitin associated with fungi. In an aspect, the specific microbe-binding element recognizes nucleic acids. For example, the specific microbe-binding element may be configured to recognize and bind one or more DNA or RNA sequences associated with the at least one type of microbe.

In an aspect, the specific microbe-binding element recognizes one or more biomolecules associated with the bacterial outer membrane, cell wall, and/or cytoplasmic membrane. Non-limiting examples of biomolecules associated with the bacterial outer membrane of Gram-negative bacteria include, but are not limited to, lipopolysaccaride and OMP (outer membrane protein) porins, the latter of which are exemplified by OmpC, OmpF and PhoP of E. coli. Non-limiting examples of biomolecules associated with the bacterial cell wall of both Gram-positive and Gram-negative bacterial include, but are not limited to, peptidoglycans, i.e., polymers composed of an alternating sequence of N-acetylglucoamine and N-acetyl-muraminic acid and crosslinked by amino acids and amino acid derivatives. Non-limiting examples of biomolecules associated with the bacterial cytoplasmic membrane include, but are not limited to, the MPA1-C (also called polysaccharide copolymerase, PCP2a) family of proteins, the MPA2 family of proteins, and the ABC bacteriocin exporter accessory protein (BEA) family of proteins. Other examples of biomolecules associated with bacteria include, but are not limited to, transporters, e.g., sugar porter (major facilitator superfamily), amino-acid/polyamine/organocation (APC) superfamily, cation diffusion facilitator, resistance-nodulation-division type transporter, SecDF, calcium:cation antiporter, inorganic phosphate transporter, monovalent cation:proton antiporter-1, monovalent cation:proton antiporter-2, potassium transporter, nucleobase:cation symporter-2, formate-nitrite transporter, divalent anion:sodium symporter, ammonium transporter, and multi-antimicrobial extrusion; channels, e.g., major intrinsic protein, chloride channel, and metal ion transporter; and primary active transporters, e.g., P-type ATPase, arsenite-antimonite efflux, Type II secretory pathway (SecY), and sodium-transporting carboxylic acid decarboxylase. A number of other potential biomolecules associated with bacteria have been described in Chung, et al. (2001) J. Bacteriology 183:1012-1021, which is incorporated herein by reference.

In an aspect, the specific microbe-binding element recognizes one or more biomolecules associated with at least one type of fungus. Non-limiting examples of biomolecules associated with fungi, e.g., biomolecules associated with the outer surface of fungi, include chitins and glucans, e.g., alpha glucans (dextran, glycogen, pullulan, starch) and beta glucans (cellulose, curdlan, laminarin, chrysolaninarin, lentinan, lichenin, pleuran, zymosan).

In an aspect, the specific microbe-binding element recognizes one or more biomolecules associated with at least one type of virus. For example, the specific microbe-binding element may be configured to recognize one or more capsid proteins of a virus. For example, the specific microbe-binding element may be configured to recognize VP5, a major capsid protein of herpes viruses.

In an aspect, at least one of the plurality of specific microbe-binding elements includes a specific microbe-binding antibody. For example, the specific microbe-binding element can include a specific microbe-binding antibody able to recognize and bind one or more bacterium, fungus, and/or virus. Antibodies or fragments thereof for use in generating the specific microbe-binding element can include, but are not limited to, monoclonal antibodies, polyclonal antibodies, Fab fragments of monoclonal antibodies, Fab fragments of polyclonal antibodies, F(ab′)₂ fragments of monoclonal antibodies, F(ab′)₂ fragments of polyclonal antibodies, chimeric antibodies, non-human antibodies, fully human antibodies, and synthetic antibodies among others. Single chain or multiple chain antigen-recognition sites can be used. Multiple chain antigen recognition sites can be fused or unfused. Antibody fragments can be produced by modification of whole antibodies or synthesized de novo using recombinant DNA technologies. Antibodies or fragments thereof may be generated using standard methods. In some embodiments, antibodies directed against specific microbes may be available from a commercial source (from e.g., Novus Biological, Littleton, Colo.; Sigma-Aldrich, St. Louis, Mo.; United States Biological, Swampscott, Mass.). Non-limiting sources of antibodies designed to bind specific microbes, e.g., specific bacteria, fungi, viruses, or parasites, can be found in Linscott's Directory of Immunological and Biological Reagents (accessible through the website address http://www.linscottsdirector.com/).

In an aspect, at least one of the plurality of specific microbe-binding elements includes a specific microbe-binding oligonucleotide. In an aspect, a specific microbe-binding oligonucleotide includes a specific microbe-binding aptamer. The specific microbe-binding aptamer can be an oligonucleotide RNA- or DNA-based aptamer configured to recognize and bind one or more of a bacteria, fungus, virus, or parasite. Aptamers may be isolated from a large library of 10¹⁴ to 10¹⁵ random oligonucleotide sequences using an iterative in vitro selection procedure termed “systemic evolution of ligands by exponential enrichment” (SELEX). See, e.g., Cao, et al (2005) Current Proteomics 2:31-40; Proske et al. (2005) Appl. Microbiol. Biotechnol. 69:367-374, which are incorporated herein by reference. In general, SELEX may be used to generate aptamers against any of a number of microbial targets, including but not limited to bacteria, fungi, viruses, and parasites. See, e.g., Chen et al. (2007) Biochem. Biophys, Res. Commun. 357:743-748, Nitsche et al. (2007) BMC Biotechnol. 7:48; Gopinath et al. (2012) J. Virol. 86:6732-6744; Low et al. (2009) Biochem. Biophys, Res. Commun. 386:544-548, which are incorporated herein by reference.

In an aspect, at least one of the plurality of specific microbe-binding elements includes a protein, a peptide, DNA, RNA, a lectin, a carbohydrate, an anti-16S rRNA ligand, an aptamer, a synthetic ligand, or a mimetic binding element.

In an aspect, at least one of the plurality of specific microbe-binding elements includes a specific microbe-binding DNA or RNA. In an aspect, the specific microbe-binding DNA or RNA includes at least one of double-stranded DNA, single-stranded DNA, DNA-RNA hybrid, RNA, messenger RNA, transfer RNA, ribosomal RNA, transfer-messenger RNA, small interfering RNA, micro RNA, single-strand RNA, or double-stranded RNA.

In an aspect, at least one of the plurality of specific microbe-binding elements includes a novel peptide configured to specifically recognize and bind one or more microbes. Novel peptides that bind specific targets, e.g., a surface component of a bacteria, virus, or fungi, can be generated, for example, using phage display methodologies. See, e.g., Spear, et al. (2001) Cancer Gene Ther. 8:506-511, which is incorporated herein by reference.

In an aspect, at least one of the plurality of specific microbe-binding elements includes a ligand that specifically recognizes one or more microbes. For example, the specific microbe-binding element can include CD14 to bind lipopolysaccharide associated with Gram-negative bacteria and/or lipoteichoic acid associated with the Gram-positive bacteria Bacillus subtilis (see, e.g., Fan, et al. (1999) Infect. Immun. 67: 2964-2968). In an aspect, the specific microbe-binding element can include all or part of a pattern recognition receptor that recognizes microbe-specific molecules (e.g., bacterial carbohydrates, bacterial or viral DNA or RNA, bacterial peptides, peptidoglycans, lipoteichoic acids, N-formylmethionine, lipoproteins, and fungal glucans). Non-limiting examples of pattern recognition receptors with microbe-binding properties include toll-like receptors, C-type lectin receptors, NOD-like receptors, RIG-I-like receptors, RNA helicases, complement receptors, collectins, ficolins, pentraxins, C-reactive proteins, lipid transferases, and the like. See, e.g., Modlin (2012) J. Invest. Dermatol. 132:882-886; Gauglitz et al. (2012) Acta Derm. Venereol. 92:291-298, which are incorporated herein by reference.

In an aspect, at least one of the plurality of specific microbe-binding elements includes plasminogen, which recognizes and binds to a fungus, e.g., Candida albicans. See, e.g., Crowe et al. (2003) Mol. Microbiol. 47:1637-1651, which is incorporated herein by reference.

In an aspect, at least one of the plurality of specific microbe-binding elements includes a lectin. Lectins include carbohydrate-binding proteins that bind cell surface glycoproteins and/or glycolipids. Non-limiting examples of lectins include algal lectins, e.g., b-prism lectin; animal lectins, e.g., tachylectin-2, C-type lectins, C-type lectin-like proteins, calnexin-calreticulin, capsid protein, chitin-binding protein, ficolins, fucolectin, H-type lectins, I-type lectins, sialoadhesin, siglec-5, siglec-7, micronemal protein, P-type lectins, pentrxin, b-trefoil, galectins, congerins, selenocosmia huwena lectin-I, Hcgp-39, Ym1; bacterial lectins, e.g., Pseudomonas PA-IL, Burkholderia lectins, chromobacterium CV-IIL, Pseudomonas PA IIL, Ralsonia RS-ILL, ADP-ribosylating toxin, Ralstonia lectin, Clostridium hemagglutinin, botulinum toxin, tetanus toxin, cyanobacterial lectins, FimH, GafD, PapG, Staphylococcal enterotoxin B, toxin SSL11, toxin SSL5; fungal and yeast lectins, e.g., Aleuria aurantia lectin, integrin-like lectin, Agaricus lectin, Sclerotium lectin, Xerocomus lectin, Laetiporus lectin, Marasmius oreades agglutinin, agrocybe galectin, coprinus galectin-2, Ig-like lectins, L-type lectins; plant lectins, e.g., alpha-D-mannose-specific plant lectins, amaranthus antimicrobial peptide, hevein, pokeweed lectin, Urtica dioica UD, wheat germ WGA-1, WGA-2, WGA-3, artocarpin, artocarpus hirsute AHL, banana lectin, Calsepa, heltuba, jacalin, Maclura pomifera MPA, MornigaM, Parkia lectins, abrin-a, abrus agglutinin, amaranthin, castor bean ricin B, ebulin, mistletoe lectin, TKL-1, cyanovirin-N homolog, and various legume lectins; and viral lectins, e.g., capsid protein, coat protein, fiber knob, hemagglutinin, and tailspike protein. See, e.g., Kumar & Mittal (2011) Bioinformation 6:134-136, which is incorporated herein by reference.

In an aspect, the at least one of the plurality of specific microbe-binding elements includes an artificial binding substrate formed by the process of molecular imprinting. For example, an artificial binding substrate can be formed by combining a template, e.g., a microbe or part thereof, with functional monomers, e.g., acrylamide and ethylene glycol dimethacrylate, and cross-linking the monomers to form a polymer matrix that surrounds the template. Removal of the template leaves a stable cavity in the polymer matrix that is complementary in size and shape to the template. See, e.g., Alexander, et al. (2006) J. Mol. Recognit. 19:106-180, which is incorporated herein by reference. Additional non-limiting examples of functional monomers, cross-linkers and initiators that can be used to generate an artificial binding microbe sampling unit are provided. See, e.g., U.S. Pat. No. 7,319,038; Alexander, et al. (2006) J. Mol. Recognit. 19:106-180, each of which is incorporated herein by reference. In an aspect, hydrogels can be used for molecular imprinting. Other examples of synthetic binders are provided. See, e.g., U.S. Pat. Nos. 6,255,461; and 6,797,522; and Ye and Haupt (2004) Anal Bioanal Chem. 378: 1887-1897; Peppas and Huang (2002) Pharm Res. 19: 578-587, each of which is incorporated herein by reference.

In an aspect, at least one of the plurality of specific microbe-binding elements recognizes and binds DNA and/or RNA sequences associated with the at least one type of microbe. For example, cytoplasmic components of the microbes, e.g., RNA and/or DNA, can be made accessible to a specific microbe-binding element by lysing the microbes with a lysing agent, e.g., a detergent. For example, the specific microbe-binding element may be a cDNA element engaged in DNA-DNA hybridization with microbe DNA sequence. In an aspect, the specific microbe-binding element may include oligonucleotides capable of binding to unique 16S small subunit ribosomal (rRNA) genes. In an aspect, various phylogenetic markers may be targeted including ribosomal RNA, elongation and initiation factors, RNA polymerase subunits, DNA gyrases, heat shock proteins, and recA proteins.

In an aspect, the plurality of specific microbe-binding elements are incorporated into the microbe sampling unit. In an aspect, the plurality of specific microbe-binding elements are substantially uniformly distributed throughout the microbe sampling unit. For example, the plurality of specific microbe-binding elements may be uniformly dispersed in a liquid or gelled form during manufacture of the microbe sampling unit.

In an aspect, the plurality of specific microbe-binding elements are substantially distributed along at least one surface of the microbe sampling unit. In an aspect, the plurality of specific microbe-binding elements are functionally attached to at least one surface of the microbe sampling unit. In an aspect, the plurality of specific microbe-binding elements are covalently attached to at least one surface of the microbe sampling unit through amine groups, carbohydrate groups, sulfhydryl groups, or combinations thereof using a homobifunctional, heterobifunctional, and/or photoreactive crosslinking reagent. The plurality of specific microbe-binding elements can be cross-linked to the outer surface of the microbe sampling unit through amine groups, carbohydrate groups, sulfhydryl groups, or combinations thereof associated with a component of the signal-generating complex. A variety of crosslinking reagents are known and available from commercial sources (from, e.g., Pierce-Thermo Fisher Scientific, Inc., Rockford, Ill.). For example, the at least one surface of the microbe sampling unit may include a layer of silane to which is bound one arm of the heterobifunctional crosslinking reagent. The other arm of the heterobifunctional crosslinking reagent may be covalently bound at least one type of specific microbe-binding element. See, e.g., U.S. Pat. No. 5,077,210, which is incorporated herein by reference.

In an aspect, the plurality of specific microbe-binding elements are non-covalently attached to at least one surface of the microbe sampling unit. Non-limiting examples of non-covalent interactions include hydrogen bonds, ionic bonds, van der Waals forces, and hydrophobic interactions. In an aspect, the plurality of specific microbe-binding elements are non-covalently attached to at least one surface of the microbe sampling unit through protein-protein interactions. For example, a type of specific microbe-binding element that includes biotin can be non-covalently attached to at least one surface of the microbe sampling unit including streptavidin or avidin. For example, a single chain antibody may incorporate streptavidin as part of a fusion protein to facilitate attachment of the antibody to an microbe sampling unit via a biotin-streptavidin linkage. See, e.g., Koo et al. (1999) Appl. Environ. Microbiol. 64:2497-2502, which is incorporated herein by reference. Other non-limiting examples non-covalent interactions include interactions between protein A or protein G and immunoglobulins, ligands with receptors, and secondary antibodies with primary antibodies.

### Microbe-Capture Region—Plurality of Signal Generating Complexes

In an aspect, the microbe-capture region of the microbe sampling unit includes a plurality of signal-generating complexes. In general, at least one of the plurality of signal-generating complexes emits a signal, e.g., an optical signal, in response to interaction with at least one type of microbe. In an aspect, at least one of the plurality of signal-generating complexes includes an optical signal-generating complex, a fluorescing signal-generating complex, an electromagnetic signal-generating complex, a radio signal-generating complex, an electrical current signal-generating complex, an acoustic signal-generating complex, or a magnetic signal-generating complex.

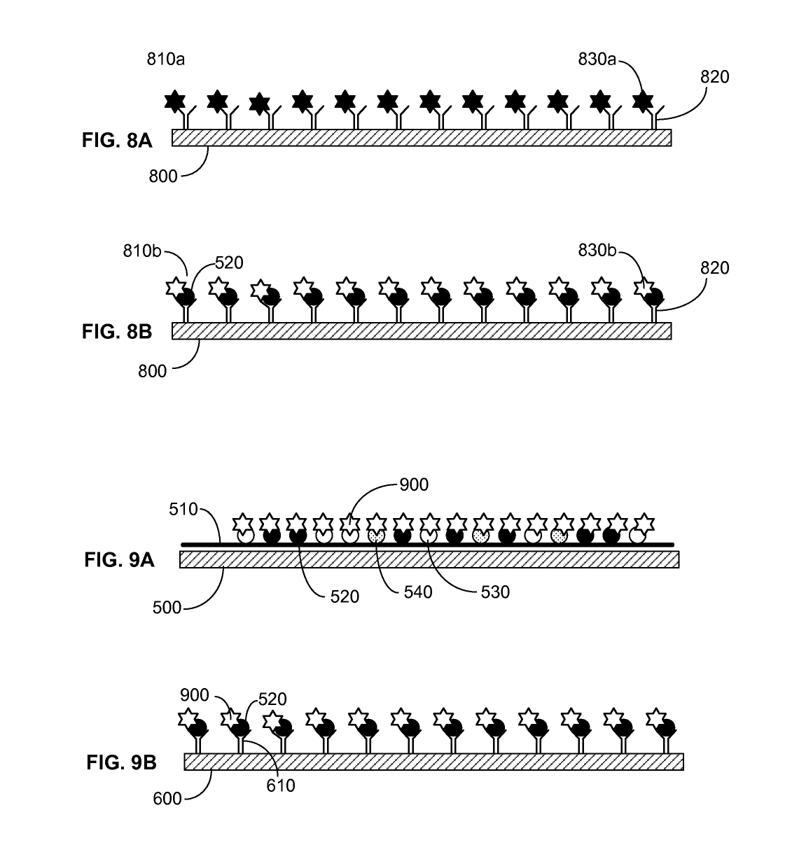

With reference to FIGS. 8A and 8B, shown is at least one surface 800 of a microbe sampling unit with a microbe capture region including a plurality of signal-generating complexes in a first state 810a including at least one specific microbe-binding element 820 operably coupled to at least one signal-generating element in a first state 830a. Interaction of the signal-generating complex with at least one type of microbe causes the complex to signal, e.g., shifting from a first state to a second state. For example, shifting from a first state to a second state can include a shift in detectable color or fluorescence. As an example, FIG. 8B illustrates first type of microbe 520 bound to signal-generating complex in a second state 810b on at least one surface 800 of a microbe sampling unit. Signal-generating complex in a second state 810b includes specific microbe-binding element 820 and signal-generating element in a second state 830b.

In an aspect, the signal-generating complex is incorporated into a surface of the microbe sampling unit. In an aspect, the signal-generating complex can include a responsive material attached to a surface of the microbe sampling unit. For example, the surface of the microbe sampling unit can include a polymer which changes color in response to binding a target, e.g., bacteria. See, e.g., WO2008/059274, which is incorporated herein by reference. In an aspect, the surface of the microbe sampling unit can include a negative chromogen which loses color in response to binding a microbe.

In an aspect, at least one of the plurality of signal-generating complexes associated with the microbe sampling unit is configured to emit one or more signals in response to interaction with at least one type of microbe on a body surface of a user. In an aspect, the interaction with the at least one type of microbe is a binding interaction, in which the at least one type of microbe binds to a portion of the signal-generating complex and induces emission of a signal. In an aspect, the microbe may be physically attached to the signal-generating complex. In an aspect, a brief interaction between a microbe and the signal-generating complex may be sufficient to induce a signal. In an aspect, the interaction of the signal-generating complex with the at least one type of microbe is a chemical interaction, in which some component of the microbe, e.g., an excreted component or metabolite, interacts with the signal-generating complex to induce emission of a signal.

In an aspect, the plurality of signal-generating complexes are incorporated into the microbe sampling unit. In an aspect, the plurality of signal-generating complexes are substantially uniformly distributed throughout the microbe sampling unit. For example, the plurality of signal-generating complexes may be uniformly dispersed in a liquid or gelled form during manufacture of the microbe sampling unit. In an aspect, at least a portion of the plurality of signal-generating complexes are distributed along at least a portion of the outer surface of the microbe sampling unit. In an aspect, the plurality of signal-generating complexes are substantially uniformly distributed over at least a portion of the outer surface of the microbe sampling unit.

In an aspect, at least a portion of the plurality of signal-generating complexes are functionally attached to the outer surface of the microbe sampling unit. In an aspect, at least one of the plurality of signal-generating complexes are covalently attached to the outer surface of the microbe sampling unit. In an aspect, at least one of the plurality of signal-generating complexes is non-covalently attached to the outer surface of the microbe sampling unit.

In an aspect, the microbe-capture region includes a plurality of signal-generating complexes of at least one first type able to emit at least one first signal type in response to at least one first type of microbe and a plurality of signal-generating complexes of at least one second type able to emit at least one second signal type in response to at least one second type of microbe. In an aspect, the plurality of signal-generating complexes of the at least one first type differ from the plurality of signal-generating complexes of the at least one second type. In an aspect, the at least one first type of microbe differs from the at least one second type of microbe. For example, the at least one first type of microbe can include a different phylum from the at least one second type of microbe, e.g., bacteria versus fungi. For example, the at least one first type of microbe can include a different genus from the at least one second type of microbe, e.g., Staphylococcus versus Propionibacterium. For example, the at least one first type of microbe can include a different species from the at least one second type of microbe, e.g., Staphylococcus aureus versus Staphylococcus epidermidis. In an aspect, the at least one first signal type differs from the at least one second signal type. For example, the at least one first signal type can differ in wavelength, e.g., color, from the at least one second signal type. In an aspect, a specific color can be associated with a response to a specific microbe, e.g., a red signal associated with Staphylococcus and a green signal associated with Propionibacterium.

In an aspect, each of the plurality of signal-generating complexes includes at least one signal-generating element operably coupled to at least one specific microbe-binding element, the at least one signal-generating element configured to emit one or more signals in response to contact with at least one type of microbe by the operably coupled at least one specific microbe-binding element. In an aspect, each of the plurality of signal-generating complexes includes at least one signal-generating element, e.g., a chromogenic or fluorogenic signal-generating element, and at least one specific microbe-binding element, e.g., an antibody, aptamer, or oligonucleotide. Non-limiting examples of other specific microbe-binding elements have been described above herein. In an aspect, the at least one signal-generating element emits one or more signals in response to at least one microbe bound to the at least one operably coupled specific microbe-binding element. In an aspect, the signal-generating element emits one or more signals in response to a structural change in the signal-generating complex in the presence of a microbe. In an aspect, the signal-generating element emits one or more signals only when a microbe is bound, e.g., an on/off detection system. Alternatively, the signal-generating element emits a first signal type in the absence of a bound microbe and a second signal type in the presence of a bound microbe, e.g., a change in the color or other property of emitted light. In an aspect, a given type of signal-generating element is operably coupled to a given type of specific microbe-binding element to provide a microbe-specific signal. For example, a first signal-generating element emitting light at a first wavelength band, e.g., red fluorescence, may be operably coupled to a first type of specific microbe-binding element that binds a first type of microbe while a second signal-generating element emitting light at a second wavelength band, e.g., green fluorescence, may be operably coupled to a second type of specific microbe-binding element that binds a second type of microbe, allowing for distinct detection of the first type of microbe versus the second type of microbe.

In an aspect, the microbe sampling unit includes a plurality of signal-generating complexes of at least one first type including at least one signal-generating element of a first type operably coupled to at least one specific microbe-binding element of a first type, the at least one signal-generating element of the first type to emit one or more signals of a first type in response to at least one first type of microbe bound to the operably coupled at least one specific microbe-binding element of the first type, and a plurality of signal-generating complexes of at least one second type including at least one signal-generating element of a second type operably coupled to at least one specific microbe-binding element of a second type, the at least one signal-generating element of the second type to emit one or more signals for a second type in response to at least one second type of microbe bound to the operably coupled at least one specific microbe-binding element of the second type. In an aspect, the at least one first type of microbe differs from the at least one second type of microbe. In an aspect, the one or more signals of the first type differ from the one or more signals of the second type.

In an aspect, the signal-generating element can include any of a number of elements capable of emitting a signal, e.g., an optical, fluorescence, magnetic, electromagnetic, acoustic, radioactive, electrical, or radiofrequency signal. Non-limiting examples of signal-generating elements include optical signal-generating elements, fluorescence signal-generating elements, electrical signal-generating elements, radio signal-generating elements, electromagnetic signal-generating elements, acoustic signal-generating elements, or magnetic signal-generating elements. Non-limiting examples of signal-generating elements include, but are not limited to, at least one of a fluorescent element, an electromagnetic-emitting element, a quantum dot, a gold label, dye, or chemiluminescent dye, or a combination thereof. Non-limiting examples of additional signal-generating elements include at least one of a radioactive element; a radiopaque dye; a radiofrequency identification tag; chromogenic element; a contrast agent, a visible dye, volatile label; mass label; luminescent label, e.g., bioluminescent or chemiluminescent; metallic label, e.g., gold particles, magnetic beads, or paramagnetic beads; dyes, e.g., direct, indirect, or releasable; or a combination thereof.

In an aspect, the signal-generating element includes a chromogenic or fluorogenic signal-generating element. In an aspect, the chromogenic or fluorogenic signal-generating element can be a chemical entity operably coupled to the specific microbe-binding element, so that the chemical entity changes color in response to an interaction with a microbe, e.g., binding the microbe. In an aspect, the chromogenic or fluorogenic signal-generating element can change color in response to metabolism of a microbe bound to and/or in proximity to the outer surface of the skin-covering material. In an aspect, the chromogenic or fluorogenic signal-generating element can change color in response to one or more components excreted from a microbe in proximity to the signal-generating complex. For example, the chromogenic or fluorogenic signal-generating element can by linked to metabolic activity of certain classes of biochemicals including sugars, hexo-phosphates, amino acids, hexose sugars, carboxylic acids, esters, and fatty acids. In an aspect, the chromogenic or fluorogenic signal-generating element can change color in response to an interaction with a microbe independent of the specific microbe-binding element. For example, the chromogenic or fluorogenic signal-generating element can include tetrazolium salts, which form violet-colored formazans in response to microbe metabolism. See, e.g., Tachon et al. (2009) Microbiology 155:2941-2948, which is incorporated herein by reference.

In an aspect, the signal-generating complex includes a chromogenic substrate. Chromogenic substrates can include peptides that generate color in response to interaction with microbe-derived proteolytic enzymes. For example, the chromogenic substrate may include in part a chemical group, e.g., para-nitroaniline, which generates a color change when released by enzymatic cleavage. For example, a chromogenic substrate associated with the outer surface of the microbe sampling unit may interact with an enzyme located on the exterior of the microbe, e.g., located in a bacterial cell wall, to generate a color signal. As an example, L-alanine-4-nitroanilide can be used as a chromogenic substrate for L-alanine-aminopeptidase, commonly associated with Gram-negative bacteria. The substrate L-alanine-4-nitroanilide is split by L-alanine aminopeptidases into L-alanine and 4-nitroaniline, the latter producing a yellow color. The color change can be followed spectrophotometrically and may be proportional to the proteolytic activity.

In an aspect, the signal-generating complex includes a fluorogenic signal-generating complex. In an aspect, fluorogenic signal-generating complex can include chemical dyes or fluorophores that emit light, i.e., fluoresce, at various wavelengths in response to excitation energy. In an aspect, the fluorogenic signal-generating complex can include a quantum dot or semiconductor nanocrystals that fluoresce at various wavelengths in response to excitation energy. In an aspect, the fluorogenic signal-generating complex includes at least one fluorogenic signal-generating element, e.g., a fluorescing dyes, non-limiting examples of which have been described above herein.

In an aspect, the signal-generating complex includes a magnetic signal-generating complex including magnetic beads or particles. In an aspect, the signal-generating complex can include magnetic beads or particles conjugated to the complex via an enzymatically cleavable linkage which in the presence of a microbe is cleaved, releasing the magnetic bead or particle. In an aspect, magnetic beads and magnetic particles of various sub-millimeter size are available from commercial sources (e.g., from Seradyn-Thermo Scientific, Indianapolis, Ind.; Dynal-Invitrogen, Carlsbad, Calif.).

In an aspect, the signal-generating complex includes a radiofrequency identification tag. In an aspect, the signal-generating complex can include a radiofrequency identification tag conjugated to the complex via an enzymatically cleavable linkage, which in the presence of a microbe is cleaved, releasing the radiofrequency identification tag. In an aspect, the signal-generating complex can include a sub-millimeter radiofrequency identification tag. See, e.g., Hornyak (2008) Scientific American Magazine, pp 68-71, February 2008, which is incorporated herein by reference. Alternatively, the signal-generating complex can include one or more bokodes, millimeter sized visual tags that can be captured with a camera. See, e.g., Mohan et al. ACM Transactions on Graphics Proceedings of SIGGRAPH 2009, Aug. 3-7, 2009, New Orleans, which is incorporated herein by reference.

In an aspect, the signal-generating complex can be configured such that binding of one or more microbes to the specific microbe-binding element operably coupled to the signal-generating element results in a conformational change that induces a fluorescence resonance energy transfer (FRET). FRET is a distance-dependent interaction between the electronic excited states of two dye molecules in which excitation is transferred from a donor molecule to an acceptor molecule without emission of a photon. In an aspect, interaction of a donor molecule with an acceptor molecule can lead to a shift in the emission wavelength associated with excitation of the acceptor molecule. In an aspect, interaction of a donor molecule with an acceptor molecule can lead to quenching of the donor emission. In an aspect, the signal-generating complex can include at least one signal-generating element that includes at least one donor molecule and at least one acceptor molecule attached to a specific microbe-binding element, e.g., an antibody or aptamer. In this configuration, interaction of at least one type of microbe with the specific microbe-binding element, e.g., the antibody or aptamer, causes a conformational change in the specific microbe-binding element and results in a change in the distance between the donor and acceptor molecules components of the signal-generating element and a change in measurable signal, e.g., fluorescence.

A variety of donor and acceptor fluorophore pairs can be considered for FRET including, but not limited to, fluorescein and tetramethylrhodamine; IAEDANS and fluorescein; fluorescein and fluorescein; and BODIPY FL and BODIPY FL. A number of Alexa Fluor (AF) fluorophores (Molecular Probes-Invitrogen, Carlsbad, Calif., USA) can be paired with other AF fluorophores for use in FRET. Some examples include, but are not limited, to AF 350 with AF 488; AF 488 with AF 546, AF 555, AF 568, or AF 647; AF 546 with AF 568, AF 594, or AF 647; AF 555 with AF594 or AF647; AF 568 with AF6456; and AF594 with AF 647.

Other non-limiting examples of fluorophores for FRET-based signaling include cyanine dyes Cy3, Cy5, Cy5.5 and Cy7, which emit in the red and far red wavelength range (>550 nm). For example, Cy3, which emits maximally at 570 nm and Cy5, which emits at 670 nm, can be used as a donor-acceptor pair. When Cy3 and Cy5 are not proximal to one another, excitation at 540 nm results only in the emission from of light from Cy3 at 590 nm. In contrast, when Cy3 and Cy5 are brought into proximity by a conformation change, e.g., by binding of a microbe to a specific microbe-binding element, excitation at 540 nm results in an emission at 680 nm.

In an aspect, the signal-generating complex includes a quenching dye to quench the fluorescence of visible light-excited fluorophores. Non-limiting examples of quenching dyes include DABCYL, the non-fluorescing diarylrhodamine derivative dyes QSY 7, QSY 9 and QSY 21 (Molecular Probes, Carlsbad, Calif., USA), the non-fluorescing Black Hole Quenchers BHQ0, BHQ1, BHQ2, and BHQ3 (Biosearch Technologies, Inc., Novato, Calif., USA) and Eclipse (Applera Corp., Norwalk, Conn., USA). Non-limiting examples of donor fluorophore and quencher pairs include fluorescein with DABCYL; EDANS with DABCYL; or fluorescein with QSY 7 and QSY 9. For example, QSY 7 and QSY 9 dyes can be used to quench the fluorescence emission of donor dyes including blue-fluorescent coumarins, green- or orange-fluorescent dyes, and conjugates of the Texas Red and Alexa Fluor 594 dyes. Non-limiting examples of fluorophores and quenching molecules are known and commercially available (from, e.g., Molecular Probes-Invitrogen, Carlsbad, Calif., USA).

In an aspect, the signal-generating complex for FRET-based signaling includes a specific microbe-binding element that is an RNA or DNA oligonucleotide-based aptamer and a signal-generating element that includes one or more donor fluorophore and one or more acceptor fluorophore or quencher. See, e.g., Cao et al. (2005) Current Proteomics 2:31-40 and U.S. Patent Application 2009/0186342, which are incorporated herein by reference. For example, the aptamer including a donor fluorophore and an acceptor fluorophore or quencher can be configured to undergo a conformational change upon binding a target, e.g., a microbe, causing the distance between the donor fluorophore and the acceptor fluorophore or quencher to shift and leading to a change in measurable fluorescence. See, e.g., Ikanovic et al. (2007) J. Fluorescence 17:193-199; Jhaveri, et al. (2000) Nature Biotech. 18:1293-1297, which are incorporated herein by reference. The fluorophores can be attached to various linkers that allow for attachment at various sites within the aptamer. For example, 3-prime-DABCYL CPG can be used to place the fluorophore DABCYL at the 3-prime terminus of an aptamer whereas 5-prime-DABCYL phosphoramidite can be used to place DABCYL at the 5-prime terminus of an aptamer (see, e.g., product information at Glen Research, Sterling, Va.). DABCYL deoxythymidine (dT) can be used to place DABCYL within the body of an aptamer sequence. Modifying aptamers with appropriate commercially available fluorophores can be achieved following instructions provided by the respective manufacturer. Alternatively, custom made aptamer-based signaling complexes are available from commercial sources (from, e.g., Biosearch Technologies, Inc., Novato, Calif., USA).

In an aspect, an aptamer-based signal-generating complex includes a semiconductor quantum dot (QDs). Various methods are available for attaching quantum dots to the DNA backbone of an aptamer such as, for example, covalent linkage of amine-modified DNA to carboxylated quantum dots and linkage of biotinylated DNA to streptavidin modified quantum dots. See, e.g., Cady, et al. (2007) J. Mol. Cell. Probes 21:116-124, which is incorporated herein by reference. For example, carboxy quantum dots (from, e.g., Quantum Dot Corporation, Hayward, Calif., USA) can be attached to an aptamer through a C6 amino modifier placed on either the 5-prime or 3-prime end of the aptamer sequence. For example, streptavidin quantum dots (from, e.g., Quantum Dot Corporation, Hayward, Calif., USA) can be attached to an aptamer through a biotin attached to the 5-prime end of the aptamer sequence.

In an aspect, the signal-generating complex for FRET-based signaling includes a specific microbe-binding element that is an antibody configured to bind at least one type of microbe and a signal-generating element that includes one or more donor fluorophore and one or more acceptor fluorophore or quencher. For example, the antibody including a donor fluorophore and an acceptor fluorophore or quencher can be configured to undergo a conformational change upon binding a target, e.g., a microbe, causing the distance between the donor fluorophore and the acceptor fluorophore or quencher to shift, the shift leading to a change in measurable fluorescence. See, e.g., Dwarakanath et al. (2004) Biochem. Biophys. Res. Commun. 323:739-743; Brennan (1999) J. Fluor. 9:295-312, which are incorporated herein by reference. In an aspect, the antibody is modified with a fluorescence signal-generating element such that binding of the target microbe to the antibody shields a solvent sensitive fluorescence signal-generating element near the active binding site from a solvent, e.g., water, resulting in a 3-5 fold increase in fluorescence intensity. See, e.g., Bright, et al. (1990) Anal. Chem. 62:1065-1069, which is incorporated herein by reference.

In an aspect, the signal-generating complex can be configured such that binding of one or more microbes to the specific microbe-binding element operably coupled to the signal-generating element results in a conformational change that can be measured using chemiluminescence resonance energy transfer (CRET). In an aspect, the image-capture device is able to detect luminescence. For example, the interaction of luminol with hydrogen peroxide in the presence of iron or copper and enhanced by horseradish peroxidase results in emitted light. See, e.g., Freeman et al. (2011) J. Am. Chem. Soc. 133:11597-11604; Lee et al. (2012) ACS Nano 6:2978-2983, which are incorporated herein by reference.

In an aspect, the at least one specific microbe-binding element of the signal-generating complex is chemically coupled to the at least one signal-generating element. In an aspect, the specific microbe-binding element and the signal-generating element are directly associated with one another through chemical cross-linking, non-covalent linking, or synthesis as a single molecule. For example, the signal-generating element may be operably coupled to the specific microbe-binding element through one or more of a chemical cross-link, a streptavidin/biotin interaction, a fusion protein construct, a common microbe sampling unit, or a combination thereof.

In an aspect, the signal-generating element is conjugated to the specific microbe-binding element using one or more of a cross-linking agent, non-limiting examples of which have been describe above herein. In general, any of a number of cross-linking agents can be used to conjugate an appropriately derivatized signal-generating element to an appropriately derivatized or functionalized specific microbe-binding element. For example, a fluorescent dye, e.g., rhodamine, derivatized with succinimidyl ester (from, e.g., Invitrogen, Carlsbad, Calif.) will react efficiently with primary amines of proteins, e.g., antibodies, to generate a stable fluorescent dye-protein conjugate. As another example, amine-derivatized, poly-ethylene glycol coated quantum dots can be cross-linked to an antibody via an amine-thiol crosslinker SMCC using a commercially available kit (Qdot® Antibody Conjugation Kit, Invitrogen, Carlsbad, Calif.). Similarly, various methods are available for attaching quantum dots to a DNA backbone of an aptamer such as, for example, covalent linkage of amine-modified DNA to carboxylated quantum dots. For example, carboxy quantum dots (from, e.g., Quantum Dot Corporation, Hayward, Calif., USA) can be attached to an aptamer through a C6 amino modifier placed on either the 5-prime or 3-prime end of the aptamer sequence. Magnetic beads derivatized with carboxylic acid, amine groups or tosylactivated for cross-linking to proteins and appropriately derivatized oligonucleotides are also commercially available (from, e.g., Dynal Biotech, Brown Deer, Wis.). Quantum dots, fluorescent dyes, and magnetic particles derivatized for cross-linking to antibodies, aptamers or other biomolecules are available from a number of commercial sources (from, e.g., Invitrogen, Carlsbad, Calif.; Seradyn-Thermo Scientific, Indianapolis, Ind.; Sigma-Aldrich, St. Louis, Mo.).

In an aspect, the signal-generating element can be incorporated into the specific microbe-binding element at the time of synthesis. In an aspect, the signal-generating complex can include a fusion protein with a specific microbe-binding element, e.g., antibody, peptide ligand, or receptor, and a signal-generating element including all or part of green fluorescent protein (GFP) derived from Aequorea victoria jellyfish or yellow, red and blue fluorescing derivatives thereof. A number of expression constructs for generating recombinant GFP fusion proteins are available from commercial sources (from, e.g., Invitrogen, Carlsbad, Calif.).

In an aspect, the plurality of signal-generating complexes associated with the outer surface of the microbe sampling unit are incorporated into a field effect transistor (FET) based biosensor, in which a change in electrical signal is used to detect interaction of one or more microbes with one or more of the plurality of signal-generating complexes. See, e.g., U.S. Pat. No. 7,303,875, which is incorporated herein by reference. In an aspect, the one or more electrical signals are processed to generate one or more optical signals using light-emitting diodes or semiconductor optical amplifier, the one or more optical signals detectable by the at least one sensor component. In an aspect, the signal-generating complex can include carbon nanotubes functionalized with a specific microbe-binding element. See, e.g., Zelada-Guillen, et al., (2009) Angew. Chem. Int. Ed., 48:7334-7337, which is incorporated herein by reference. Single walled carbon nanotubes can act as efficient ion-to-electron transducers in potentiometric analysis. The carbon nanotubes can be functionalized with a specific microbe-binding element, e.g., an oligonucleotide aptamer. Upon microbe binding to the aptamer, the aptamers change conformation, separating the phosphate groups of the aptamer from the side-walls of the carbon nanotubes and inducing a charge change to the carbon nanotube and recorded potential.

In an aspect, the signal-generating complex can include one or more microcantilevers configured to detect changes in cantilever bending or vibrational frequency in response to binding of one or more microbes to the surface of the microcantilever. In an aspect, the outer surface of the microbe sampling unit can include a plurality of biochips including microcantilever bi-material formed from gold and silicon, as sensing elements. See, e.g. Vashist (2007) J. Nanotech Online 3:DO: 10.2240/azojono0115, which is incorporated herein by reference. The gold component of the microcantilever can be functionalized with one or more specific microbe-binding elements, e.g., aptamer, antibodies, or other microbe binding elements. A number of microcantilever deflection detection methods can be used to measure microbe binding including, among other things, optical deflection detection, interferometry deflection detection, optical diffraction grating deflection detection, and charge coupled device detection. In some aspects, the one or more microcantilever can be a nanocantilever with nanoscale components. The one or more microcantilevers and/or nanocantilevers can be arranged into arrays for detection of one or more target cells. Both microcantilevers and nanocantilevers can find utility in microelectromechnical systems (MEMS) and/or nanoelectromechnical systems (NEMS).

In an aspect, the signal-generating complex includes label-free optical biosensors that incorporate other optical methodologies, e.g., interferometers, waveguides, fiber gratings, ring resonators, and photonic crystals. See, e.g., Fan, et al., Anal. Chim. Acta 620:8-26, 2008, which is incorporated herein by reference.

### Adding Signal-Generating Elements to Captured Microbes

In an aspect, at least one type of signal-generating element is added to the microbe-capture region of the microbe sampling unit following capture of one or more types of microbes. In an aspect, the kit described herein includes at least one type of signal-generating element to be added to the surface of the microbe sampling unit following capture of one or more types of microbes from a body surface of the user prior to analyzing the microbe-capture region with the analyzer. For example, a microbe sampling unit can be exposed to at least one of a powder, liquid, gel, or spray containing at least one type of signal-generating element to allow for detection of the one or more types of microbes captured by the microbe sampling unit. Non-limiting examples are illustrated in FIGS. 9A and 9B. FIG. 9A shows at least one surface 500 of a microbe sampling unit with microbe-capture region 510 and captured thereto first type of microbe 520, second type of microbe 530, and third type of microbe 540. In addition, signal-generating element 900 is shown associated with each of the captured microbes. In an aspect, signal-generating element 900 binds non-selectively to all microbes captured on microbe-capture region 510. In an aspect, signal-generating element 900 binds selectively to a subset of microbes captured on microbe-capture region 510. FIG. 9B shows at least one surface 600 of a microbe sampling unit including a microbe-capture region with a plurality of specific microbe-binding elements 610 and captured thereto first type of microbe 520. In addition, signal-generating element 900 is shown associated with each of the captured microbes.

In an aspect, the added signal-generating element includes at least one element that emits or reflects a signal, e.g., an optical signal, fluorescence signal, electromagnetic signal, magnetic signal, electrical signal, radioactive signal, or radiofrequency signal. A number of non-limiting examples of signal-generating elements have been described above herein. In a further aspect, the signal-generating element can include an ink, stain, or dye that emits or reflects ultraviolet, near infrared, or infrared electromagnetic energy. In an aspect, the signal-generating element can include one or more histological stain, non-limiting examples of which include crystal violet, safranin, fuschin, methylene blue, or Giemsa stain. In an aspect, the signal-generating element can include a differential stain, e.g., a Gram's stain, which uses crystal violet with the mordant Gram's iodine and a counterstain, or an acid-fast stain. In an aspect, the signal-generating element can include a non-selective vital dye, e.g., a redox stain, e.g., 5-cyano-2,3-ditolyl tetrazoliumchloride (CTC). In an aspect, the signal-generating element includes a vital dye that intercalates into nucleic acids of microbes, non-limiting examples of which include DAPI (4′,6-diamidino-2-phenylindole), acridine orange, or Hoechst stain. Other non-limiting examples of vital dyes include calcein AM, carboxyfluorescein diacetate, DiOC (3,3′-dihexyloxacarbocyanine iodide), rhodamine 123, and Nile red. In an aspect, the signal-generating element can include a stain that will react with a polysaccharide, non-limiting examples of which include Schiff's reagent or a diamino stilbene, e.g., Calcofluor (from, e.g., Polysciences, Inc., Warrington, Pa.). In an aspect, the signal-generating element can include a negative stain, e.g., India ink or nigrosin, which stains the area surrounding the captured microbes, but not the microbes.

In an aspect, the signal-generating element can include a dye-labeled antibody, aptamer, or other binding agent that recognizes at least one type of microbe captured on the microbe-capture region. For example, the dye-labeled antibody, aptamer, or other binding agent can bind to one or more biomolecule exposed on the outer surface of a microbe, e.g., a protein, carbohydrate or lipid biomolecule exposed on the outer surface of the microbe. The label associated with the antibody, aptamer, or other binding agent can include a fluorescent label, a colored label, or a chemiluminescent label. For example, the labeled antibody, aptamer, or other binding agent configured to bind the at least one type of microbe may further include fluorescein for direct fluorescence detection or horseradish peroxidase (HRP) for indirect detection using colorimetric or chemiluminesence following addition of peroxidase substrate. In some embodiments, the labeled antibody, aptamer, or other binding agent configured to bind the at least one type of microbe may further include biotin conjugates available for binding with avidin or streptavidin. Other non-limiting examples of signal-generating elements include labeled oligonucelotides, anti-16S rRNAs, antibody fragments, peptides, protein nucleic acids, proteins, viruses, lipids, phospholipids, carbohydrates, enzymes, receptors, lectin, peptide aptamer, bacteria, cells, cell fragments, inorganic molecules, organic molecules, synthetic ligands, artificial binding substrates, mimetic binding elements (e.g., formed by molecular imprinting), or combinations thereof.

In an aspect, the signal-generating element includes at least one fluorescence-generating element. In an aspect, the signal-generating element includes at least one chemiluminescence-generating element. In an aspect, the signal-generating element includes an anti-16S RNA labeled with a chromophore or fluorophore. In an aspect, the signal-generating element includes universal primers of the type used for amplification of microbial 16S gene sequencing the 1.4 kb amplicon and comparing with known sequences in a database. See, e.g., references regarding Ribosomal Database Project (Cole et al. (2009) Nucl. Acids Res. 37(D1):D141-D145); SILVA (Quast et al. (2013) Nucl. Acids Res. 41(D1):D590-D596); CORE (“core human oral microbiome;” Griffen et al. (2011), PLoS ONE 6(4):e19051), which are incorporated herein by reference. Other non-limiting examples of signal-generating elements include radioactive agents, magnetic agents, radiofrequency identification tags, or contrast agents.

In an aspect, the signal-generating element can include a chromogenic, fluorogenic, or luminescent substrate. Chromogenic substrates can include peptides that react with microbe-derived proteolytic enzymes under the formation of color. For example, the chromogenic substrate may include a chemical group which when released after enzyme cleavage gives rise to color. The color change can be followed spectrophotometrically and may be proportional to the proteolytic activity. For example, the fluorogenic substrate may include a chemical group including a fluorophore, which, when released after enzymatic cleavage or chemical reaction, is fluorescent. For example, a chemiluminescent substrate may include a chemical group which when released after enzyme cleavage or chemical reaction produces light.

In an aspect, the signal-generating element is a fluorescence signal-generating element. In an aspect, fluorescence signal-emitting elements can include chemical dyes that emit light, i.e., fluoresce, at various wavelengths in response to an excitation energy. In an aspect, the fluorescence signal-generating element can include a quantum dot or semiconductor nanocrystals that fluoresce at various wavelengths in response to an excitation energy. See, e.g., Jaiswal et al. (2003) Nature Biotech. 21:47-51, which is incorporated herein by reference. Non-limiting examples of fluorescing dyes include fluorescein (FITC), indocyanine green (ICG) and rhodamine B, red and near infrared emitting fluorophores (600-1200 nm) including cyanine dyes such as Cy5, Cy5.5, and Cy7 (Amersham Biosciences, Piscataway, N.J., USA) and/or a variety of Alexa Fluor dyes such as Alexa Fluor 633, Alexa Fluor 635, Alexa Fluor 647, Alexa Fluor 660, Alexa Fluor 680, Alexa Fluor 700 and Alexa Fluor 750 (Molecular Probes-Invitrogen, Carlsbad, Calif., USA). Additional fluorophores include IRDye800, IRDye700, and IRDye680 (LI-COR, Lincoln, Nebr., USA), NIR-1 and 105-OSu (Dejindo, Kumamotot, Japan), LaJolla Blue (Diatron, Miami, Fla., USA), FAR-Blue, FAR-Green One, and FAR-Green Two (Innosense, Giacosa, Italy), ADS 790-NS and ADS 821-NS (American Dye Source, Montreal, Calif.), NIAD-4 (ICx Technologies, Arlington, Va.). Other fluorescing dyes include BODIPY-FL, europium, green, yellow and red fluorescent proteins, luciferase.

In an aspect, the signal-generating element can include a magnetic marker, e.g., magnetic beads, magnetic particles or carbon nanotubes. Magnetic beads and magnetic particles of various sub-millimeter size are available from commercial sources (e.g., from Seradyn-Thermo Scientific, Indianapolis, Ind.; Dynal-Invitrogen, Carlsbad, Calif.). Carbon nanotubes with various functionalities can be synthesized de novo (see, e.g., Didenko & Baskin (2006) BioTechniques 40:295-302, which is incorporated herein by reference) or may be available from commercial sources (e.g., from Nanolab, Newton, Mass.; Swan Chemical Inc., Lyndhurst, N.J.).

In an aspect, the signal-generating element can include a radiofrequency identification (RFID) tag, sub-millimeter versions of which have been described. See, e.g., Hornyak Scientific American Magazine, pp 68-71, February 2008, which is incorporated herein by reference. Alternatively, the signal-generating agent can include one or more bokodes, millimeter sized visual tags that can be captured with a camera. See, e.g., Mohan et al. ACM Transactions on Graphics Proceedings of SIGGRAPH 2009, Aug. 3-7, 2009, New Orleans, which is incorporated herein by reference.

In an aspect, the signal-generating element includes paramagnetic and supramagnetic agents with one or more unpaired electrons, e.g., manganese, iron, or gadolinium, for use in magnetic imaging.

In an aspect, a plurality of signal-generating elements are applied to a body surface prior to capturing one or more types of microbes from the body surface with the microbe sampling unit. For example, a fluorescence-labeled antibody that specifically recognizes and binds to one or more types of microbes on a body surface can be applied to the body surface prior to capturing the one or more types of microbes from the body surface with the microbe sampling unit. In an aspect, the plurality of signal-generating elements are applied to a body surface prior to using a microbe sampling unit that includes at least one optical sensor.

### Analyzer

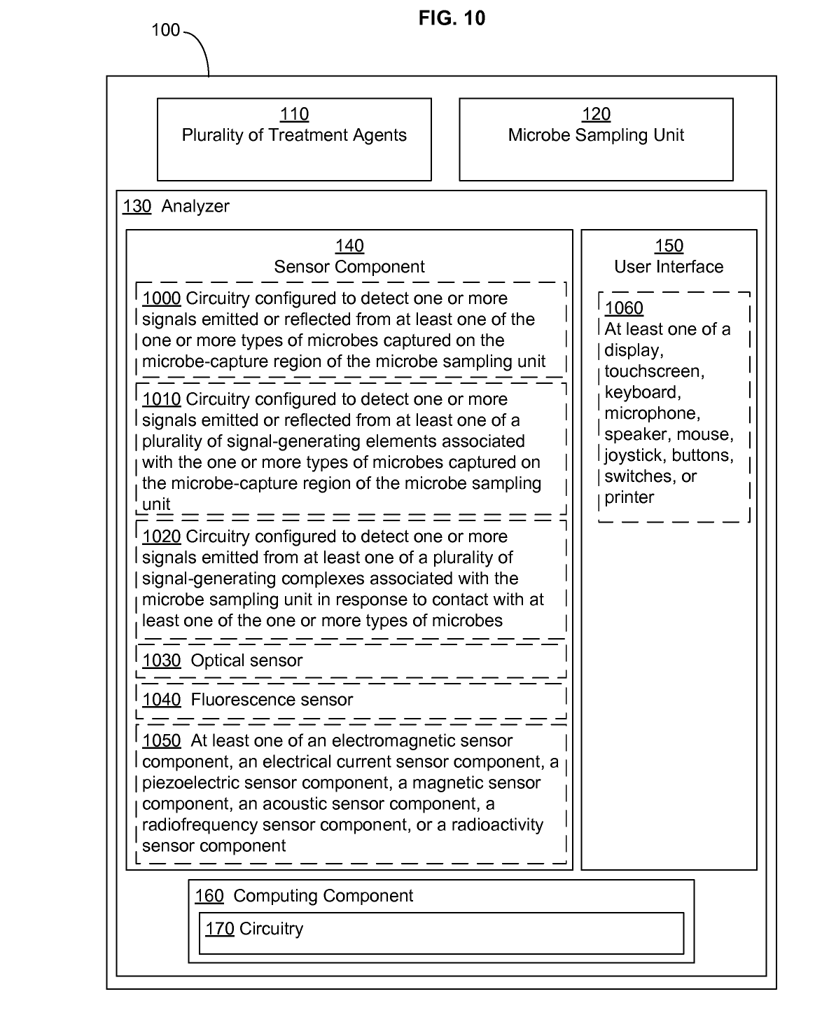

A kit such as shown in FIG. 1 includes analyzer 130 for detecting one or more signals emitted or reflected from the microbe-capture region of microbe sampling unit 120 and translating the detected signals into a microbe profile. In an aspect, analyzer 130 includes at least one sensor component 140 including circuitry configured to detect one or more signals emitted or reflected from one or more microbes captured on the microbe-capture region of microbe sampling unit 120 and to transform the detected one or more signals into a sensor output. FIG. 10 illustrates further aspects of the at least one sensor component 140. In an aspect, at least one sensor component of analyzer 130 includes circuitry 1000 configured to detect one or more signals emitted or reflected from at least one of the one or more types of microbes captured on the microbe-capture region of the microbe sampling unit and to transform the detected one or more signals into a signal output. In an aspect, the one or more signals emitted or reflected from the at least one type of microbe captured on the microbe-capture region are representative of one or more properties of the at least one type of microbe. The one or more properties can include one or more inherent properties or characteristics of the at least one type of microbe that are measureable by the at least one sensor component. In an aspect, the one or more properties of the at least one type of microbe can include at least one of an optical property, an autofluorescence property, an infrared spectral property, a reflective property, a light scattering property, or an opacity property of the at least one type of microbe. In an aspect, the one or more properties of the at least one type of microbe include at least one of metabolic properties, lipid properties, carbohydrate properties, protein properties, or genomic properties of the at least one type of microbe.

In an aspect, analyzer 130 includes at least one sensor component 140 including circuitry 1010 configured to detect one or more signals emitted or reflected from at least one of a plurality of signal-generating elements associated with the one or more types of microbes captured on the microbe-capture region of the microbe sampling unit and to transform the detected one or more signals into a signal output. In an aspect, the one or more signals detected from the at least one of the plurality of signal-generating elements includes optical signals, fluorescent signals, electrical signals, electromagnetic signals, acoustic signals, radioactive signals, magnetic signals, or radio signals.

In an aspect, analyzer 130 includes at least one sensor component 140 including circuitry 1020 configured to detect one or more signals emitted from at least one of a plurality of signal-generating complexes associated with the microbe sampling unit in response to contact with at least one of the one or more types of microbes. In an aspect, the one or more signals detected from the at least one of the plurality of signal-generating complexes includes optical signals, fluorescent signals, electrical signals, electromagnetics signals, acoustic signals, radioactive signals, magnetic signals, or radio signals.

In an aspect, the at least one sensor component includes an energy-emitting mechanism and circuitry configured to scan a surface of the microbe sampling unit to detect one or more signals emitted or reflected from one or more microbes captured on the microbe-capture region of the microbe sampling unit. For example, the at least one sensor component can include an electromagnetic energy source, e.g., a laser, that emits a wavelength of light that causes autofluorescence of the one or more microbes captured on the microbe-capture region. In an aspect, the at least one sensor component includes an energy emitting mechanism and circuitry configured to scan a surface of the microbe sampling unit to detect one or more signals emitted or reflected from at least one type of signal-generating element associated with at least one type of microbe on the microbe-capture region and/or microbe sampling unit. For example, the at least one sensor component can include an electromagnetic energy source, e.g., a laser, that emits a wavelength of light that causes a fluorescence-generating element associated with the at least one type of microbe to fluoresce.

In an aspect, the at least one sensor component can be configured to measure light absorption, light emission, fluorescence, luminescence, chemiluminescence, or phosphorescence associated with the one or more types of microbes or signal-generating elements associated with the one or more types of microbes. Such electromagnetic properties can be inherent properties of all or a portion of the type of microbe (e.g. auto-fluorescence), or can be associated with one or more signal-generating elements incorporated into or added to the microbe-capture region on the microbe sampling unit or the captured one or more types of microbes.

Returning to FIG. 10, in an aspect, at least one sensor component 140 includes at least one optical sensor 1030. In an aspect, the at least one optical sensor includes an image-capture device, e.g., a camera such as a digital camera, configured to capture one or more images of the microbe sampling unit and associated microbe-capture region. In an aspect, the at least one camera may capture one or more images of the microbe sampling unit in the visible spectrum. In an aspect, the at least one camera may capture one or more images of the microbe sampling unit in other portions of the electromagnetic spectrum, e.g., infrared or ultraviolet. In an aspect, the at least one camera may capture emitted and/or reflected light. In an aspect, the at least one sensor component includes one or more electronic image sensors, e.g., photodiodes, photoresistors, charge-coupled devices (CCD), and/or complementary metal oxide semiconductor (CMOS) devices. In an aspect, the at least one sensor component includes a single-shot capture device with one CCD with a Bayer filter mosaic or three separate image sensors, which are exposed to the same image via a beam splitter. In an aspect, the at least one sensor component includes a multi-shot capture device. For example, a single CCD sensor may obtain additive color information by capturing an image three times, each with a different filter (e.g., red, green, and blue). In an aspect, the at least one sensor component includes components for micro-scanning in which a single CCD sensor with a Bayer filter is moved over the focus plane of the lens to “stitch” together a higher resolution image than the CCD would allow otherwise. In an aspect, the micro-scanning device includes a micro laser scanning device. See, e.g., Seidl et al. (2006) International Society for Photogrammetry and Remote Sensing. Volume XXXVI Part 5. Sep. 25-27, 2006, Dresden Germany. In an aspect, the image sensor can include an area array of CCD or CMOS sensors. In an aspect, the image sensor can include a linear array of CCD (monochrome) or 3-strip CCD with color filters. In an aspect, the at least one sensor component includes a lens-free imaging system. See, e.g., Kim et al. (2012) J. Lab. Automation 17:43-49, which is incorporated herein by reference.

In an aspect, the at least one sensor component includes at least one scanning device. Non-limiting examples of scanners include optical scanners, fluorescence scanners, acoustic scanners, electrical scanners, electromagnetic scanners, or magnetic scanners. In an aspect, the scanner includes an energy-emitting mechanism, e.g., a light source or a laser, and circuitry configured to scan the surface of the microbe sampling unit with directed energy, e.g., light of a specified wavelength, to detect one or more signals emitted or reflected from the surface of the microbe sampling unit and to transform the one or more detected signals into a sensor output.

In an aspect, the at least one sensor component includes a colorimetric scanner configured to detect a reflective property, e.g., color, of at least one of the captured one or more types of microbes or a colored reagent in proximity to the captured one or more types of microbes. For example, the color may arise from addition of one or more developing reagents, e.g., a chromogenic antibody or chemically modified antibody, e.g., alkaline phosphatase- or horseradish peroxidase-modified antibody, capable of undergoing a colorimetric change, or a stain or dye able to directly apply color to a microbe or to the microbe sampling unit. In an aspect, the colorimetric scanner includes a camera or other image-capture device.

In an aspect, the at least one sensor component measures changes in refractive index on a surface of the microbe sampling unit. For example, the surface of the microbe sampling unit can be illuminated with a light source at various angles and resonance occurring at specific angles measured to detect the presence of the microbes on the surface. See, e.g., Barlen, et al. (2007) Sensors, 7:1427-1446; and Kashyap & Nemova (2009) J. Sensors: Article ID 645162, each of which is incorporated herein by reference.

In an aspect, the at least one sensor component includes a darkfield scanner capable of scanning an optical pattern of microbes, e.g., bacteria on a solid surface. See, e.g., Adak et al. (2010) Bioconjug. Chem. 21:2065-2075, which is incorporated herein by reference.

In an aspect, at least one sensor component 140 includes at least one fluorescence sensor 1040. In an aspect, the at least one fluorescence sensor includes at least one fluorescence scanning device. In an aspect, the fluorescence scanning device can include a light source that delivers light of fixed excitation/emission wavelengths based on the use of standard commercially available fluorescent dyes in the green, red, and near infrared wavelengths. For example, the fluorescence scanning device can include a two color scanner for scanning at least two distinct wavelengths or wavelength bands. In an aspect, the fluorescence scanning device can include a light source that delivers light of adjustable excitation/emission wavelengths, e.g., with one or more excitation sources and filters to adjust the excitation/emission wavelengths.

In an aspect, the at least one fluorescence sensor includes circuitry configured to detect one or more signals associated with fluorescence emitted from at least one of a plurality of fluorescence signal-generating elements associated with one or more types of microbes captured on the microbe-capture region of the microbe sampling unit in response to a directed energy, e.g., light of a specific wavelength, applied to the microbe-capture region of the microbe sampling unit. For example, the at least one fluorescence sensor, e.g., a photodiode, can include circuitry configured to detect one or more signals emitted from a fluorescing antibody, e.g., a fluorescein-labeled antibody, bound to one or more types of microbes captured on the microbe-capture region of the microbe sampling unit.

In an aspect, the at least one fluorescence sensor includes circuitry configured to detect one or more signals associated with autofluorescence emitted from one or more types of microbes captured on the microbe-capture region of the microbe sampling unit in response to a directed energy, e.g., light of a specific wavelength, applied to the microbe-capture region of the microbe sampling unit. For example, naturally occurring autofluorescence emitted by microbes may be derived from fluorophore-containing biomolecules associated with the microbes, e.g., porphyrins, certain amino acids, flavins, and coenzymes NADP and NADPH (see, e.g., Koenig et al. (1994) J. Fluoresc. 4:17-40, which is incorporated herein by reference). In an aspect, a fluorescence scanning device can include directed energy that includes one or more excitation wavelengths for exciting autofluorescence emission from captured microbes. For example, the excitation maxima of endogenous fluorophores, e.g., porphyrins, lie in the range of 250-450 nm (spanning the ultraviolet/visible (UV/VIS) spectral range), whereas their emission maxima lie in the range of 280-540 nm (see, e.g., Ammor (2007) J. Fluoresc. 17:455-459, which is incorporated herein by reference). See, e.g., U.S. Patent Application 2011/0117025, which is incorporated herein by reference.

In an aspect, the at least one sensor component detects autofluorescence associated with naturally occurring, endogenous prophyrins of bacteria. For example, a number of skin-associated bacteria produce protophorphyrins, including Propinibacterium acnes, Staphylococcus aureus, Clostridium, Bifidobacterium, and Actinomyces (see, e.g., Koenig et al. (1994) J. Fluoresc. 4:17-40, which is incorporated herein by reference). In an aspect, bacteria may be detected using fluorescence lifetimes measured at 430, 487, and 514 nm after selective excitation at 340, 405, and 430 as described by Bouchard et al. (2006) in J. Biomed. Opt. 11:014011, 1-7, which is incorporated herein by reference. In another example, autofluorescence may be used to detect Staphylococcus sp. and/or Pseudomonas aeruginosa using a scanning device emitting electromagnetic energy at a wavelength of 488 nm as described by Hilton (1998) SPIE 3491:1174-1178, which is incorporated herein by reference. For example, Staphylococcus aureus may be distinguished from Escherichia coli based on emission spectra induced by excitations at 410-430 nm (see, e.g., Giana et al. (2002) J. Fluoresc. 13:489-493, which is incorporated herein by reference).

In an aspect, the at least one sensor component detects autofluorescence associated with fungi. For example, Candida albicans irradiated with electromagnetic energy at wavelengths of 465-495 nm autofluoresces at an emission wavelength of 515-555 mm (see, e.g., Mateus et al. (2004) Antimicrob. Agents Chemother. 48:3358-3336, which is incorporated herein by reference). For example, Aspergillus may be detected using autofluorescence in response to excitation at 450-490 nm and emission at 560 (see, e.g., Graham (1983) Am. J. Clin. Pathol. 79:231-234, which is incorporated herein by reference).

In an aspect, the at least one sensor component differentiates autofluorescence associated with different types of microbes, e.g., bacteria versus fungi. For example, bacteria, e.g., Lactobacillus, and fungi, e.g., Saccharomyces, can be differentiated using fluorescence spectroscopy, each having its own spectral fingerprint. See, e.g., Bhatta et al. (2006) Appl. Microbiol. Biotechnol. 71:121-126, which is incorporated herein by reference. For example, a number of skin associated fungi, e.g., dermatophytosis and tinea, exhibit autofluorescence. See, e.g., Elston (2001) BMC Microbiology 1:21, which is incorporated herein by reference.

The at least one sensor component configured to detected one or more signals associated with autofluorescence of one or more types of microbes can include a photosensor such as, for example, a charge coupled device (CCD) and/or a complementary metal oxide semiconductor (CMOS) sensor. The autofluorescence signals are transformed into a sensor output including information associated with at least one property of the autofluorescence signals. The at least one property of the autofluoresence signals, e.g., the emitted wavelength, is compared with preset algorithms defining, for example, the autofluorescence properties of reference microbes.

In an aspect, the at least one sensor component is able to detect chemiluminescence, e.g., light, emitted from at least one type of microbe or signal-generating element on the surface of the microbe sampling unit as a result of a chemical reaction. For example, a chemiluminscent response, i.e., emitted light, may be generated using horseradish peroxidase associated with a specific microbe-binding element, e.g., an antibody or aptamer, in the presence of luminol, hydrogen peroxide, and iron or copper. Chemiluminescence on a solid microbe sampling unit can be detected using a CCD camera system (e.g., GeneGnome5, Syngene USA, Fredrick Md.).

In an aspect, the at least one sensor component includes a confocal laser scanner. In an aspect, the confocal laser scanner includes a MEMS confocal laser scanner. See, e.g., Murakami et al. (2003) The 12^{th} International Conference on Solid State Sensors, Actuators and Microsystems, Boston, Jun. 8-12, 2003, pp. 587-590, which is incorporated herein by reference.

In an aspect, the at least one sensor component includes a spectrometer or spectrophotometer. In an aspect, the spectrophotometer includes a fiber optic spectrophotometer (from, e.g., Ocean Optics, Dunedin Fla.). In an aspect, the sensor component includes a means of vibrational spectroscopy. Examples of vibrational spectroscopy include, but are not limited to, Fourier transform infrared (FTIR) spectroscopy and micro-Raman spectroscopy. Raman spectroscopy can further include UV-resonance Raman spectroscopy, surface enhanced Raman scattering, or tip-enhanced Raman scattering. See, e.g., Harz et al. (2009) Cytometry A 75:104-113, which is incorporated herein by reference.

In an aspect, at least one sensor component 140 includes in block 1050 of FIG. 10 at least one of an electromagnetic sensor component, an electrical current sensor component, a piezoelectric sensor component, a magnetic sensor component, an acoustic sensor component, a radiofrequency sensor component, or a radioactivity sensor component. In an aspect, the at least one sensor component includes one or one or more piezo transducers, one or more MEMS device, one or more cavity resonators, one or more magneto-resistive sensors, one or more magnetic field sensors, and/or one or more thermal sensors.

In an aspect, the at least one sensor component includes an acoustic scanning device capable of using focused sound to image the at least one type of microbe captured on the microbe-capture region. See, e.g., Hildebrand et al. (1981) Proc. Natl. Acad. Sci., USA. 78:1656-1660, which is incorporated herein by reference.

In an aspect, the at least one sensor component includes at least one of optical scanning, light scattering, electrical impedance, infrared spectroscopy, acoustic imaging, thermal imaging, photothermal imaging, or visible light absorption or refraction. See, e.g., Doornbos et al. (1993) Cytometry 14:589-594; Gao et al. (2003) Proceedings of the 25th Annual International Conference of the IEEE EMBS, Cancun, Mexico, Sep. 17-21, 2003; Oberreuter et al. (2002) Int. J. Syst. Evol. Microbiol. 52:91-100; Baddour et al. (2002) Ultrasonics Symposium IEEE 2:1639-1644; Zharov et al. (2006) J. Cell. Biochem. 97:916-932; Zharov et al. (2006) J. Biomed. Opt. 11:054034-1-4; Koenig et al. (1994) J. Fluoresc. 4:17-40; which are each incorporated herein by reference. In an aspect, the at least one sensor component can include a scanning laser beam and a charge-coupled device camera to acquire light scatter-image signatures. See, e.g., Huff et al. (2012) Microbial Biotechnology 5:607-620, which is incorporated herein by reference.

In an aspect, the at least one sensor component detects one or more infrared spectral properties of the at least one type of microbe on the microbe-capture region. In general, cells including microbes contain various chemical components with characteristic infrared spectra, including proteins, nucleic acids, carbohydrates and lipids. The spectra are created when a molecule converts infrared radiation into molecular vibrations. These vibrations create bands in a spectrum that occur at specific wavelengths. Differences in the chemical composition of a microbe can be distinguished by changes in spectra. For example, Fourier Transfer Infrared (FTIR) Spectroscopy can be used to distinguish Streptococcus from a virus using a spectral range of wavenumbers from 4000 to 800 cm1 (U.S. Pat. No. 6,379,920, which is incorporated herein by reference). Alternatively, FTIR data may be obtained at various frequency ranges, such as for example, 3000-2800 cm⁻¹, 1800-1500 cm⁻¹, 1500-1200 cm⁻¹, and 1200-900 cm⁻¹, and 900-700 cm⁻¹ and spectra obtained in these various ranges compared with known spectra of various bacteria. See, e.g., Oberreuter et al. (2002) Int. J. Syst. Evol. Microbiol. 52:91-100 and Helm et al. (1991) J. General Microbiology 137:69-79, which are incorporated herein by reference.

In an aspect, the at least one sensor component can include a thermal sensor, e.g., an infrared sensor. For example, the at least one sensor component can include at least one infrared photosensor, e.g., an indium gallium arsenide and/or mercury cadmium telluride based photosensor.

In an aspect, the at least one sensor component can detect one or more signals indicative of a size, a morphological property, and/or a physical feature of the at least one type of microbe captured on the microbe-capture region. For example, the at least one sensor component, e.g., an image-capture device, can be configured to detect by optical or other means the shape, outline, and/or periphery of the at least one type of microbe on the microbe-capture region. The shape, outline, and/or periphery can be further used to determine a size, a morphological property, or a physical feature of the at least one type of microbe. For example, bacteria typically range in size from 0.5 to 5.0 micrometers. Common morphologies of bacteria include spherical, e.g., cocci, or rod shaped, e.g., bacilli. Additional morphologies include corkscrew, filamentous, helical, enlarged rod, and spirochete. Physical features include hypha or stock of budding or appendaged bacteria, or flagella. In contrast, fungi can be multicellular or unicellular. Multicellular fungi are composed of filamentous hyphae. Unicellular fungi include a wide variety of budding yeast. Some fungi, such as Candida, are dimorphic with yeast phases and filamentous phases. Viruses range in size from 20 to 300 nanometers. The use of contrast agents, e.g., a tungsten heavy electron dense stain, can increase contrast to aid in visualizing viruses and other microbes. In an aspect, physical features may also include intracellular shapes, outlines, and/or peripheries, e.g., of organelles and the like, associated with a type of microbe. In an aspect, the size of the microbe is correlated with its light scattering properties. See, e.g., Ulicny (1992) Gen. Physiol. Biophys. 11:133-151, which is incorporated herein by reference.

In an aspect, the at least one type of microbe can be identified based on pattern and image recognition or signal recognition analysis. Various methods have been described for image and shape analysis of cells and subcellular components of cells. See, e.g., Fei-Fei et al. (2006) IEEE Transactions on Pattern Analysis and Machine Intelligence 28:594-611; and Martin et al. (2004) IEEE Transactions on Pattern Analysis and Machine Intelligence 26:530-549, which are incorporated herein by reference.

In an aspect, the at least one sensor component includes at least one chemical sensor. In an aspect, the at least one sensor component includes at least one electrochemical sensor, sensor chips, enose, biosensor, or cantilevers. In an aspect, the at least one sensor component includes at least one chemical sensor that is a gas sensor, such as an acoustic wave, chemiresistant, or piezoelectric sensors, or an electronic nose. One or more sensors are optionally small in size, for example a sensor or array that is a chemical sensor (see, e.g., Snow (2005) Science 307:1942-1945, which is incorporated herein by reference), a gas sensor (see, e.g., Hagleitner, et al. (2001) Nature 414:293-296, which is incorporated herein by reference), an electronic nose, and/or a nuclear magnetic resonance imager (see, e.g., Yusa (2005), Nature 434:1001-1005, which is incorporated herein by reference).

In an aspect, the at least one sensor component includes a sensor component capable of micro electrical impedance spectroscopy using MEMS and/or Lab-on-a-chip technology (see, e.g., Sun et al. (2007) Meas. Sci. Technol. 18:2859-2868; Mohanty et al., Microtechnologies in Medicine and Biology 485-488, which are incorporated herein by reference).

With reference to FIG. 10, analyzer 130 of kit 100 further includes user interface 150. User interface 150 is operably coupled to computing component 160 and includes one or more input components and/or output components for use by a user to interface with analyzer 130 of kit 100. As shown in block 1060 of FIG. 10, user interface 150 can include at least one of a display, touchscreen, keyboard, microphone, speaker, mouse, joystick, buttons, switches, or printer. The one or more input components can be used to enter information into the analyzer, e.g., user information, temporal or spatial information, operating instructions, or treatment regimen, and may be integrated into the analyzer or may be one or more peripheral devices operably connected through a wired or wireless connection to the analyzer. Non-limiting examples of input components include a graphical user interface, a display, a keyboard, a keypad, a touch-screen, a microphone, a stylus pen, a switch, a dial, or the like. In some embodiments, the user interface is user driven. For example, the user inputs data or operating conditions into the analyzer using the user interface, e.g., a touch-screen. In some embodiments, the user interface, e.g., a switch, is circuitry driven. For example, an on/off switch may be toggled based on proximity of a portion of the microbe sampling unit or the user to the analyzer.

The user interface includes one or more output components over which processed information is viewed as output results and may be integrated into the analyzer or may be one or more peripheral devices operably connected through a wired or wireless connection to the analyzer. For example, the user interface may be used to report to a user a microbe profile including a spatial distribution and/or an identity of one or more types of microbes on a body surface, e.g., a skin surface, of the user and/or the recommended at least one of the plurality of treatment agents. For example, the user interface may be used to recommend to the user at least one of the plurality of treatment agents included in the kit. Non-limiting examples of output components include but are not limited to television screens, computer monitors, liquid crystal displays, audio speakers, audio headphones, and printers.

Analyzer 130 further includes computing component 160. Computing component includes a processor and is operably coupled to user interface 150 and at least one sensor component 140. The computing component further includes circuitry 170 configured to receive the sensor output from the at least one sensor component, the sensor output including information associated with at least one property of the detected one or more signals emitted or reflected from the microbe-capture region of the microbe sampling unit; circuitry configured to compare the at least one property of the detected one or more signals emitted or reflected from the microbe-capture region of the microbe sampling unit with a reference dataset of signal properties; circuitry configured to generate a microbe profile of the user based on the comparison with the reference dataset of signal properties; circuitry configured to compare the microbe profile of the user with at least one reference microbe profile; and circuitry configured to recommend to the user at least one of the plurality of treatment agents based on the comparison with the at least reference microbe profile.

The computing component further includes circuitry configured to execute one or more instructions for operating the components of the analyzer, e.g., the at least one sensor component and the user interface. The computing component includes circuitry configured to execute one or more instructions for operating any or all other components incorporated into the analyzer, e.g., a transmission unit, a feeding mechanism, at least one reservoir, or a motor. The computing component includes circuitry configured to execute one or more instructions for receiving the sensor output from the at least one sensor component, the sensor output including information associated with at least one property of the detected one or more signals emitted or reflected from the microbe-capture region of the microbe sampling unit; one or more instructions for comparing the at least one property of the detected one or more signals emitted or reflected from the microbe-capture region of the microbe sampling unit with a reference dataset of signal properties; one or more instructions for generating a microbe profile of the user based on the comparison with the reference dataset of signal properties; one or more instructions for generating the microbe profile of the user with at least one reference microbe profile; and one or more instructions for recommending to the user at least one of the plurality of treatment agents based on the comparison with the at least reference microbe profile.

In an aspect, the computing component includes a processor, e.g., a central processing unit, for controlling one or more functions of the analyzer. The computing component further includes a system memory and a system bus that couples various system components including the system memory to the processor. The processor can include a microprocessor, a processing unit, a central processing unit (CPU), a digital signal processor (DSP), an application-specific integrated circuit (ASIC), a field programmable gate entry (FPGA), or the like, or any combinations thereof, and can include discrete digital or analog circuit elements or electronics, or combinations thereof. In an aspect, the computing component includes one or more ASICs having a plurality of pre-defined logic components. In an aspect, the computing component includes one or more FPGA having a plurality of programmable logic commands.

In an aspect, the computing component is operably coupled to one or more input/output components, e.g., one or more user interface components. In an aspect, the one or more input/output components are connected to the processor of the computing component through one or more user input interfaces that are coupled to the system bus, but may be connected by other interfaces and bus structures, such as a parallel port, game port, or a universal serial bus (USB). For example, external input components or output components may be connected to the processor through a USB port. The computing component may further include or be capable of connecting to a flash card memory. The computing component may further include or be capable of connecting with a network through a network port and network interface, and through wireless port and corresponding wireless interface may be provided to facilitate communication with other peripheral devices, for example, a smart phone, a computer, a display monitor, and/or a printer.

In an aspect, image-based applications such as viewers and/or toolkits (e.g., Insight Segmentation and Registration Toolkit (ITK)), are incorporated for further intake of information. In an aspect, CAD implementations, image segmentation, or other image analysis algorithms may allow processing of images received from the at least one sensor component.

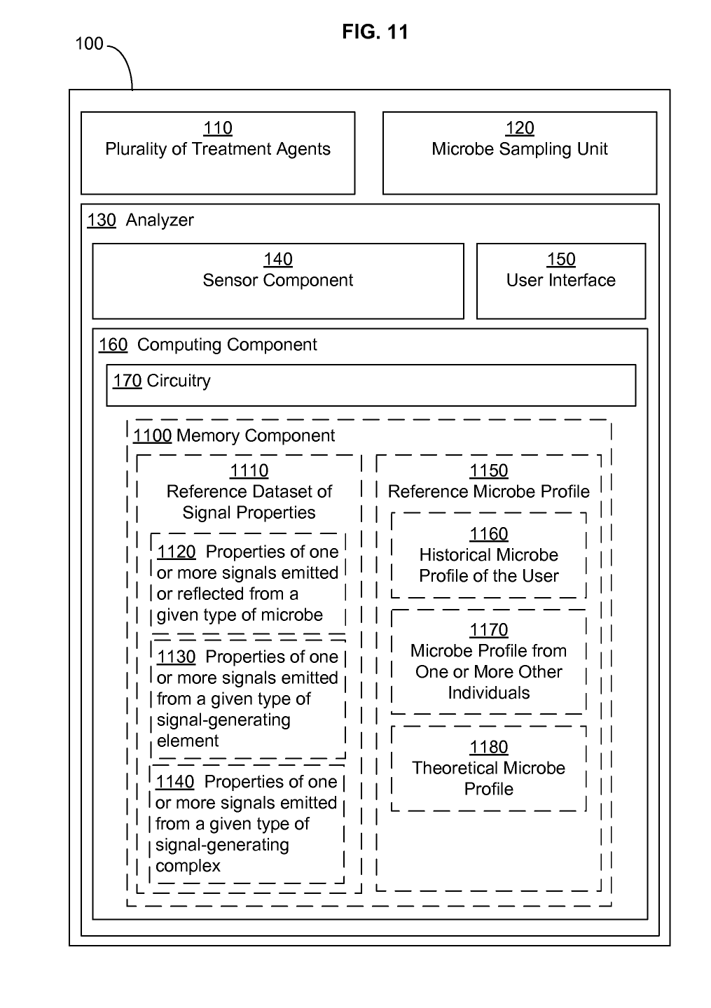

With reference to FIG. 11, computing component 160 of analyzer 130 can include memory component 1100. Memory component 1100 can include memory chips, e.g., ROM or flash memory chips, for providing storage of operating systems, look-up tables, references datasets, and algorithms for comparing input data or information with reference data or information. The memory component of the computing component may include read-only memory (ROM) and random access memory (RAM). A number of program modules may be stored in the ROM or RAM, including an operating system, one or more application programs, other program modules and program data.

The computing component includes computer-readable media products and may include any media that can be accessed by the computing component including both volatile and nonvolatile media, removable and non-removable media. By way of example, and not of limitation, computer-readable media may include non-transitory signal-bearing media. Non-limiting examples of non-transitory signal-bearing media include a recordable type medium such as magnetic tape, a hard disk drive, digital tape, computer memory, or the like, as well as transmission type medium such as a digital and/or analog communication medium (e.g., fiber optic cable, waveguide, wired communications link, wireless communication link). Further non-limiting examples of signal-bearing media include, but are not limited to, flash memory, magnetic tape, MINIDISC, non-volatile memory card, EEPROM, optical disk, optical storage, RAM, ROM, system memory, web server, cloud, or the like. By way of example, and not of limitation, computer-readable media may include computer storage media, e.g., magnetic tape, magnetic disk storage, optical disk storage, memory cards, flash memory cards, electrically erasable programmable read-only memory (EEPROM), solid state RAM, and solid state ROM or any other medium which can be used to store the desired information and which can be accessed by the computing component. By way of further example, and not of limitation, computer-readable media may include a communication media, e.g., wired media, such as a wired network and a direct-wired connection, and wireless media such as acoustic, RF, optical, and infrared media.

In an aspect, the reference dataset of signal properties is included in a memory component of the computing component of the analyzer. As shown in FIG. 11, memory component 1100 can include reference dataset of signal properties 1110. Reference dataset of signal properties 1100 includes one or more reference signal properties for comparison with the detected one or more signals emitted or reflected from the microbe-capture region of the microbe sampling unit. In an aspect, the at least one property of the detected one or more signal emitted or reflected from the microbe-capture region of the microbe sampling unit includes at least one of an optical property, a fluorescent property, a magnetic property, an electrical property, an electromagnetic property, an acoustic property, a radioactive property, or a radiofrequency property. In an aspect, reference dataset of signal properties 1100 includes properties 1120 of one or more signals emitted or reflected from a given type of microbe. For example, the reference dataset can include optical or autofluorescence properties of a given type of microbe. In an aspect, reference dataset of signal properties 1100 includes properties 1130 of one or more signals emitted from a given type of signal-generating element. For example, the reference dataset can include at least one of optical, fluorescence, electromagnetic, magnetic, electrical, acoustic, radioactive, or radiofrequency properties of a given type of signal-generating element. In an aspect, reference dataset of signal properties 1100 includes properties 1140 of one or more signals emitted from a given type of signal-generating complex. For example, the reference dataset can include at least one of optical, fluorescence, electromagnetic, magnetic, electrical, acoustic, radioactive, or radiofrequency properties of a given type of signal-generating complex.

In an aspect, the at least one reference microbe profile is included in a memory component of the computing component of the analyzer. With reference to FIG. 11, in an aspect, memory component 1100 of computing component 160 includes at least one reference microbe profile 1150. In an aspect, at least one reference microbe profile 1150 includes at least one historical microbe profile of the user 1160. In an aspect, the at least one historical microbe profile of the user includes at least one microbe profile generated at a previous point in time, e.g., at a younger age. The at least one historical microbe profile of the user can include at least one microbe profile generated one or more days, one or more weeks, and/or one or more years previous to a current point in time. The at least one historical microbe profile of the user can include at least one microbe profile generated at a point in time before onset of a condition and/or before onset of a treatment.

In an aspect, at least one reference microbe profile 1150 includes at least one microbe profile from one or more other individuals 1170. For example, the at least one microbe profile from one or more other individuals can include a microbe profile averaged or normalized from a number of individuals matched to the user, e.g., matched in age, gender, ethnicity, geographical location, medical condition, or co-morbidities. In an aspect, the at least one microbe profile from one or more other individuals can include a microbe profile averaged or normalized from one or more idealized individuals based on the user's preferences. For example, the at least one reference microbe profile can include a microbe profile of one or more individuals of an age, gender, ethnicity, skin characteristics, geographical location, medical history, or co-morbidities that the user wishes to emulate. For example, the at least one microbe profile from one or more other individuals can include a microbe profile of an admired individual, e.g., a celebrity.

In an aspect, at least one reference microbe profile 1150 includes at least one theoretical microbe profile 1180. In an aspect, the at least one theoretical microbe profile includes a microbe profile compiled from a number of microbe profiles to form a standardized microbe profile. In an aspect, the at least one theoretical microbe profile includes a microbe profile generated by a computer based from a number of microbe profile to form a standardized microbe profile. In an aspect, the at least one theoretical microbe profile includes an optimized or ideal microbe profile, e.g., a microbe profile including a generally recognized balance of beneficial commensal microbes. In an aspect, the at least one theoretical microbe profile includes a marginal or bad microbe profile. For example, the theoretical microbe profile might include a disease- or condition-associated microbe profile, e.g., acne, psoriasis, Crohn's disease, diabetes, or other disease or condition.

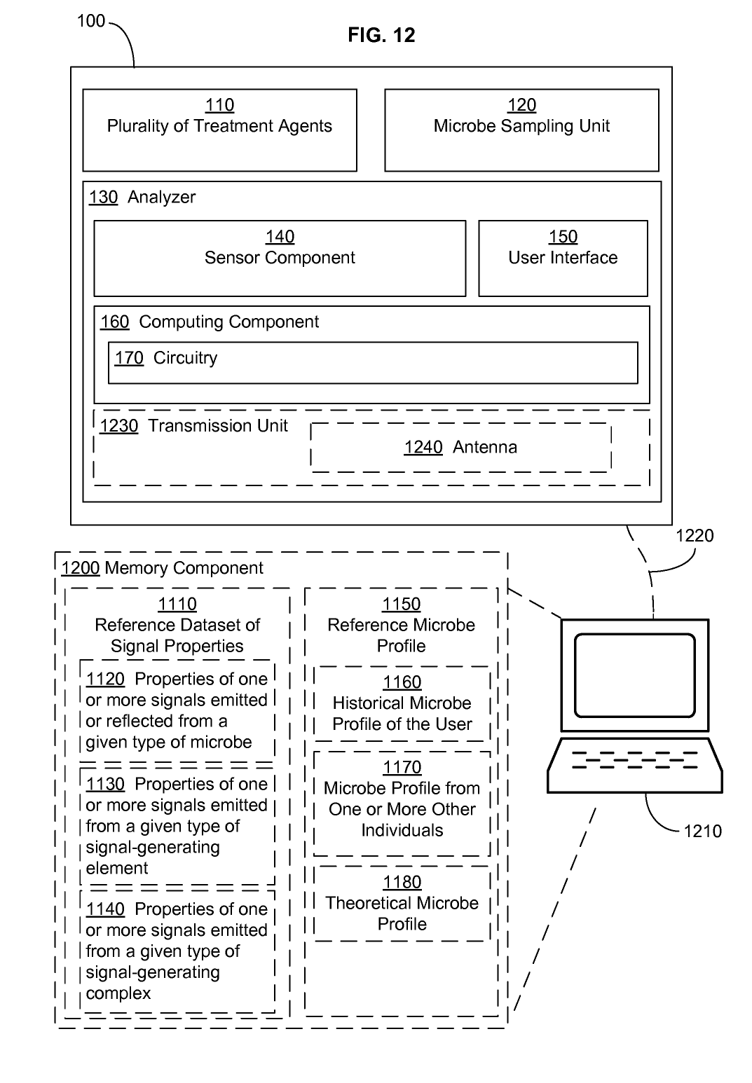

In an aspect, the reference dataset of signal properties and the at least one reference microbe profile are incorporated into a memory component of the computing component of the analyzer. In an aspect, as shown in FIG. 12, the reference dataset of signal properties 1110 is accessed from a remote computing device 1210 through a communication link 1220. In an aspect, the at least one reference microbe profile 1150 is accessed from a remote computing device 1210 through a communication link 1220. In an aspect, reference dataset of signal properties 1110, including properties 1120 of one or more signals emitted or reflected from a given type of microbe, properties 1130 of one or more signals emitted from a given type of signal-generating element, and/or properties 1140 of one or more signals emitted from a given type of signal-generating complex, is stored in memory component 1200 of remote computing device 1210. In an aspect, at least one reference microbe profile 1150, including at least one historical microbe profile of the user 1160, at least one microbe profile from one or more other individuals 1170, and/or at least one theoretical microbe profile 1180, is stored in memory component 1200 of remote computing device 1210. In an aspect, the remote computing device is associated with at least one of a medical practice, a laboratory, a supplier, a retailer, a manufacturer, or other like entity. In an aspect, the remote computing device is associated with a remote server, a cloud-based server, a web-based server. In an aspect, the communication link includes a wired communication link, e.g., a cable communication. In an aspect, the communication link includes a wireless communication link.

In an aspect, analyzer 130 further includes transmission unit 1230 including antenna 1240. In an aspect, transmission unit 1230 is operably coupled to computing component 160. A “transmission unit,” as used herein, can be one or more of a variety of units that are configured to send and/or receive signals, such as signals carried as electromagnetic waves. A transmission unit generally includes at least one antenna and associated circuitry. A transmission unit can include a transmitter and a receiver. A transmission unit can include volatile or non-volatile memory. A transmission unit can include a processor and/or be operably connected to a processor. A transmission unit can be operably connected to an energy source, such as a battery. A transmission unit can include an energy harvesting unit, such as a unit configured to obtain energy from electromagnetic waves. A transmission unit can include a transponder utilizing electromagnetic waves, for example as described in “Fundamental Operating Principles,” in Chapter 3 of the RFID Handbook: Fundamentals and Applications in Contactless Smart Cards and Identification, Klaus Finkenzeller, John Wiley & Sons, (2003), which is incorporated herein by reference. A transmission unit can include an oscillator and encoder configured to generate a programmable pulse position-modulated signal in the radio frequency range (see, e.g., U.S. Pat. No. 4,384,288, which is incorporated herein by reference). A transmission unit can include a radio frequency identification device (RFID), which can be a passive RFID device, a semi-passive RFID device, or an active RFID device, depending on the embodiment (see, e.g., Chawla & Ha, “An Overview of Passive RFID,” IEEE Applications and Practice, 11-17 (September 2007), which is incorporated herein by reference). A transmission unit including an RFID device can be configured to transmit signals in the UHF standard range. A transmission unit can include a battery-assisted passive RFID device, such as sold by Alien Technology®, Morgan Hill, Calif. A transmission unit can include an optical transmission unit. A transmission unit can include a hybrid backscatter system configured to function in an RFID, IEEE 802.11x standard and Bluetooth system (see, e.g., U.S. Pat. No. 7,215,976, which is incorporated herein by reference). A transmission unit can include a near field communication (NFC) device. A transmission unit can include a Wireless Identification and Sensing Platform (WISP) device.

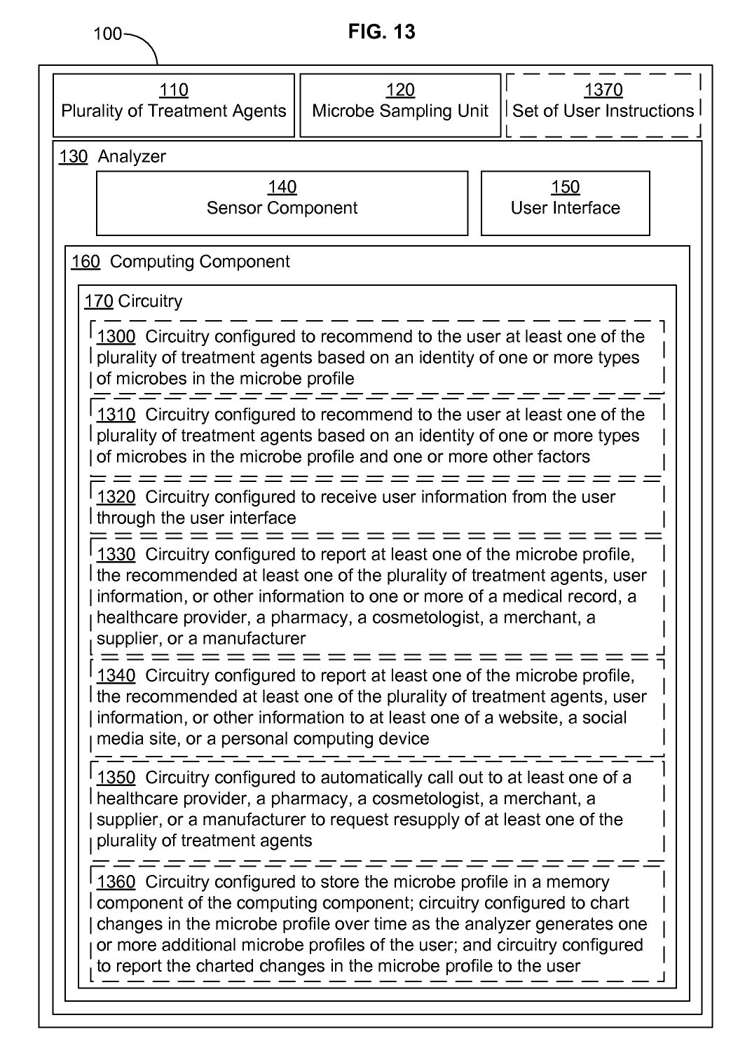

FIG. 13 shows further aspects of a kit such as shown in FIG. 1. Kit 100 includes a plurality of treatment agents 110, microbe sampling unit 120, and analyzer 130. Analyzer 130 further includes at least one sensor component 140, user interface 150, and computing component 160. Computing component 160 includes circuitry 170. In an aspect, circuitry 170 includes circuitry configured to recommend to the user at least one of the plurality of treatment agents 110. In an aspect, computing component 160 of analyzer 130 includes circuitry 1300 configured to recommend to the user at least one of the plurality of treatment agents based on an identity of one or more types of microbes in the microbe profile. For example, the computing component can include a dataset, database, and/or look-up table including information matching specific microbes with specific treatment options. For example, the computing component can include a dataset, database, and/or look-up table including information matching specific microbe with one or more probiotic agents, prebiotic agents, and/or antimicrobial agents. In an aspect, computing component 160 of analyzer 130 includes circuitry 1310 configured to recommend to the user at least one of the plurality of treatment agents based on an identity of one or more types of microbe in the microbe profile and one or more factors. In an aspect, the one or more other factors includes one or more of age, gender, ethnicity, skin characteristic, geographical location, medical history, co-morbidities, or user preference. For example, the age of a user, e.g., young versus old, may dictate the types of microbes present on a given body surface. See, e.g., Yatsunenko et al. (2012) Nature 486:222-227 and Oh et al. (2012) Genome Medicine 4:77, which are incorporated herein by reference. For example, the ethnicity and/or geographical location of the user may dictate the types of microbes are present on a given body surface. See, e.g., Mason et al. (2013) PLoSONE 8(10):e77287 and Shetty et al. (2013) Microbiome 1:24, which are incorporated herein by reference. For example, a skin characteristic such as whether the skin surface is sebaceous, moist, or dry may dictate the types of microbes present on said skin surface. See, e.g., Orrice et al. (2009) Science 324:1190-1192. In an aspect, the skin characteristics dictate which vehicle (cream, gel, lotion, or solution) is appropriate for a skin type. For example, creams may be appropriate for users with sensitive or dry skin, but too “oily” for users with oily skin; user's with oily skin may be more comfortable with gels that have drying effects, but may prevent cosmetics from adhering; lotions may be used for all skin types, but may also have burning or drying effects; and solutions, e.g., solutions of antimicrobials, are often dissolved in alcohol, which may dry the skin. For example, the microbe profile might dictate which probiotic agents, prebiotic agents, and/or antimicrobial agents are recommended, while the one or more other factors, e.g., age, gender, skin characteristics, or medical history, might dictate which therapeutic agents, moisturizers, sunscreens, or cosmetic agents are recommended.

In an aspect, the one or more other factors include at least one user preference. For example, the user preference can include a preferred reference microbe profile for comparison with the microbe profile, e.g., a preferred historical microbe profile of the user or a preferred microbe profile of one or more other individuals, e.g., an idealized microbe profile or a celebrity microbe profile. In an aspect, circuitry 170 includes circuitry 1320 configured to receive user information from the user through user interface 150. In an aspect, the user information includes at least one of age, gender, ethnicity, skin characteristic, geographical location, medical history, co-morbidities or user preference.

In an aspect, computing component 160 of analyzer 130 includes circuitry 1330 configured to report at least one of the microbe profile, the recommended at least one of the plurality of treatment agents, user information, or other information to one or more of a medical record, a healthcare provider, a pharmacy, a cosmetologist, a merchant, a supplier, or a manufacturer. In an aspect, computing component 160 of analyzer 130 includes circuitry 1340 configured to report at least one of the microbe profile, the recommended at least one of the plurality of treatment agents, user information, or other information to at least one of a website, a social media site, or a personal computing device. In an aspect, computing component 160 of analyzer 130 includes circuitry 1350 configured to automatically call out to at least one of a healthcare provider, a pharmacy, a cosmetologist, a merchant, a supplier, or a manufacturer to request resupply of at least one of the plurality of treatment agents. In an aspect, computing component 160 of analyzer 130 includes circuitry 1360 configured to store the microbe profile in a memory component of the computing component; circuitry configured to chart changes in the microbe profile over time as the analyzer generates one or more additional microbe profiles of the user; and circuitry configured to report the charted changes in the microbe profile to the user.

In an aspect, kit 100 further includes a set of user instructions 1370 for using the kit. In an aspect, the set of user instructions 1370 for using the kit includes at least one of one or more instructions for sampling the body surface of the user with the microbe sampling unit, one or more instructions for using the analyzer, one or more instructions for interpreting the microbe profile, one or more instructions for applying the recommended at least one of the plurality of treatment agents to the body surface, or one or more instructions for accessing a website. In an aspect, the set of user instructions is provided to the user as a sheet of paper with written user instructions. In an aspect, the set of user instructions is on non-transitory machine readable media. In an aspect, the set of user instructions on non-transitory machine readable media are provided to the user on the user interface, e.g., a display, a printout, or an audio recording.

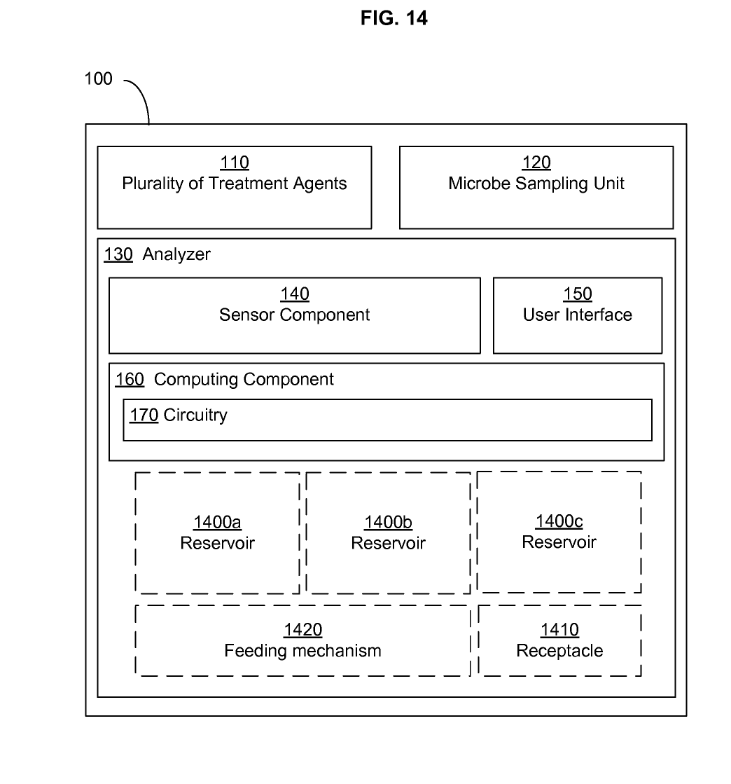

With reference to FIG. 14, shown are further embodiments of a kit. In an aspect, analyzer 130 of kit 100 includes one or more reservoirs 1400a, 1400b, and 1400c. The one or more reservoirs are configured to store one or more processing reagents for processing microbe sampling unit 120 prior to analysis with at least one sensor component 140 of analyzer 130. In an aspect, at least one of one or more reservoirs 1400a, 1400b, and 1400c includes a plurality of at least one type of signal-generating element. Non-limiting examples of signal-generating elements have been described above herein. In an aspect at least one of one or more reservoirs 1400a, 1400b, and 1400c includes at least one of a buffer, a detergent solution, a reagent solution, or a wash solution. For example, a microbe sampling unit may be exposed to a fluorescently labeled antibody in a first reservoir and excess fluorescently labeled antibody removed from the microbe sampling unit with at least one of a buffer, detergent solution, or wash solution in at least one second reservoir. For example, a microbe sampling unit may go through a series of staining and washing steps by moving through one reservoir to another. In an aspect, the buffer includes any of a number of biocompatible buffering agents, non-limiting examples of which include saline sodium citrate, PBS (phosphate buffered saline), Tris(tris(hydroxymethyl)methylamine), Tricine (N-tris(hydroxymethyl)methylglycine), HEPES (4-2-hydroxyethyl-1-piperazineethanesulfonic acid) MOPS (3-(N-morpholino)propanesulfonic acid) and PIPES (piperazine-N,N′-bis(2-ethanesulfonic acid)). In an aspect, the detergent includes any of a number of detergents used for biological applications, non-limiting examples of which include ionic detergents, e.g., sodium dodecyl sulfate (SDS) or cetyl methyl ammonium bromide (CTAB), non-ionic detergents, e.g., TRITON-X-100, or Zwitterionic detergents, e.g., CHAPS. In an aspect, the reagents solution can include any of a number of reagents necessary for a processing the microbe sampling unit prior to analysis. For example, the reagent solution may include the reagents necessary for immunochemical processing of the microbe sampling unit. For example, the reagent solution may include reagents necessary for RNA or DNA hybridization processing of the microbe sampling unit. For example, the reagent solution may include reagents necessary of DNA/RNA amplification processing of the microbe sampling unit. In an aspect, the wash solution may include water, saline, alcohol, a buffer, e.g., PBS, a detergent solution, a stringency salt solution, or a combination thereof. For example, a wash solution for immunochemical analysis might include a 1% TRITON-X-100 solution in PBS.

In an aspect, analyzer 130 includes a receptacle 1410 sized to fit at least a portion of the microbe sampling unit. In an aspect, the analyzer includes a receptacle sized to fit at least a portion of a mask, mouthpiece, strip, swab, brush, or razor. In an aspect, the analyzer includes a surface for scanning at least one surface of the microbe sampling unit with the at least one sensor component. For example, the surface can include a scanning surface with a closable flap such as in a typical flatbed scanning device. For example, the analyzer may include a door which opens revealing a receptacle for inserting at least a portion of the microbe sampling unit. For example, the analyzer may include a slit in a side of the analyzer through which at least a portion of the microbe sampling unit may be inserted.

In an aspect, analyzer 130 includes a feeding mechanism 1420 for feeding microbe sampling unit 120 into contact with at least the one or more reservoirs for pre-analysis processing and/or the at least one sensor component for analysis. In an aspect, feeding mechanism 1420 is configured to convey, e.g., pull and/or push, at least a portion of the microbe sampling unit into the analyzer for analysis. In an aspect, feeding mechanism 1420 is configured to pull and/or push at least a portion of the microbe sampling unit into contact with at least one sensor component 140. In an aspect, feeding mechanism 1420 is configured to pull at least a portion of the microbe sampling unit into contact with the contents of one or more reservoirs 1400a, 1400b, and/or 1400c for treatment with one or more reagents, e.g., signal-generating elements, prior to analysis with the at least one sensor component.

### Kiosk

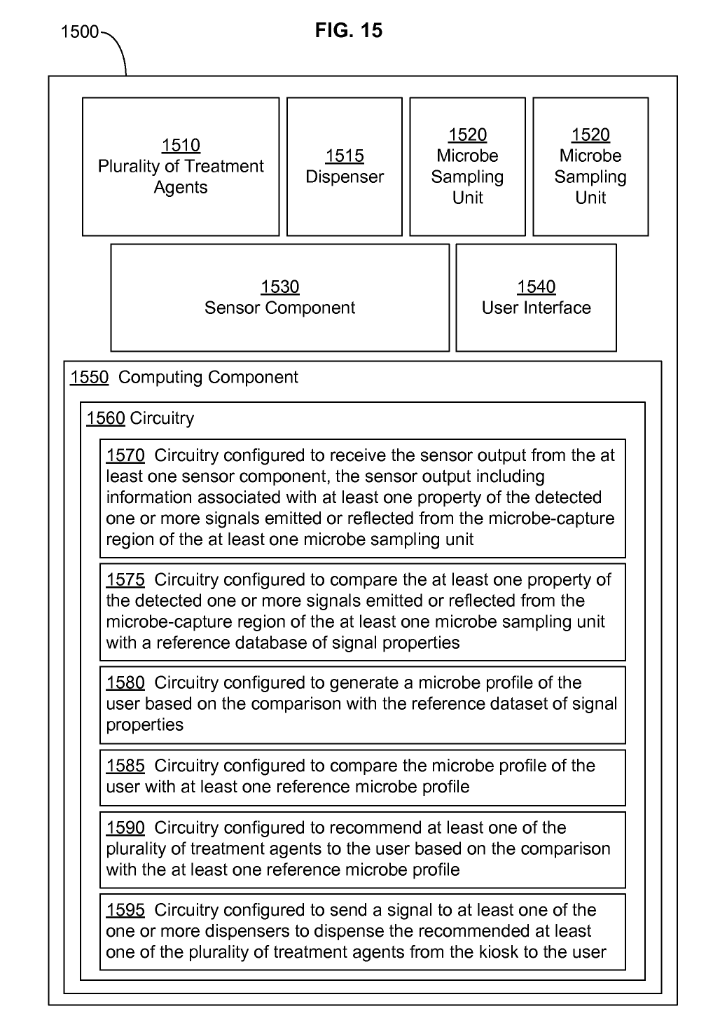

A kiosk is described for sampling microbiota of a user, analyzing the sampled microbiota, generating a microbe profile, recommending use at least one of a plurality of treatment agents, and dispensing the at least one of the plurality of treatment agents. With reference to FIG. 15, kiosk 1500 includes plurality of treatment agents 1510; one or more dispensers 1515 to dispense at least one of plurality of treatment agents 1510; at least one microbe sampling unit 1520 including at least one surface with a microbe-capture region, the microbe capture region configured to capture one or more types of microbes from a body surface of a user; user interface 1540; at least one sensor component 1530 including circuitry configured to detect one or more signals emitted or reflected from the microbe-capture region of at least one microbe sampling unit 1520 and to transform the detected one or more signals into a signal output; and computing component 1550. Computing component 1550 includes a processor and is operably coupled to one or more dispensers 1515, at least one sensor component 1530, and user interface 1540. Computing component 1550 includes circuitry 1560. Circuitry 1560 includes circuitry 1570 configured to receive the sensor output from at least one sensor component 1530, the sensor output including information associated with at least one property of the detected one or more signals emitted or reflected from the microbe-capture region of at least one microbe sampling unit 1520. Circuitry 1560 includes circuitry 1575 configured to compare the at least one property of the detected one or more signals emitted or reflected from the microbe-capture region of at least one microbe sampling unit 1520 with a reference dataset of signal properties. Circuitry 1560 includes circuitry 1580 configured to generate a microbe profile of the user based on the comparison with the reference dataset of signal properties. Circuitry 1560 includes circuitry 1585 configured to compare the microbe profile of the user with at least one reference microbe profile. Circuitry 1560 includes circuitry 1590 configured to recommend at least one of the plurality of treatment agents 1510 to the user based on the comparison with the at least one reference microbe profile. Circuitry 1560 includes circuitry 1595 configured to send a signal to at least one of the one or more dispensers 1515 to dispense the recommended at least one of the plurality of treatment agents 1510 from kiosk 1500 to the user.

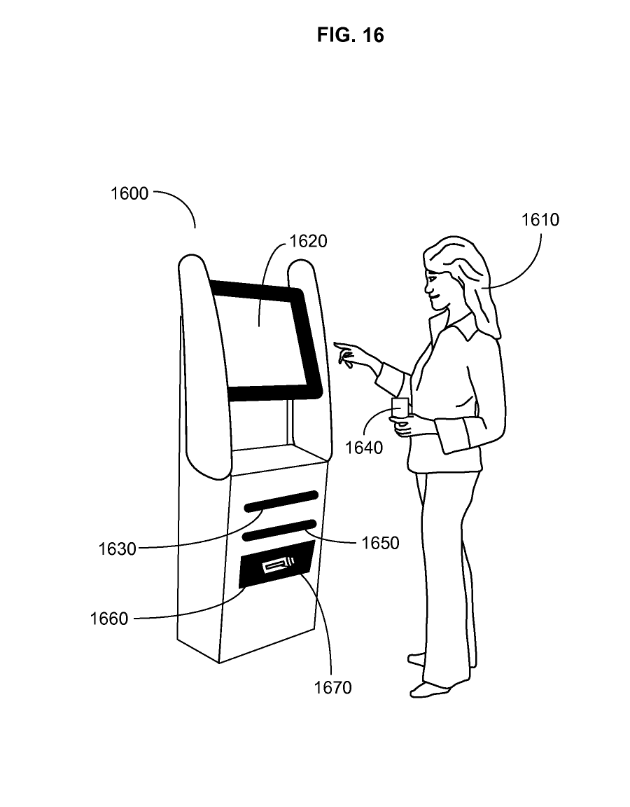

FIG. 16 illustrates a non-limiting example of kiosk 1600 with user 1610. Kiosk 1600 includes a user interface, e.g., a touchscreen display 1620 for use by user 1610 to interact with kiosk 1600. For example, the kiosk can include a set of user instructions displayed on the user interface, the set of user instructions including instructions for using the kiosk, a microbe sampling unit, and/or one or more of the plurality of treatment agents. Kiosk 1600 further includes microbe sampling unit dispenser component 1630 configured to dispense to user 1610 at least one microbe sampling unit 1640, and receiving component 1650 configured to receive from user 1610 a used at least one microbe sampling unit 1640 for analysis. Kiosk 1600 includes at least one sensor component for detecting one or more signals emitted or reflected from the microbe-capture region of the received microbe sampling unit. Kiosk 1600 further includes a computing component and circuitry configured to generate a microbe profile of the user and recommend at least one of a plurality of treatment agents included in kiosk 1600. Kiosk 1600 includes one or more dispenser 1660 for dispensing at least one of a plurality of treatment agents 1670 to the user based on the recommendation formulated by the computing component.

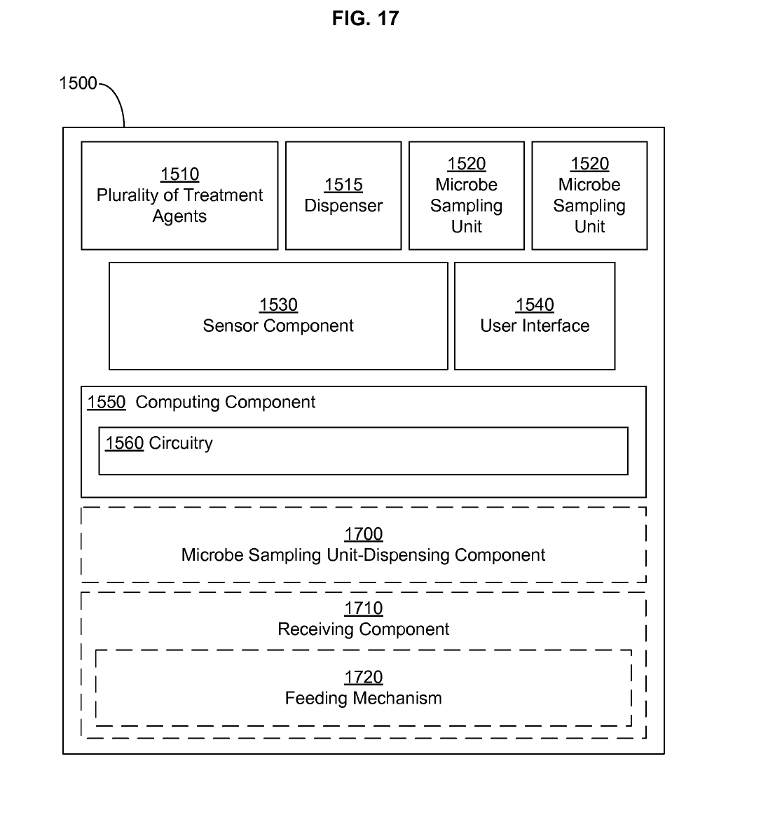

FIG. 17 shows further aspects of a kiosk. Kiosk 1500 includes one or more dispensers 1515 to dispense at least one of the plurality of treatment agents. In an aspect, the one or more treatment agents are pre-packaged in a container for dispensing. In an aspect, the one or more dispensers include a mechanical dispensing coil which when activated drops the selected treatment agents into a collection bin. In an aspect, the one or more dispensers include a spiraled dispenser driven by a motor that pushes the selected treatment agents into a collection bin. In an aspect, the one or more dispensers include one or more of a door or drawer that when unlocked allows access to the selected treatment agents. In an aspect, the one or more dispensers include a claw crane that actively picks up the selected treatment agents and drops or places said selected treatment agents into the collection bin.

In an aspect, the one or more treatment agents are stored in bulk in kiosk 1500. In an aspect, one or more dispensers 1515 dispense aliquots, doses, or portions of the one or more treatment agents into a container, e.g., a glass or plastic bottle or vial. In an aspect, one or more dispensers 1515 are configured to dispense a liquid, gel, solid, or powder form of the one or more treatment agents.

In an aspect, kiosk 1500 includes microbe sampling unit-dispensing component 1700 operably coupled to computing component 1550, microbe sampling unit dispensing component 1700 configured to dispense the at least one microbe sampling unit to the user. In an aspect, the microbe sampling unit dispensing component includes at least one of a screw, coil, or claw system for dispensing the at least one microbe sampling unit into a collection bin. In an aspect, the microbe sampling unit dispensing component includes a conveyor system for moving the microbe sampling unit out of a storage area from within the kiosk. In an aspect, the collection bin for dispensing the microbe sampling unit is the same collection bin used for dispensing the at least one of the plurality of treatment agents. In an aspect, separate collection bins are used for dispensing the one or more treatment agents and the microbe sampling unit.

In an aspect, kiosk 1500 includes at least one receiving component 1710 operably coupled to computing component 1550, at least one receiving component 1710 configured to receive the at least one microbe sampling unit from the user. In an aspect, the at least one receiving component includes a receiving bin into which the microbe sampling unit is placed or fed. In an aspect, at least one receiving component 1710 includes feeding mechanism 1720. In an aspect, the feeding mechanism includes a conveyor system, e.g., rollers and/or belts, for grabbing and moving the microbe sampling unit through an opening of the kiosk and into proximity to the at least one sensor component for analysis.

In an aspect, kiosk 1500 further includes a bill acceptor to accept payment for use of the kiosk. In an aspect, kiosk 1500 further includes a payment item acceptor, e.g., a payment card acceptor or a credit card acceptor, to accept payment for use of the kiosk. In an aspect, kiosk 1500 includes a biometric sensor to accept payment for use of the kiosk. In an aspect, kiosk 1500 includes an electronic acceptor to accept payment for use of the kiosk. In an aspect, kiosk 1500 includes an interface, for example a pin number keypad and/or a signature space, to accept payment for use of the kiosk.

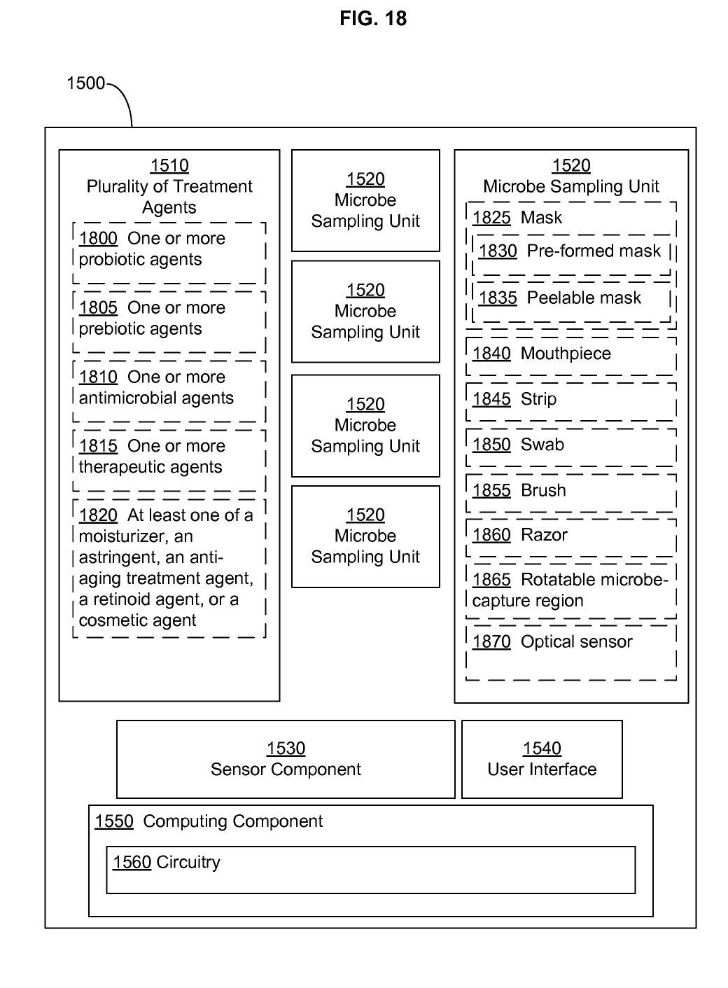

FIG. 18 shows further aspects of a kiosk. In an aspect, kiosk 1500 includes a plurality of treatment agents 1510. In an aspect, one or more of the plurality of treatment agents are contained in one or more dispensable containers. In an aspect, the kiosk includes two or more types of dispensable containers, each type of dispensable container including one or more treatment agents. In an aspect, each type of dispensable container includes a single treatment agent. In an aspect, each type of dispensable container includes a combination of treatment agents. In an aspect, the plurality of treatment agents include at least one first treatment agent in at least one first container and at least one second treatment agent in at least one second container. In an aspect, the plurality of treatment agents includes at least one third treatment agent in at least one third container. In an aspect, the at least one first container, the at least one second container, and/or the at least one third container includes a dispensable container. In an aspect, each container, e.g., each dispensable container, is labeled with a machine-readable code, e.g., an alphanumeric code or a bar code, readable by a code reader of the kiosk.

In an aspect, one or more of the plurality of treatment agents are contained in two or more large containers within the kiosk and dispensed into one or more small containers at the time of dispensing. For example, one or more treatment agents can be stored in bulk within the kiosk and dispensed through the one or more dispensers into a waiting small container. In an aspect, the plurality of treatment agents include at least one first treatment agent dispensable by at least one first dispenser and at least one second treatment agent dispensable by at least one of the at least one first dispenser or at least one second dispenser. In an aspect, the at least one first treatment agent is stored in a first large container, the at least one second treatment agent is stored in a second large container, and the at least one third agent is stored in a third large container, the at least one first, second, or third treatment agent dispensed from its respective large container into a waiting small container.

In an aspect, plurality of treatment agents 1510 includes one or more probiotic agents 1800. In an aspect, the one or more probiotic agents include at least one type of bacteria from Firmicutes, Actinobacteria, Bacteriodetes, Proteobacteria, or Cyanobacteria. In an aspect, the one or more probiotic agents comprise at least one type of bacteria from Corynebacteria, Propionibacteria, Micrococci, or Staphylococci. In an aspect, the one or more probiotic agents include at least one of a non-pathogenic strain of a pathogenic bacterium. In an aspect, the one or more probiotic agents include Staphylococcus epidermidis. In an aspect, plurality of treatment agents 1510 includes one or more prebiotic agents 1805. In an aspect, the one or more prebiotic agents include at least one of an oligosaccharide, inulin, or lactulose. In an aspect, plurality of treatment agents 1510 includes one or more antimicrobial agents 1810. In an aspect, the one or more antimicrobial agents include at least one of an antibacterial agent, an antifungal agent, or an antiviral agent. In an aspect, plurality of treatment agents 1510 includes one or more therapeutic agents 1815. In an aspect, the one or more therapeutic agents include at least one of an anti-inflammatory agent, a chemotherapeutic agent, an antiseptic, an anesthetic, or an anti-acne agent. In an aspect, plurality of treatment agents 1510 includes at least one of a moisturizer, an astringent, an anti-aging treatment agent, a retinoid agent, or a cosmetic agent as shown in block 1820. Non-limiting examples of probiotic agents, prebiotic agents, antimicrobial agents, therapeutic agents, and other treatment agents have been described above herein.

In an aspect, the plurality of treatment agents 1510 of kiosk 1500 includes two or more treatment agents to modulate one or more types of microbes on a body surface of a user. In an aspect, the plurality of treatment agents 1510 of kiosk 1500 includes two or more treatment agents to modulate one or more types of microbes on a skin surface of a user. It should be understood that this disclosure refers to skin by example only but is also intended to include other body surfaces including but not limited to a gastrointestinal surface, a mucosal surface, a vaginal surface, a nasal surface, or an oral surface of a user. In an aspect, the plurality of treatment agents includes two or more treatment agents for application to a gastrointestinal surface, mucosal surface, a vaginal surface, a nasal surface, and/or an oral surface to modulate one or more types of microbe on said body surface of the user. In an aspect, to modulate one or more types of microbes includes inhibiting the growth of one or more types of microbes. In an aspect, to modulate one or more types of microbe includes promoting the growth of one or more types of microbes. In an aspect, to modulate one or more types of microbes includes preventing the attachment of one or more types of microbes. In an aspect, to modulate one or more types of microbes includes preventing the growth of one or more types of microbes. In an aspect, to modulate one or more types of microbes includes preventing the colonization of one or more types of microbes. In an aspect, at least one of the plurality of treatment agents includes at least one agent to modulate an environment. In an aspect, at least one of the plurality of treatment agents includes at least one agent to modulate an environment, e.g., to influence one or more types of microbes. In an aspect, at least one of the plurality of treatment agents includes at least one agent to modulate an environment, e.g., the pH of a body surface, to prevent, inhibit, or promote the attachment, growth, or colonization of one or more types of microbes.

In an aspect, the plurality of treatment agents include one or more agents configured to maintain, alter, and/or improve the microbiota, e.g., the types and quantity of microorganisms, on a body surface of a user, e.g., a skin, gastrointestinal, vaginal, nasal, or oral surface of the user. In an aspect, the one or more treatment agents include one or more agents configured to treat a condition or disease on the body surface of the user. In an aspect, the one or more treatment agents include one or more agents configured to treat a condition or disease on the skin surface of the user.

With reference to FIG. 18, kiosk 1500 includes at least one microbe sampling unit 1520. In an aspect, kiosk 1500 includes a supply of microbe sampling units 1520 for dispensing to a user. In an aspect, the dispensed microbe sampling unit is used immediately by the user and analyzed. In an aspect, the dispensed microbe sampling unit is used by the user at a remote location and returned to the kiosk for analysis. In an aspect, the at least one microbe sampling unit includes mask 1825. In an aspect, mask 1825 includes pre-formed mask 1830. In an aspect, a mask 1825 includes peelable mask 1835. In an aspect, the at least one microbe sampling unit includes mouthpiece 1840. In an aspect, the at least one microbe sampling unit includes strip 1845. In an aspect, the microbe sampling unit, e.g., a mask or a strip, substantially conforms to a topography of the body surface of the user. In an aspect, the at least one microbe sampling unit is personalized for the user. In an aspect, the at least one microbe sampling unit includes swab 1850. In an aspect, the at least one microbe sampling unit includes brush 1855. In an aspect, the at least one microbe sampling unit includes razor 1860. In an aspect, the at least one microbe sampling unit includes a rotatable microbe-capture region 1865. Non-limiting examples of microbe sampling units have been described above herein.

In an aspect, at least one microbe sampling unit 1520 includes an optical sensor 1870. For example, the microbe sampling unit can include a mask with at least one optical sensor embedded in a surface of the mask. In an aspect, the at least one microbe sampling unit including an optical sensor is tethered to the kiosk and operably coupled to the computing component. For example, a mask including at least one optical sensor embedded in a surface of the mask can be tethered to the kiosk through a wired communication link, e.g., through a cable. In an aspect, the at least one microbe sampling unit including an optical sensor is reusable. In an aspect, the at least one optical sensor of the microbe sampling unit includes at least one photodetector. In an aspect, the at least one optical sensor of the microbe sampling unit includes at least one charged-coupled device, photodiode, quantum dot photoconductors or photodiodes, complementary metal-oxide-semiconductor (CMOS) device, active-pixel sensors, reverse-biased light emitting diode, or any other sensor type capable of detecting an optical electromagnetic signal. In an aspect, the microbe sampling unit includes an electromagnetic energy emitter, e.g., at least one light emitting diode, as well as at least one optical sensor configured to detect one or more signals emitted or reflected from a body surface of the individual. In an aspect, an electromagnetic energy emitter of the microbe sampling unit elicits a light-emitting response from the body surface of the individual. In an aspect, the electromagnetic energy emitter of the microbe sampling unit elicits a fluorescence response from at least one signal-generating element, e.g., a fluorescence signal-generating agent, associated with microbes on the body surface. For example, the body surface of the individual can be treated with a fluorescence signal-generating element, e.g., a fluorescing antibody, that interacts with one or more types of microbes, the interaction detected with the at least one optical sensor of the microbe sampling unit. For example, the microbe sampling unit can include a light source of appropriate wavelength to elicit a fluorescence response from one or more types of fluorescently labeled microbes. In an aspect, an electromagnetic energy emitter of the microbe sampling unit elicits an autofluorescence response from one or more types of microbes on the body surface of the individual. For example, the microbe sampling unit can include a light source of appropriate wavelength to elicit an autofluorescence response from one or more types of microbes on the skin surface of a user. In an aspect, the response elicited by the electromagnetic energy emitter, e.g., one or more signals emitted or reflected the on the body surface, is detected by the at least one optical sensor. In an aspect, the microbe sampling unit including the at least one optical sensor is operably coupled to the computing component of the kiosk, e.g., through a wired or wireless communication link, the computing component of the kiosk including circuitry configured to receive sensor output from the at least one optical sensor of the microbe sampling unit, the sensor output including at least one property of the one or more signals emitted or reflected from the body surface of the individual, compare the at least one property of the detected one or more signals emitted or reflected from the body surface of the individual with a reference dataset of signal properties, generate a microbe profile of the user based on the comparison with the reference dataset of signal properties, compare the microbe profile of the user with at least one reference microbe profile, and recommend to the user at least one of the plurality of treatment agents based on the comparison with the at least one reference microbe profile.

At least one microbe sampling unit 1520 includes a microbe-capture region configured to capture one or more types of microbes from a body surface of a user. In an aspect, the microbe-capture region of the at least one microbe sampling unit 1520 is configured to capture one or more types of microbes from a skin surface of the user. In an aspect, the microbe-capture region of the at least one microbe sampling unit 1520 is configured to capture one or more types of microbes from a gastrointestinal surface of the user. In an aspect, the microbe-capture region of the at least microbe sampling unit 1520 is configured to capture one or more types of microbes from a mucosal surface, a vaginal surface, a nasal surface, or a mouth surface of the user.

In an aspect, the microbe-capture region of at least one microbe sampling unit 1520 includes a charged surface. In an aspect, the microbe-capture region of at least one microbe sampling unit 1520 includes at least one of an adhesive, an absorbent, or an adsorbent. In an aspect, the microbe-capture region of at least one microbe sampling unit 1520 includes a biomolecule-binding polymer. In an aspect, the microbe-capture region of at least one microbe sampling unit 1520 includes a gel. Non-limiting examples of materials for a microbe-capture region have been described above herein.

In an aspect, the microbe-capture region of at least one microbe sampling unit 1520 includes a plurality of specific microbe-binding elements. In an aspect, at least one of the plurality of specific microbe-binding elements includes a specific microbe-binding antibody. In an aspect, at least one of the plurality of specific microbe-binding elements includes a specific microbe-binding oligonucleotide. In an aspect, at least one of the plurality of specific microbe-binding elements includes a protein, a peptide, a lectin, a carbohydrate, RNA, DNA, an anti-16S rRNA ligand, an aptamer, a synthetic ligand, or a mimetic binding element. In an aspect, the microbe-capture region includes a plurality of specific microbe-binding elements of at least one first type and a plurality of specific microbe-binding elements of at least one second type. Non-limiting examples of specific microbe-binding elements have been described above herein.

In an aspect, the microbe-capture region of at least one microbe sampling unit 1520 includes a plurality of signal-generating complexes. In an aspect, at least one of the plurality of signal-generating complexes comprises an optical signal-generating complex, a fluorescing signal-generating complex, an electromagnetic signal-generating complex, a radio signal-generating complex, an electrical current signal-generating complex, an acoustic signal-generating complex, or a magnetic signal-generating complex. In an aspect, each of the plurality of signal-generating complexes includes at least one signal-generating element operably coupled to at least one specific microbe-binding element, the at least one signal-generating element configured to emit one or more signals in response to contact with at least one type of microbe by the operably coupled at least one specific microbe-binding element. In an aspect, the microbe-capture region includes a plurality of signal-generating complexes of at least one first type able to emit at least one first signal type in response to at least one first type of microbe and a plurality of signal-generating complexes of at least one second type able to emit at least one second signal type in response to at least one second type of microbe. Non-limiting examples of signal-generating complexes including signal-generating element have been described above herein.

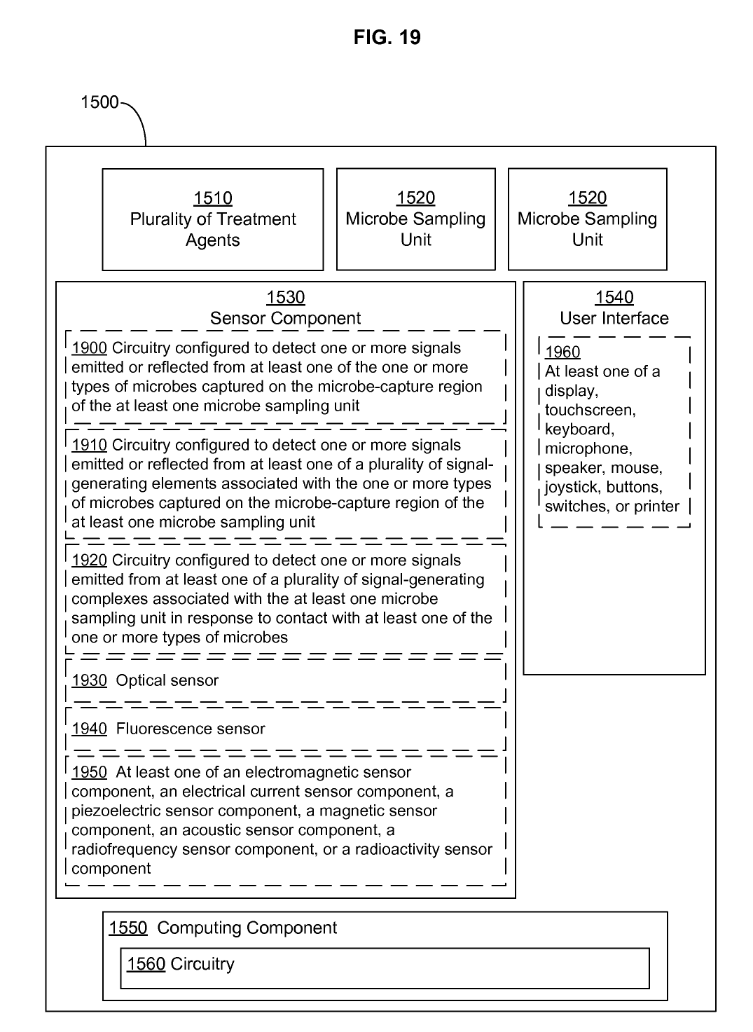

With reference to FIG. 19, kiosk 1500 includes at least one sensor component 1530 including circuitry configured to detect one or more signals emitted or reflected from the microbe-capture region of at least one microbe sampling unit 1520. In an aspect, the at least one microbe sampling unit is fed into the kiosk through a receiving component, e.g., receiving component 1710 of FIG. 17, and onto a conveyor which moves the microbe sampling unit into the vicinity of the at least one sensor component. In an aspect, at least one sensor component 1530 includes circuitry 1900 configured to detect one or more signals emitted or reflected from at least one of the one or more types of microbes captured on the microbe-capture region of at least one microbe sampling unit 1520. In an aspect, at least one sensor component 1530 includes circuitry 1910 configured to detect one or more signal emitted or reflected from at least one of a plurality of signal-generating elements associated with the one or more types of microbes captured on the microbe-capture region of the at least one microbe sampling unit 1520. In an aspect, at least one sensor component 1530 includes circuitry 1920 configured to detect one or more signals emitted from at least one of a plurality of signal-generating complexes associated with the at least one microbe sampling unit 1520 in response to contact with at least one of the one or more types of microbes. In an aspect, at least one sensor component 1530 includes at least one optical sensor 1930. In an aspect, at least one sensor component 1530 includes at least one fluorescence sensor 1940. In an aspect, at least one sensor component 1530 includes at least one of an electromagnetic sensor component, an electrical current sensor component, a piezoelectric sensor component, a magnetic sensor component, an acoustic sensor component, a radiofrequency sensor component, or a radioactivity sensor component 1950. Non-limiting examples of sensor components have been described above herein.

Kiosk 1500 further includes user interface 1540. User interface 1540 is operably coupled to computing component 1550 and includes one or more input components and/or output components for use by a user to interface with kiosk 1500. As shown in block 1960 of FIG. 19, user interface 1540 can include at least one of a display, touchscreen, keyboard, microphone, speaker, mouse, joystick, buttons, switches, or printer. One or more input components, e.g., a touchscreen display and/or a microphone, can be used to enter information e.g., user information, into the kiosk. In some embodiments, the user interface is user-driven. For example, the user inputs data or operating conditions into the kiosk using the user interface, e.g., a touchscreen display. In some embodiments, the user interface, e.g., a switch, is circuitry driven. For example, an on/off switch may be toggled based on proximity of a user to the kiosk. One or more output components, e.g., a touchscreen display, speaker, and/or printer, can be used to view output results of processed information. For example, the user interface may be used to report to a user a microbe profile including a spatial distribution and/or an identity of one or more types of microbes on a body surface, e.g., a skin surface, of the user and/or the recommended at least one of the plurality of treatment agents. Non-limiting examples of user interface input and output components have been described above herein.

Kiosk 1500 further includes computing component 1550. Computing component 1550 includes a processor and is operably coupled to one or more dispensers 1515, user interface 1540, and at least one sensor component 1530. Computing component 1550 further includes circuitry 1560. Circuitry 1560 includes circuitry configured to receive the sensor output from the at least one sensor component, the sensor output including information associated with at least one property of the detected one or more signals emitted or reflected from the microbe-capture region of the at least one microbe sampling unit; circuitry configured to compare the at least one property of the detected one or more signals emitted or reflected from the microbe-capture region of the at least one microbe sampling unit with a reference dataset of signal properties; circuitry configured to generate a microbe profile of the user based on the comparison with the reference dataset of signal properties; circuitry configured to compare the microbe profile of the user with at least one reference microbe profile; circuitry configured to recommend at least one of the plurality of treatment agents to the user based on the comparison with the at least reference microbe profile; and circuitry configured to send a signal to at least one of the one or more dispensers to dispense the recommended at least one of the plurality of treatment agents from the kiosk to the user.

Computing component 1550 further includes circuitry configured to execute one or more instructions for operating the components of the kiosk, e.g., the one or more dispensers, the at least sensor component and the user interface. In an aspect, computing component 1550 includes circuitry configured to execute one or more instructions for operating any or all other components incorporated into the kiosk, e.g., a transmission unit, a microbe sampling unit dispensing component, a receiving component, a treatment agent dispensing component, a feeding mechanism, at least one reservoir, and at least one motor. The computing component includes circuitry configured to execute one or more instructions for receiving the sensor output from the at least one sensor component, the sensor output including information associated with at least one property of the detected one or more signals emitted or reflected from the microbe-capture region of the at least one microbe sampling unit; one or more instructions for comparing the at least one property of the detected one or more signals emitted or reflected from the microbe-capture region of the at least one microbe sampling unit with a reference dataset of signal properties; one or more instructions for generating a microbe profile of the user based on the comparison with the reference dataset of signal properties; one or more instructions for generating the microbe profile of the user with at least one reference microbe profile; one or more instructions for recommending at least one of the plurality of treatment agents to the user based on the comparison with the at least reference microbe profile; and one or more instructions for sending a signal to at least one of the one or more dispensers to dispense the recommended at least one of the plurality of treatment agents from the kiosk to the user.

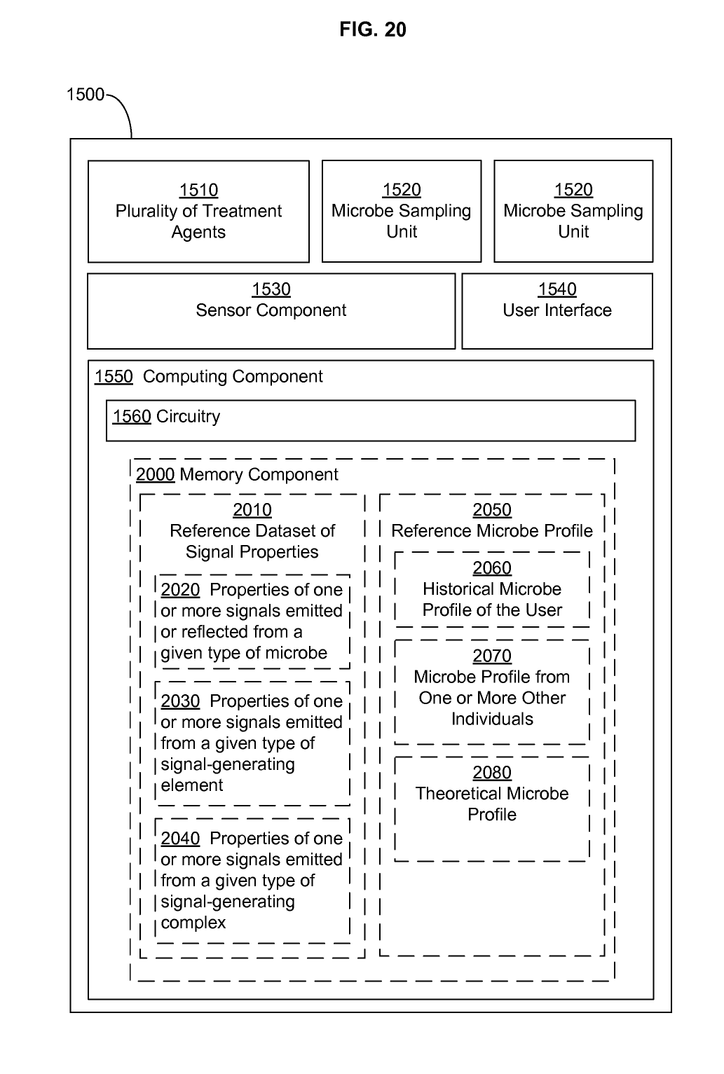

FIG. 20 shows further aspects of computing component 1550 of kiosk 1500. In an aspect, computing component 1550 includes circuitry configured to compare the at least one property of the detected one or more signals emitted or reflected from the microbe-capture region of the at least one microbe sampling unit with a reference dataset of signal properties. In an aspect, the at least one property of the detected one or more signals emitted or reflected from the microbe-capture region of the microbe sampling unit includes at least one of an optical property, a fluorescent property, a magnetic property, an electrical property, an electromagnetic property, or a radiofrequency property. In an aspect, reference dataset of signal properties 2010 is included in a memory component 2000 of computing component 1550. In an aspect, reference dataset of signal properties 2010 includes properties 2020 of one or more signals emitted or reflected from a given type of microbe. In an aspect, reference dataset of signal properties 2010 includes properties 2030 of one or more signals emitted from a given type of single-generating element. In an aspect, reference dataset of signal properties 2010 includes properties 2040 of one or more signals emitted from a given type of signal-generating complex.

In an aspect, computing component 1550 includes circuitry configured to compare the microbe profile of the user with at least one reference microbe profile. In an aspect, at least one reference microbe profile 2050 is included in memory component 2000 of computing component 1550. In an aspect, at least one reference microbe profile 2050 includes at least one historical microbe profile of the user 2060. In an aspect, at least one reference microbe profile 2050 includes at least one microbe profile from one or more other individuals 2070. In an aspect, at least one reference microbe profile 2050 includes theoretical microbe profile 2080.

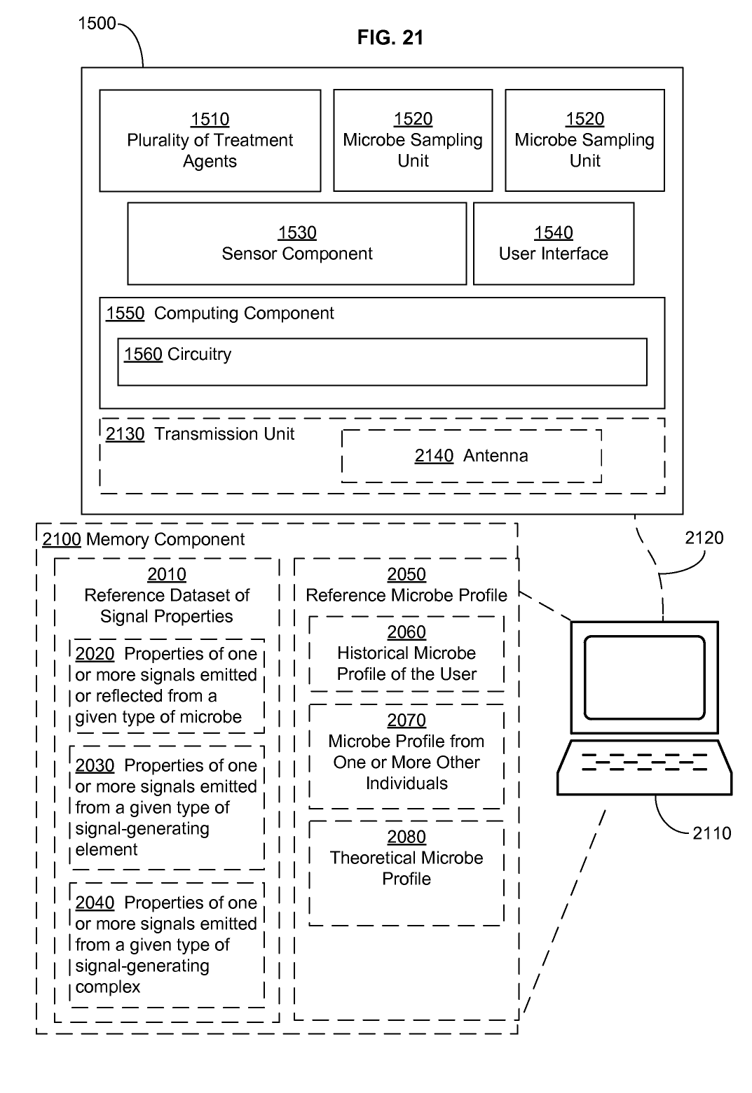

In an aspect, the reference dataset of signal properties and the at least one reference microbe profile are incorporated into a memory component of the computing component of the kiosk. In an aspect, as shown in FIG. 21, the reference dataset of signal properties 2010 and/or at least one reference microbe profile 2050 are accessed from a remote computing device 2110 through a communication link 2120. In an aspect, reference dataset of signal properties 2010 including at least one of properties 2020 of one or more signals emitted from a given type of microbe, properties 2030 of one or more signals emitted from a given type of signal-generating element, and/or properties 2040 of one or more signals emitted from a given type of signal-generating complex are stored in memory component 2100 of remote computing device 2110. In an aspect, at least one reference microbe profile 2050 including at least one historical microbe profile of the user 2060, at least one microbe profile from one or more other individuals 2070, and/or at least one theoretical microbe profile 2080 are stored in memory component 2100 of remote computing device 2110. In an aspect, the remote computing device is associated with at least one of a medical practice, a laboratory, a supplier, a retailer, a manufacturer, or other like entity. In an aspect, the remote computing device is associated with a remote server, a cloud-based server, a web-based server. In an aspect, the communication link includes a wired connection, e.g., a cable communication. In an aspect, the communication link includes a wireless connection. In an aspect, kiosk 1500 further includes transmission unit 2130 including an antenna 2140. Non-limiting aspects of a transmission unit have been described above herein.

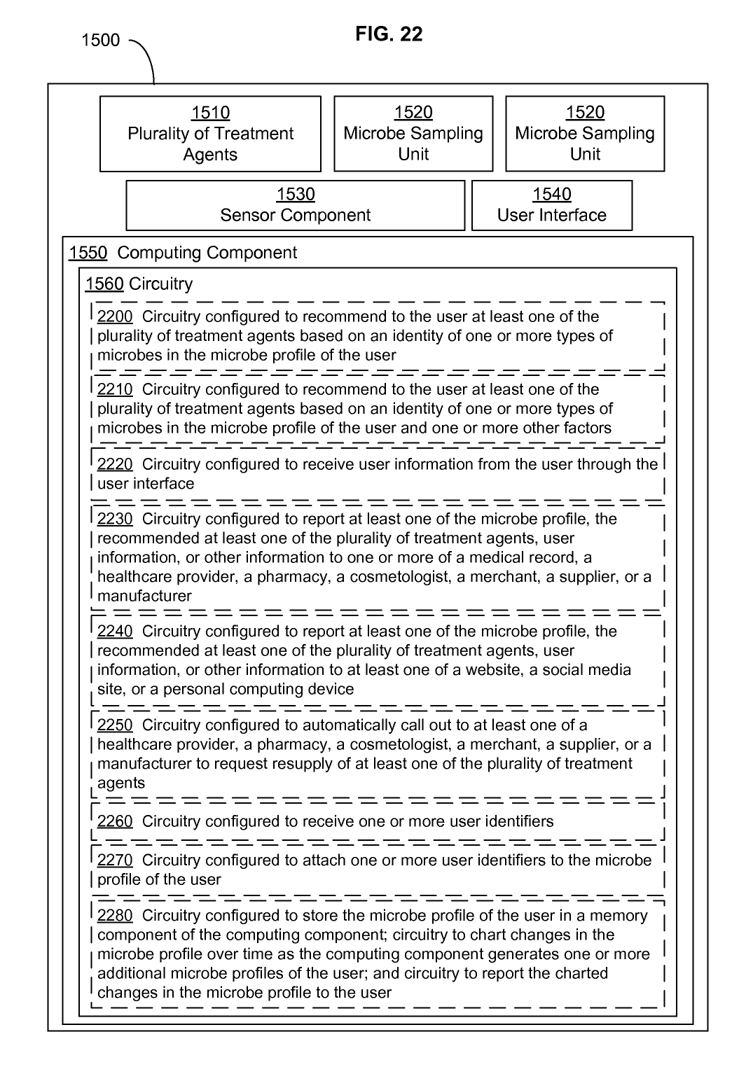

FIG. 22 shows further aspects of a kiosk. Kiosk 1500 includes computing component 1550 with circuitry 1560. In an aspect, circuitry 1560 of computing component 1550 includes circuitry 2200 configured to recommend to the user at least one of the plurality of treatment agents based on an identity of one or more types of microbes in the microbe profile of the user. In an aspect, circuitry 1560 of computing component 1550 includes circuitry 2210 configured to recommend to the user at least one of the plurality of treatment agents based on an identity of one or more types of microbes included in the microbe profile of the user and one or more other factors. In an aspect, the one or more other factors include one or more of age, gender, ethnicity, skin characteristics, geographical location, medical history, or co-morbidities. In an aspect, the one or more other factors include at least one user preference. In an aspect, circuitry 1560 of computing component 1550 includes circuitry 2220 configured to receive user information from the user through the user interface. In an aspect, the user information includes at least one of age, gender, ethnicity, skin characteristics, geographical location, medical history, co-morbidities, or user preferences.

In an aspect, circuitry 1560 of computing component 1550 includes circuitry 2230 configured to report at least one of the microbe profile, the recommended at least one of the plurality of treatment agents, user information, or other information to one or more of a medical record, a healthcare provider, a pharmacy, a cosmetologist, a merchant, a supplier, or a manufacturer. In an aspect, circuitry 1560 of computing component 1550 includes circuitry 2240 configured to report at least one of the microbe profile, the recommended at least one of the plurality of treatment agents, user information, or other information to at least one of a website, a social media site, or a personal computing device. In an aspect, circuitry 1560 of computing component 1550 includes circuitry 2250 configured to automatically call out to at least one of a healthcare provider, a pharmacy, a cosmetologist, a merchant, a supplier, or a manufacturer to request resupply of at least one of the plurality of treatment agents.

In an aspect, circuitry 1560 of computing component 1550 includes circuitry 2260 configured to receive one or more user identifiers. For example, the computing component can include circuitry configured to receive an identification code, e.g., an alphanumeric code, from the user through the user interface, e.g., a touchscreen display or microphone. For example, the computing component can include circuitry configured to receive one or more biometric measures from the user through the user interface of the kiosk. For example, the biometric measures can include an image of the user, a voice print or finger print of the user, a retinal scan of the user, and the like. In an aspect, circuitry 1560 of computing component 1550 includes circuitry 2270 configured to attach one or more user identifiers to the microbe profile of the user. For example, the computing component can include circuitry to attach an identification code, e.g., an alphanumeric code, to the microbe profile of the user. In this manner, the user may return to the kiosk at a later date to access a historical microbe profile. In an aspect, circuitry 1560 of computing component 1550 includes circuitry 2280 configured to store the microbe profile in a memory component of the computing component; circuitry configured to chart changes in the microbe profile over time as the computing component generates one or more additional microbe profiles of the user; and circuitry configured to report the charted changes in the microbe profile to the user.

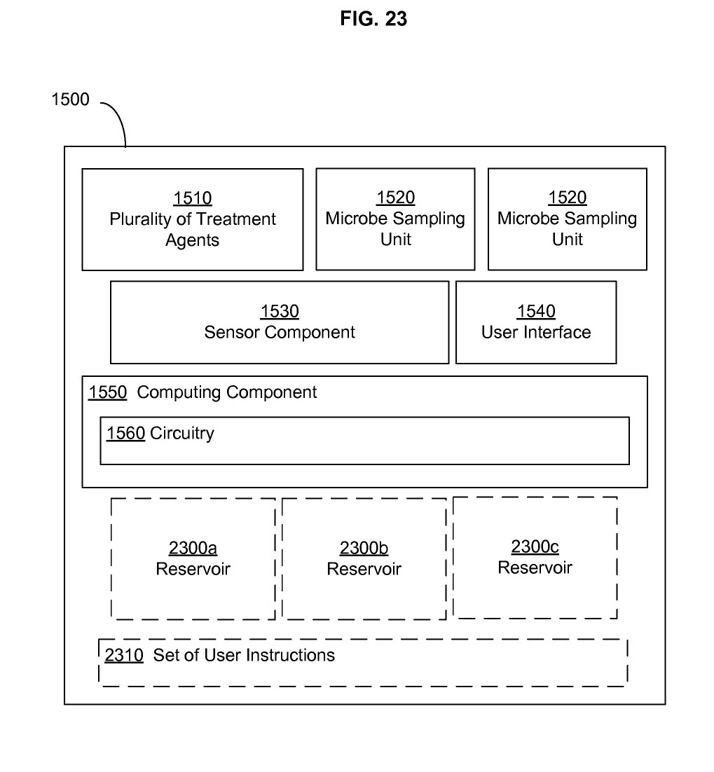

FIG. 23 illustrates further aspects of a kiosk. In some embodiments, kiosk 1500 includes one or more reservoirs 2300a, 2300b, and 2300c. In an aspect, at least one of the one or more reservoirs includes a plurality of at least one type of signal-generating elements. Non-limiting examples of signal-generating elements have been described above herein. In an aspect, at least one of the one or more reservoirs includes at least one of a buffer, a detergent solution, a reagent solution, or a wash solution, non-limiting examples of which have been described above herein.

In an aspect, a kiosk such as described herein further includes a set of user instructions 2310 for using the kiosk or a component of the kiosk. In an aspect, the set of user instructions includes at least one of one or more instructions for using the kiosk, one or more instructions for sampling the body surface of the user with the at least one microbe sampling unit, one or more instructions for inserting the at least one microbe sampling unit into at least one receiving component of the kiosk, one or more instructions for interpreting the microbe profile, one or more instructions for applying the recommended at least one of the plurality of treatment agents to the body surface, or one or more instructions for accessing a website. In an aspect, the set of user instructions include written instructions posted on the outside of the kiosk. In an aspect, the set of user instructions are included in the computing component of the kiosk and are provided to the user through one or more of the user interfaces, e.g., through a display, printout, or speaker associated with the kiosk.

### Method

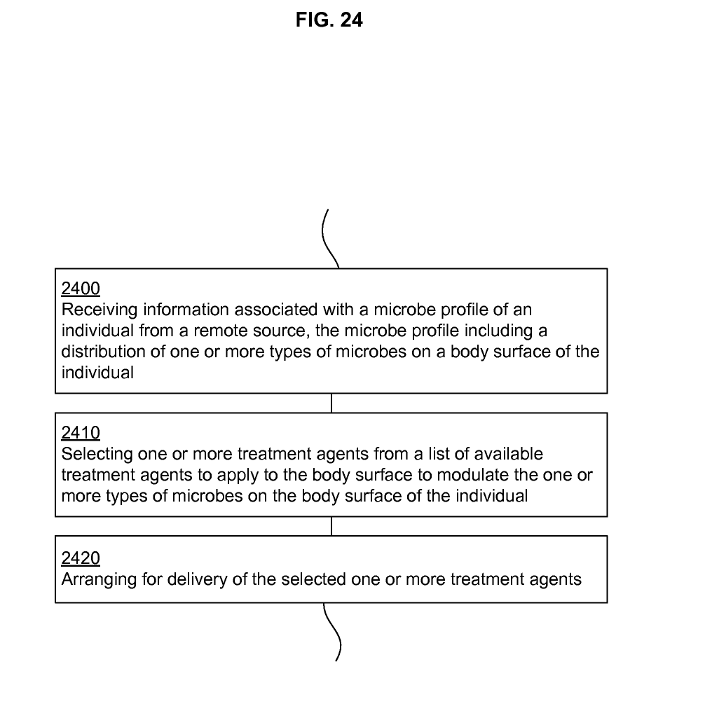

FIG. 24 shows a flowchart of a method for selecting one or more treatment agents based on a received microbe profile and arranging for delivery of the one or more treatment agents. The method includes in block 2400, receiving information associated with a microbe profile of an individual from a remote source, the microbe profile including a distribution of one or more types of microbes on a body surface of the individual; in block 2410, selecting one or more treatment agents from a list of available treatment agents to apply to the body surface to modulate the one or more types of microbes on the body surface of the individual; and in block 2420, arranging for delivery of the selected one or more treatment agents.

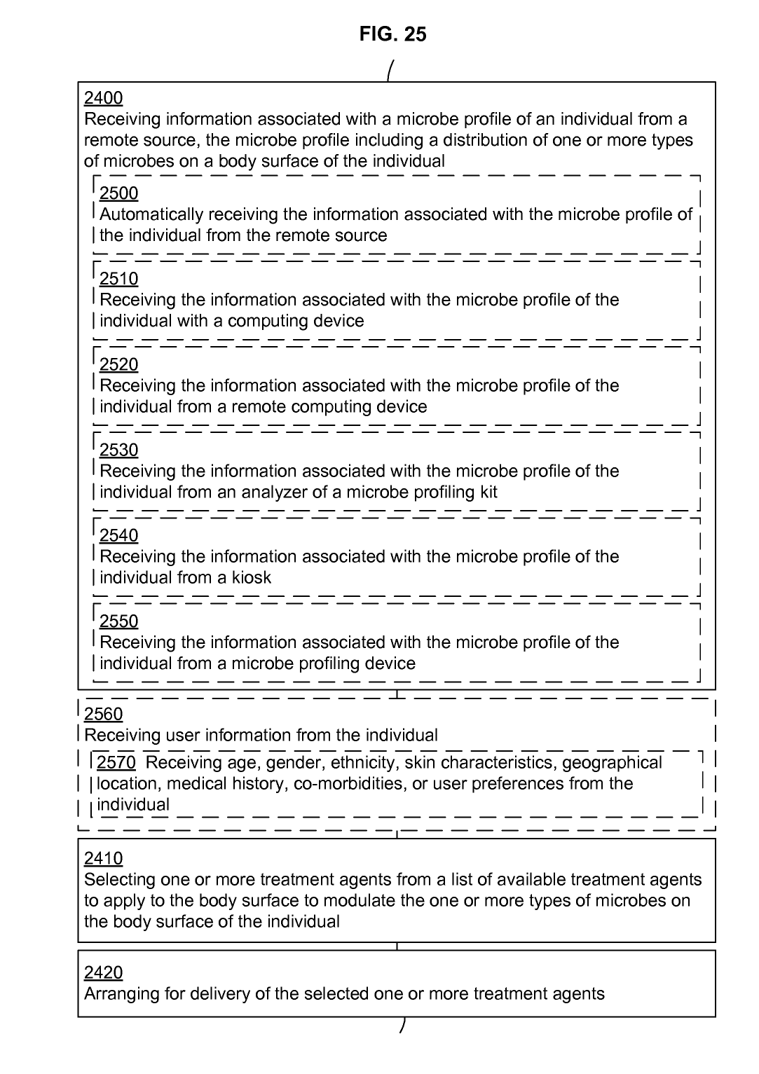

FIG. 25 illustrates further aspects of a method such as described in FIG. 24. In an aspect, the method includes receiving the information associated with the microbe profile of the individual with a computing device, as shown in block 2510. For example, the method can include receiving the information associated with the microbe profile of the individual with a computing device, for example a desktop computer, personal computer, server, or other computing device, e.g., associated with a medical practice, a pharmacy, a retailer, a supplier, a wholesaler, or a manufacturer. For example, the method can include receiving the information associated with the microbe profile of the individual with a computer associated with a service center associated with a retailer, supplier, wholesaler, or manufacturer.

In an aspect, receiving information associated with a microbe profile of an individual from a remote source includes automatically receiving the information associated with the microbe profile of the individual from the remote source, as shown in block 2500. In an aspect, the method includes receiving the information associated with the microbe profile of the individual from a remote computing device, as shown in block 2520. In an aspect, the method includes receiving the information associated with the microbe profile of the individual from a remote computing device associated with a residence, a medical practice, pharmacy, cosmetic counter, or kiosk. In an aspect, the method includes receiving the information associated with the microbe profile of the individual from an analyzer of a microbe profiling kit, as shown in block 2530. For example, the analyzer of a microbe profiling kit, non-limiting examples of which have been described herein, can wirelessly transmit the information associated with the microbe profile of the individual to a computing device associated with a medical practice, pharmacy, retailer, supplier, wholesaler, or manufacturer. In an aspect, the method includes receiving the information associated with the microbe profile of the individual from a kiosk, as shown in block 2540. In an aspect, the method includes receiving the information associated with the microbe profile of the individual from a microbe profiling device, as shown in block 2550. Various microbe profiling devices for generating a microbe profile and transmitting information associated with said microbe profile have been described. See, e.g., U.S. patent application Ser. No. 14/091,832, which is incorporated herein by reference.

In an aspect, the method further includes receiving user information from the individual, as shown in block 2560. In an aspect, the method includes receiving age, gender, ethnicity, skin characteristics, geographical location, medical history, co-morbidities, or user preference from the individual, as shown in block 2570. In an aspect, receiving user information includes receiving user information from at least one of a remote computing device, an analyzer of a microbe profiling kit, a kiosk, or a microbe profiling device. In an aspect, the method includes receiving the user information with a computing device associated with a medical practice, a pharmacy, a retailer, a wholesaler, a supplier, or a manufacturer.

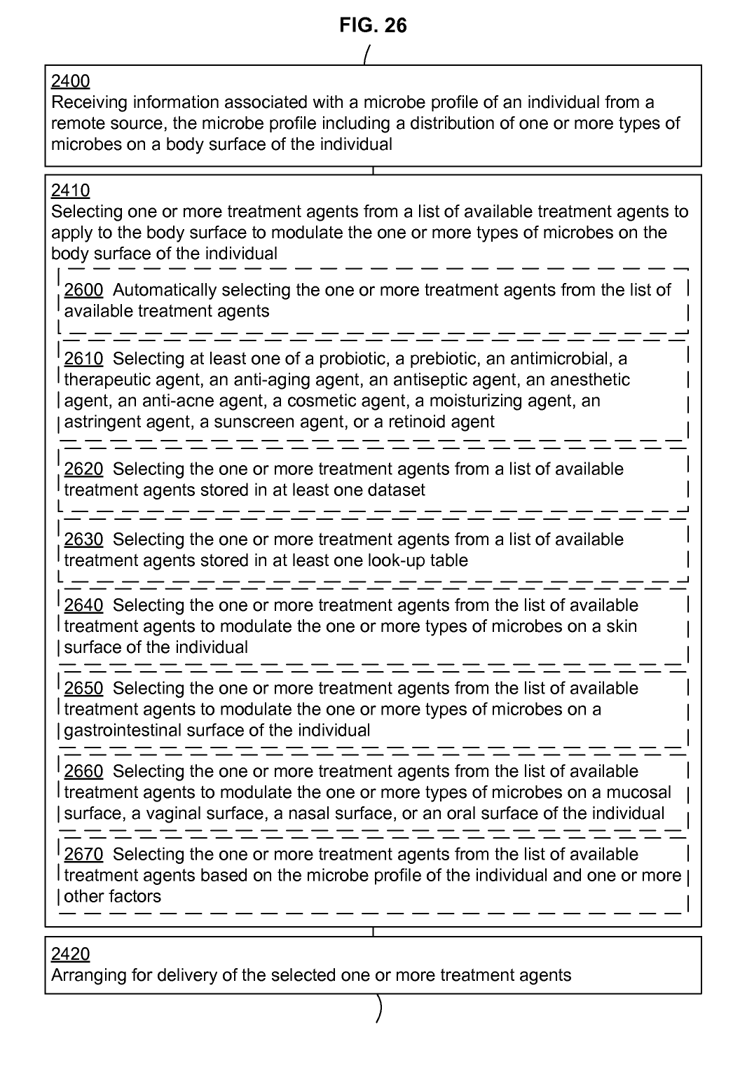

FIG. 26 illustrates further aspects of a method such as that shown in FIG. 24. In an aspect, the method includes selecting one or more treatment agents from a list of available treatment agents to apply to a body surface to modulate the one or more types of microbe on the body surface of the individual. In an aspect, modulate includes promoting the growth of one or more types of microbes. In an aspect, modulate includes inhibiting the growth of one or more types of microbes. In an aspect, modulate includes modulating the environment, e.g., pH or moisture, on the body surface to modulate the one or more types of microbes. In an aspect, selecting one or more treatment agents from a list of available treatment agents to apply to the body surface to modulate one or more types of microbes on the body surface of the individual include in block 2600 automatically selecting the one or more treatment agents from the list of available treatment agents. For example, the method can be implemented on a computing device with a processor that automatically consults at least one dataset, database, or look-up table including the list of treatment agents relative to types of microbes that is accessible to the computing device. In an aspect, the method includes selecting at least one of a probiotic, a prebiotic, an antimicrobial, a therapeutic agent, an anti-aging agent, an antiseptic agent, an anesthetic agent, an anti-acne agent, a cosmetic agent, a moisturizing agent, an astringent agent, a sunscreen agent, or a retinoid agent, as shown in block 2610. Non-limiting examples of treatment agents have been described above herein. In an aspect, the method includes selecting the one or more treatment agents from a list of available treatment agents stored in at least one dataset, as shown in block 2620. In an aspect, the list of available treatment agents stored in the at least one dataset is matched with a list of microbe types. In an aspect, the method includes selecting the one or more treatment agents from a list of available treatment agents stored in at least one look-up table, as shown in block 2630. In an aspect, the list of available treatment agents stored in the at least one look-up table is matched with a list of microbe types.

In an aspect, the method includes selecting the one or more treatment agents from the list of available treatment agents to modulate the one or more types of microbes on a skin surface of the individual, as shown in block 2640. In an aspect, the method includes selecting the one or more treatment agents from the list of available treatment agents to modulate the one or more types of microbes on a gastrointestinal surface of the individual, as shown in block 2650. In an aspect, the method includes selecting the one or more treatment agents from the list of available treatment agents to modulate the one or more types of microbes on a mucosal surface, a vaginal surface, a nasal surface, or an oral surface of the individual, as shown in block 2660.

In an aspect, the method further includes selecting the one or more treatment agents from the list of available treatment agents based on the microbe profile of the individual and one or more other factors, as shown in block 2670 of FIG. 26. In an aspect, the method includes selecting the one or more treatment agents from the list of available treatment agents based on the microbe profile of the individual and one or more of age, gender, ethnicity, skin characteristics, geographical location, medical history, user preferences, or co-morbidities.

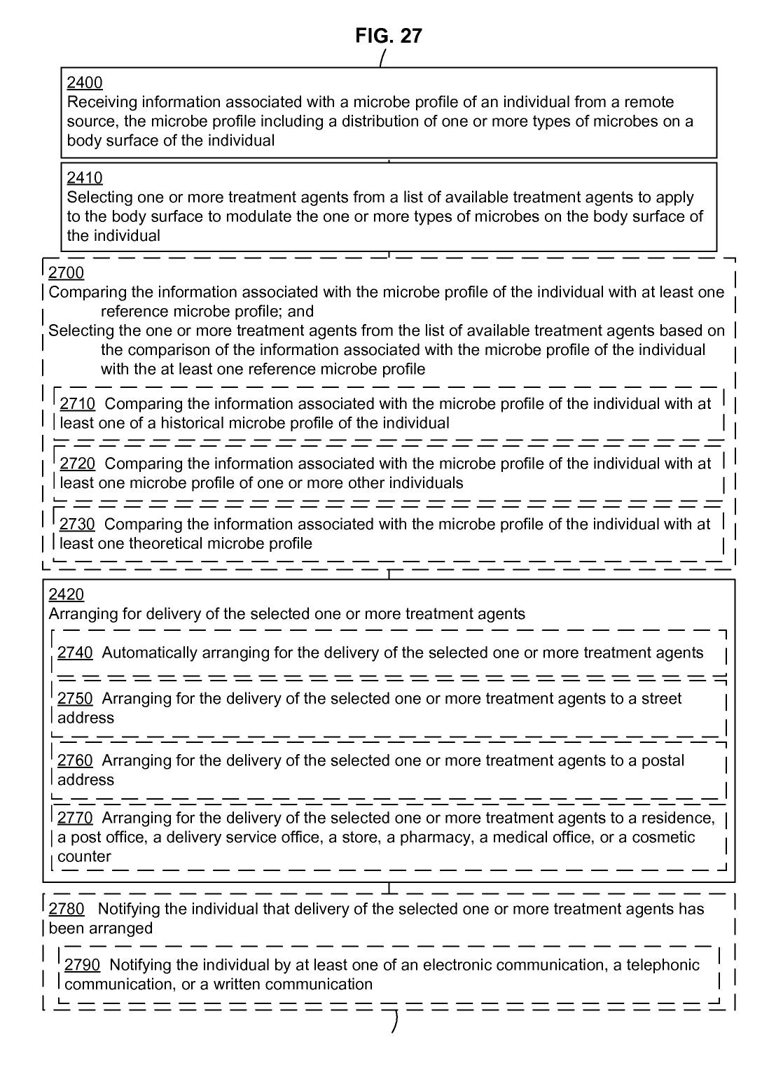

FIG. 27 illustrates further aspects of a method such as shown in FIG. 24. In an aspect, the method includes comparing the information associated with the microbe profile of the individual with at least one reference microbe profile; and selecting the one or more treatment agents from the list of available treatment agents based on the comparison of the information associated with the microbe profile of the individual with the at least one reference microbe profile, as shown in block 2700. In an aspect, the method includes comparing the information associated with the microbe profile of the individual with at least one of a historical microbe profile of the individual, as shown in block 2710. For example, the method can include comparing a current microbe profile of the individual with a historical microbe profile of the individual generated at a previous point in time, e.g., at an earlier age, prior to the onset of a condition, and/or prior to onset of a treatment. In an aspect, the method includes comparing the information associated with the microbe profile of the individual with at least one microbe profile of one or more other individuals, as shown in block 2720. For example, the method can include comparing a current microbe profile of the individual with at least one microbe profile representing an average or normalized population of individuals, an idealized population of individuals, or an admired individual. In an aspect, the method includes comparing the information associated with the microbe profile of the individual with at least one theoretical microbe profile, as shown in block 2730.

The method includes arranging for delivery of the selected one or more treatment agents. For example, the method includes arranging for delivery of the selected one or more treatment agents by way of the Postal Service, shipping service, e.g., FedEx or UPS, or courier. In an aspect, the method includes automatically arranging for the delivery of the selected one or more treatment agents, as shown in block 2740. For example, a computing device executing the method described herein can automatically arrange for delivery of the selected one or more treatment agents by requesting the selected items be pulled from inventory, labeled for shipment, and loaded for delivery. In an aspect, the method includes arranging for the delivery of the selected one or more treatment agents to a street address, as shown in block 2750. For example, the method can include arranging delivery of the selected one or more treatment agents to a street address of at least one of the individual's residence or workplace. For example, the method can include arranging delivery of the selected one or more treatment agents to a street address of at least one of a medical practice, pharmacy, or retail store for pick up by the individual. In an aspect, the method includes arranging for the delivery of the selected one or more treatment agents to a postal address, e.g., a post office box, as shown in block 2760. In an aspect, the method includes arranging for the delivery of the selected one or more treatment agents to a residence, a post office, a delivery service office, a store, a pharmacy, a medical office, or a cosmetic counter, as shown in block 2770.

In an aspect, the method further includes notifying the individual that delivery of the selected one or more treatment agents has been arranged, as shown in block 2780. In an aspect, the method includes notifying the individual by at least one of an electronic communication, a telephonic communication, or a written communication, as shown in block 2790. For example, the method can include notifying the individual by way of an e-mail communication or a text message. For example, the method can include notifying the individual by way of an automated telephone call. For example, the method can include notifying the individual by way of a postcard or letter sent through the mail.

### System

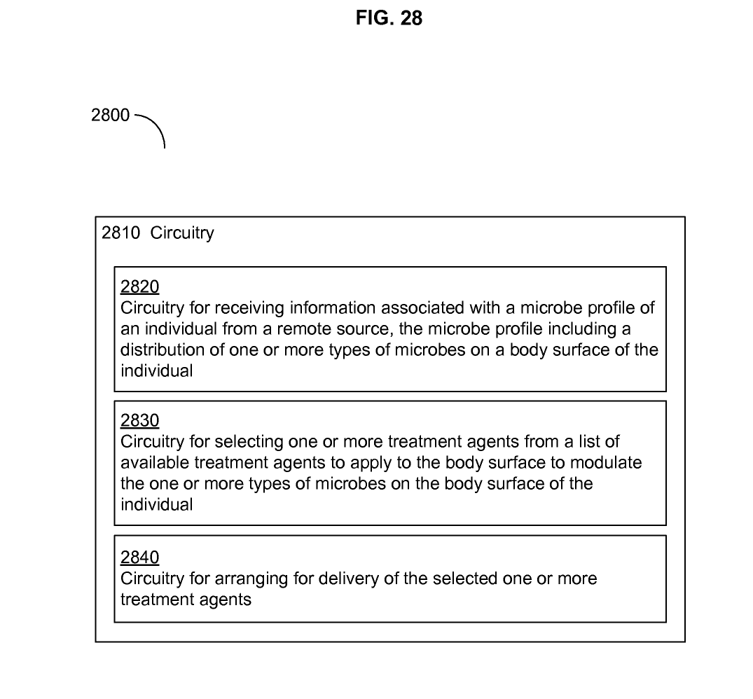

With reference to FIG. 28, described is a system including circuitry configured for selecting and arranging for delivery of one or more treatment agents. System 2800 includes circuitry 2810. Circuitry 2810 includes circuitry 2820 for receiving information associated with a microbe profile of an individual from a remote source, the microbe profile including a distribution of one or more types of microbes on a body surface of the individual. Circuitry 2810 includes circuitry 2830 for selecting one or more treatment agents from a list of available treatment agents to apply to the body surface to modulate the one or more types of microbes on the body surface of the individual. Circuitry 2810 includes circuitry 2840 for arranging for delivery of the selected one or more treatment agents.

In an aspect, a system for selecting and arranging for delivery of one or more treatment agents includes a computing device. FIG. 29 shows system 2900 including computing device 2910. In an aspect, computing device 2910 includes circuitry 2920 including circuitry 2930 for receiving information associated with a microbe profile of an individual form a remote source, the microbe profile including a distribution of one or more types of microbes on a body surface of the individual, circuitry 2940 for selecting one or more treatment agents from a list of available treatment agents to apply to the body surface to modulate the one or more types of microbes on the body surface of the individual, and circuitry 2950 for arranging for delivery of the selected one or more treatment agents.

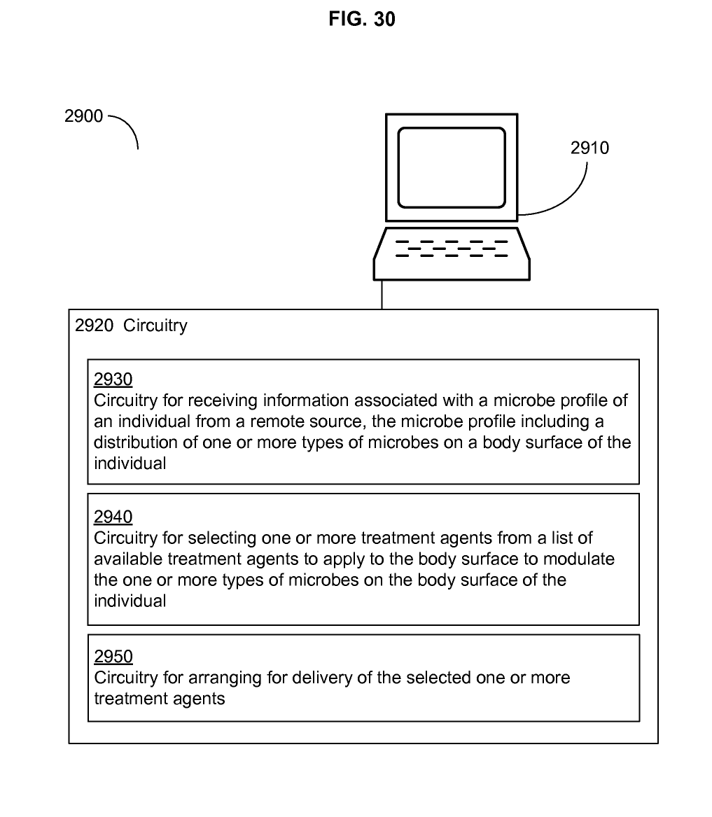

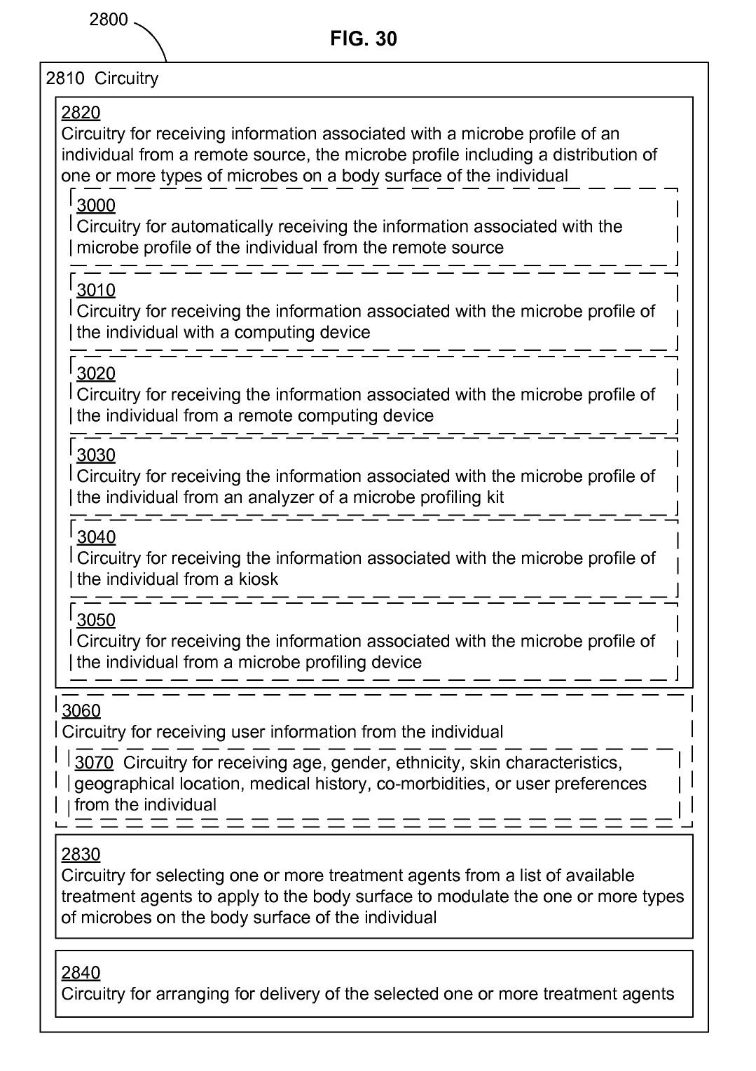

FIG. 30 shows further aspects of a system such as shown in FIG. 28. In an aspect, circuitry 2810 of system 2800 includes circuitry 3000 for automatically receiving the information associated with the microbe profile of the individual from the remote source. In an aspect, circuitry 2810 includes circuitry 3010 for receiving the information associated with the microbe profile of the individual with a computing device. In an aspect, circuitry 2810 includes circuitry 3020 for receiving the information associated with the microbe profile of the individual from a remote computing device. In an aspect, the remote computing device is located in a residence, a medical facility, a retail center, or a pharmacy. In an aspect, the remote computing device is operably coupled to an analyzer, a microbe profiling device, a microbe profiling system, a microbe sampling device, or a kiosk. For example, the system can include circuitry for receiving the information associated with the microbe profile of the individual from a remote computing device associated with a kiosk located in a retail center, e.g., a shopping mall. In an aspect, circuitry 2810 includes circuitry 3030 for receiving the information associated with the microbe profile of the individual from an analyzer of a microbe profiling kit. In an aspect, circuitry 2810 includes circuitry 3040 for receiving the information associated with the microbe profile of the individual from a kiosk. In an aspect, circuitry 2810 includes circuitry 3050 for receiving the information associated with the microbe profile of the individual from a microbe profiling device.

In an aspect, system 2800 further includes circuitry 3060 for receiving user information from the individual. In an aspect, circuitry 3060 includes circuitry 3070 for receiving age, gender, ethnicity, skin characteristics, geographical location, medical history, co-morbidities, or user preferences from the individual. For example, the system can include circuitry to receive the individual's age, gender, and address for use in selecting and arranging for delivery of the one or more treatment agents.

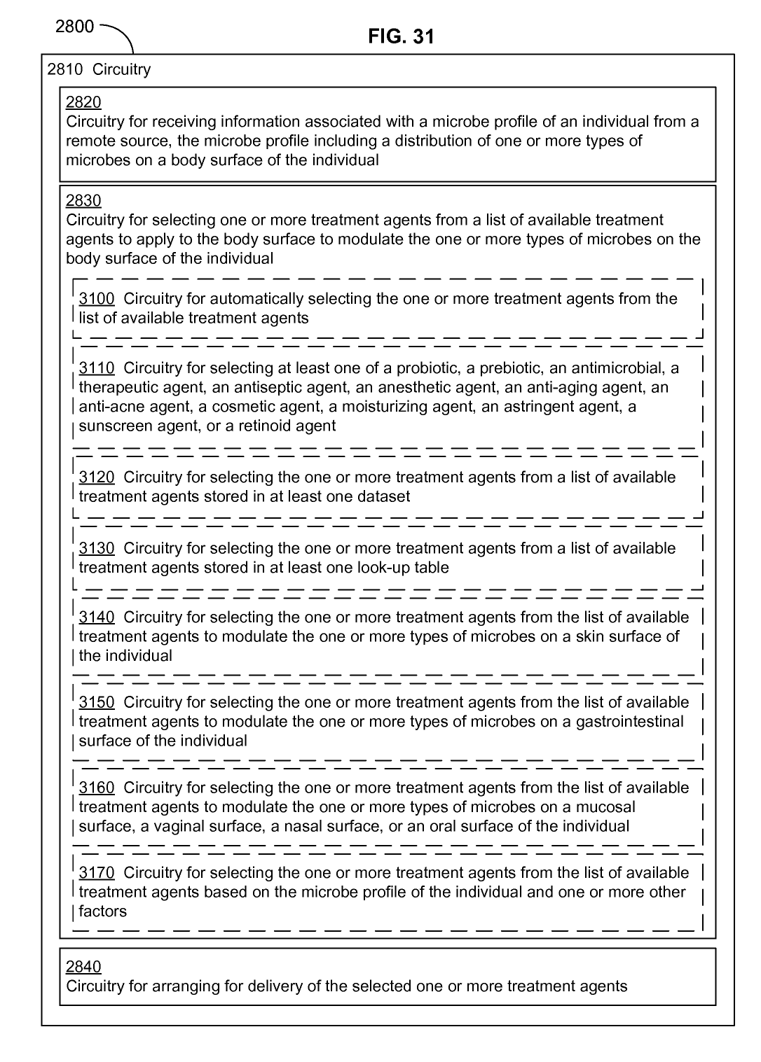

FIG. 31 illustrates further aspects of a system such as shown in FIG. 28. System 2800 includes circuitry 2830 for selecting one or more treatment agents from a list of available treatment agents to apply to the body surface to modulate the one or more types of microbes on the body surface of the individual. In an aspect, circuitry 2830 further includes circuitry 3100 for automatically selecting the one or more treatment agents from the list of available treatment agents. In an aspect, circuitry 2830 includes circuitry 3110 for selecting at least one of a probiotic, a prebiotic, an antimicrobial, a therapeutic agent, an antiseptic agent, an anesthetic agent, an anti-aging agent, an anti-acne agent, a cosmetic agent, a moisturizing agent, an astringent agent, a sunscreen agent, or a retinoid agent from the list of available treatment agents. In an aspect, circuitry 2830 includes circuitry 3120 for selecting the one or more treatment agents from a list of available treatment agents stored in a dataset. In an aspect, circuitry 2830 includes circuitry 3130 for selecting the one or more treatment agents from a list of available treatment agents stored in a look-up table. In an aspect, circuitry 2830 includes circuitry 3140 for selecting the one or more treatment agents from the list of available treatment agents to modulate the one or more types of microbes on a skin surface of the individual. In an aspect, circuitry 2830 includes circuitry 3150 for selecting the one or more treatment agents from the list of available treatment agents to modulate one or more types of microbes on a gastrointestinal surface of the individual. In an aspect, circuitry 2830 includes circuitry 3160 for selecting the one or more treatment agents from the list of available treatment agents to modulate one or more types of microbes on a mucosal surface, a vaginal surface, a nasal surface, or an oral surface of the individual. In an aspect, circuitry 2830 further includes circuitry 3170 for selecting the one or more treatment agents from the list of available treatment agents based on the microbe profile of the individual and one or more other factors. In an aspect, system includes circuitry for selecting the one or more treatment agents from the list of available treatment agents based on the microbe profile of the individual and one or more of age, gender, ethnicity, skin characteristics, geographical location, medical history, co-morbidities, or user preferences.

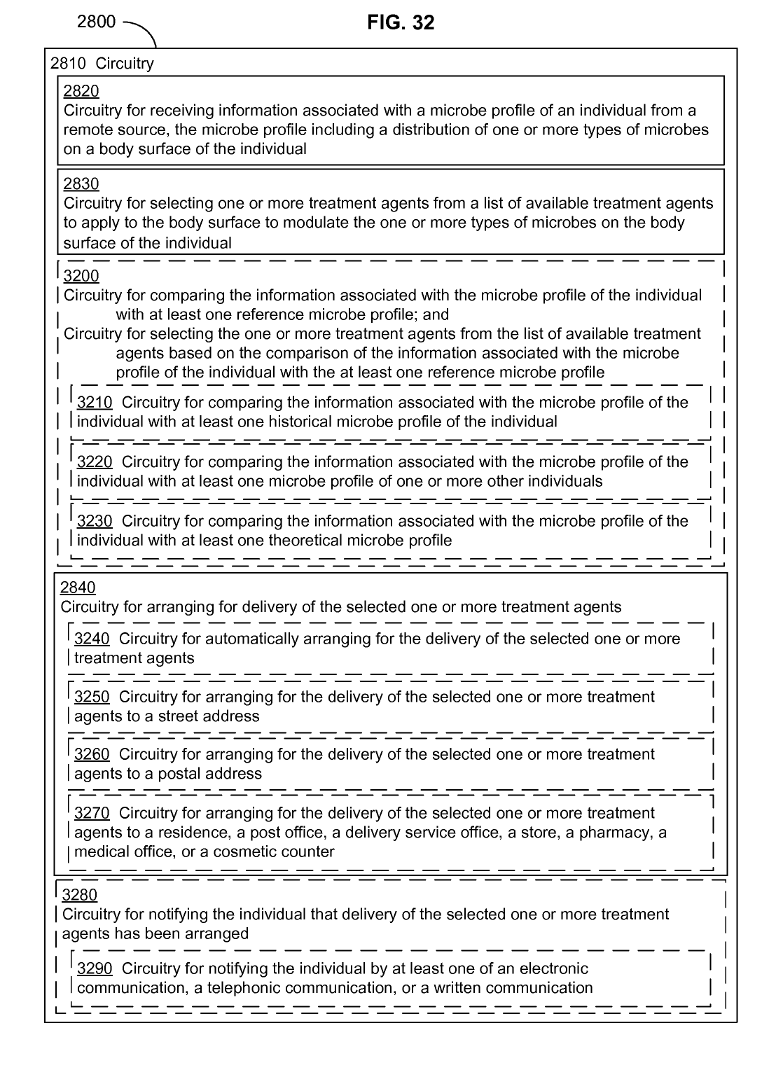

FIG. 32 shows further aspects of a system such as shown in FIG. 28. In an aspect, system 2800 includes circuitry 3200 for comparing the information associated with the microbe profile of the individual with at least one reference microbe profile; and circuitry for selecting the one or more treatment agents from the list of available treatment agents based on the comparison of the information associated with the microbe profile of the individual and with the at least one reference microbe profile. In an aspect, circuitry 3200 includes circuitry 3210 for comparing the information associated with the microbe profile of the individual with at least one historical microbe profile of the individual, e.g., a microbe profile generated at a previous point in time, prior to onset of a condition, and/or prior to onset of treatment for a condition. In an aspect, circuitry 3200 includes circuitry 3220 for comparing the information associated with the microbe profile of the individual with at least one microbe profile of one or more other individuals, e.g., at least one microbe profile from a normalized or average population of individuals, an idealized one or more individuals, or an admired individual. In an aspect, circuitry 3200 includes circuitry 3230 for comparing the information associated with the microbe profile of the individual with at least one theoretical microbe profile.

System 2800 includes circuitry 2840 for arranging for delivery of the selected one or more treatment agents. In an aspect, circuitry 2840 includes circuitry 3240 for automatically arranging for the delivery of the selected one or more treatment agents. In an aspect, circuitry 2840 includes circuitry 3250 for arranging for the delivery of the selected one or more treatment agents to a street address. In an aspect, circuitry 2840 includes circuitry 3260 for arranging for the delivery of the selected one or more treatment agents to a postal address. In an aspect, circuitry 2840 includes circuitry 3270 for arranging for the delivery of the selected one or more treatment agents to a residence, a post office, a delivery service office, a store, a pharmacy, a medical office, or a cosmetic counter. In an aspect, system 2800 further includes circuitry for arranging for delivery of one or more microbe sampling units. For example, the system can include circuitry for arranging for shipment of a resupply of one or more microbe sampling units in response to receiving a recent microbe profile from the individual.

In an aspect, system 2800 further includes circuitry 3280 for notifying the individual that delivery of the selected one or more treatment agents has been arranged. In an aspect, circuitry 3280 includes circuitry 3290 for notifying the individual by at least one of an electronic communication, a telephonic communication, or a written communication.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations can include software or other control structures. Electronic circuitry, for example, may have one or more paths of electrical current constructed and arranged to implement various functions as described herein. In some implementations, one or more media can be configured to bear a device-detectable implementation when such media hold or transmit a device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

Those skilled in the art will recognize that at least a portion of the systems and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors. A data processing system can be implemented utilizing suitable commercially available components, such as those typically found in data computing/communication and/or network computing/communication systems.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory). A sale of a system or method may likewise occur in a territory even if components of the system or method are located and/or used outside the territory.

Further, implementation of at least part of a system for performing a method in one territory does not preclude use of the system in another territory.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

Various non-limiting embodiments are described herein as Prophetic Examples.

### Prophetic Example 1

### A Kit Including a Selection of Treatment Agents, Microbe Sampling Units, and an Analyzer for Assessing and Treating Acne

A kit is described including a selection of treatment agents, several single-use microbe sampling strips, an analyzer, and a booklet including instructions for using the various components of the kit and interpreting the results.

The kit includes treatment agents in individual containers including a topical antibiotic in two forms, Clindamycin phosphate topical gel and Clindamycin topical lotion, and a probiotic including Staphylococcus epidermidis.

The kit further includes a number of single-use microbe sampling strips. Each of the microbe sampling strips includes a thin flexible plastic substrate. One surface of the substrate of the microbe sampling strip is coated with a biocompatible silicone-based pressure sensitive adhesive to form a microbe-capture region, e.g., Dow Corning 7-9700 Soft Skin Adhesive (from, e.g., Dow Corning, Midland, Mich.) is a two-part, platinum-catalyzed silicone elastomer. During manufacture of the microbe sampling strips, the two parts of the adhesive are mixed in a 1:1 ratio and applied to the surface of the microbe sampling strip in a 0.1 mm to 0.5 mm coating, and cured at room temperature per the manufacturer's instructions. The surface including the biocompatible adhesive material is covered with a removable tab.

For use, the tab is removed from the single-use microbe sampling strip to reveal the biocompatible adhesive microbe sampling region. The single-use microbe sampling strip including the biocompatible adhesive is placed in contact with a skin surface of the user, e.g., the skin surface of the face, chest, shoulder, or back, for 5-10 minutes with manually applied pressure, e.g., using hands to press the microbe sampling strip onto the skin surface. The microbe sampling strip with adhered microbes is removed from the skin surface and subjected to analysis with the analyzer. Additional single-use microbe sampling strips from the kit are used to sample microbes from different portions of the skin surface.

The analyzer includes a user interface, e.g., a touchpad display. The user is prompted to enter user information, e.g., age, gender, ethnicity, and skin characteristics (oily, normal, and/or dry). In this example, the user indicates that she has oily skin. The single-use microbe sampling strip is placed on a scanning surface of the analyzer, the surface of the microbe sampling strip including the biocompatible adhesive and adhered microbes facing the sensor components. The analyzer includes a fluorescence spectrometer including a krypton ion laser, a color CCD camera, and a long-pass filter (cutoff wavelength, 550 nm) as described by Koenig & Schneckenburg (in J. Fluorescence (1994) 4:17-40, which is incorporated herein by reference). The excitation wavelength from the krypton laser is 407 nm. The surface of the single-use microbe sampling strip is scanned for autofluorescence signals emitted from any associated microbes.

The analyzer further includes a computing component that includes software for comparing the properties of the signals emitted from the biocompatible adhesive surface with a dataset of reference signal properties including in a memory component of the analyzer. The sensor output from the CCD camera indicates autofluorescence peak emissions of about 580-600, 620, and about 640 nm. The computing component software compares the sensor output with the dataset of reference signal properties and determines that the fluorescent spots or regions of yellow and red corresponding to autofluorescence peak emissions associated with Propionibacterium acnes, a bacterium associated with acne. Additional autofluorescence peak emissions at about 430-450 nm are determined to be associated with sloughed off skin cells (see, e.g., Meerwaldt et al. (2005) J. Am. Soc. Nephrol. 16:3687-3693, which is incorporated herein by reference). The intensity of the autofluorescence peak emissions is used determine a relative abundance of the bacteria.

A microbe profile is generated for the user that includes the spatial distribution of the fluorescent spots associated Propionibacterium acnes. A color scale is used to highlight the abundance of the bacteria detected on the skin surface. The computing device compares the microbe profile of the user with a reference microbe profile of an “average” individual of matched age, gender, and oily skin. The comparison reveals an above “normal” distribution of Propionibacterium acnes and the computing component recommends to the user via the touchscreen display to use the Clindamycin phosphate topical gel (best for oily skin) twice daily for 10 days to inhibit growth of Propionibacterium acnes followed by the probiotic including Staphylococcus epidermidis to regenerate a population of “good bacteria” on the skin surface. The computing component of the analyzer also recommends cleansing the skin surface twice daily with a mild soap.

### Prophetic Example 2

### A Preformed Skin-Covering Material Including a Poly-L-Lysine Microbe-Capture Region for Assessing Microbiota of Skin

A kit is described including a selection of treatment agents, a multi-use microbe sampling mask, an analyzer, and a booklet including instructions for using the various components of the kit and interpreting the results.

The kit includes treatment agents in individual containers. The treatment agents include a probiotic including Staphylococcus epidermidis, a cosmetic foundation for oily skin, a cosmetic foundation for normal skin, and a cosmetic foundation for dry skin. The kit in general is labeled with for different user preferred cosmetic coloring, e.g., “fair,” “beige,” or “golden.”

The kit further includes a pre-formed mask created specifically for the user that substantially conforms to the topography of the user's face. The pre-formed mask of semi-rigid material is created from a digitally rendered model of the skin surface of the user. In some instances, the user submits one or more digital images of the user's face for use in generating the digitally rendered model. Alternatively, the user is instructed to visit a retail outlet, e.g., a professional studio, for three-dimensional imaging. Briefly, two charge-coupled device cameras and a projector connected to a computer are used to scan the surface of the user's face as described in Feng et al. Brit. J. Oral Maxillofacial Surg. (2010) 48:105-109, which is incorporated herein by reference. The user's face is exposed to structured light to collect an optical representation of the skin surface by a point cloud of up to 300,000 points in three-dimensional coordinates. The three-dimensional coordinates are acquired by the computer and used to construct a digitally rendered model of the mask based on the user's facial features using a CAD/CAM software package, e.g., Geomagic Studio (Morrisville, N.C.).

The microbe sampling mask is formed from the digitally rendered model using a commercially available 3D printer. An example of a 3D printer includes the uPrint SE system (from, e.g., Stratasys, Eden Prairie, Minn.). In this example, software associated with the 3D printer system converts an STL format file containing data regarding the digitally rendered model of the mask into deposition paths that guide the extrusion head of the printer, printing the mask layer by layer. The mask, with an overall thickness of 3 mm, is produced from a thermoplastic material, e.g., acrylonitrile butadiene styrene (ABS). The information used to form the mask can be saved for printing additional masks in the future.

The kit further includes a solution of poly-L-lysine (e.g., 0.01% solution, Sigma-Aldrich, St. Louis, Mo.), which is used to generate a microbe-capture region on a surface of the microbe sampling mask. Briefly, the inner surface of the microbe sampling mask intended for contact with the skin surface is coated with a thin layer of poly-L-lysine and allowed to dry either at room temperature or at an elevated temperature, e.g., 37 degrees centigrade, to speed evaporation.

The kit further includes a buffered saline solution, e.g., phosphate buffered saline. The surface of the user's face is moistened with the buffered saline and the microbe sampling mask applied to the skin surface. After about 10 minutes, the microbe sampling mask is removed from the skin surface and allowed to dry.

The analyzer of the kit includes a receptacle sized to fit the microbe sampling mask and a conveyor system for moving the microbe sampling mask within the analyzer. Once in the analyzer, the microbe sampling mask is moved from a first reservoir containing fluorescence-labeled antibodies, to a second reservoir containing a wash solution, and into proximity of a sensor component. The fluorescence-labeled antibodies in the first reservoir include an antibody directed against Staphylococcus epidermidis and an antibody directed against Staphylococcus aureus (e.g., from Thermo Scientific Pierce, Rockford, Ill.) labeled with fluorescein and rhodamine, respectively, using standard labeling kits (e.g., from Molecular Probes/Invitrogen, Carlsbad, Calif.). The wash solution in the second reservoir includes phosphate buffered saline and 1% Tween20.

The analyzer includes electromagnetic energy emitters, e.g., a krypton and/or argon laser, for exciting the fluorescein and/or rhodamine fluorophores associated with the fluorescence-labeled antibodies and one or more CMOS sensors to detect emitted fluorescence. For example, fluorescein has an excitation wavelength of about 480 nm and a peak emission wavelength of about 520 nm. For example, rhodamine has an excitation wavelength of about 510 nm and a peak emission wavelength of about 560 nm. The signals emitted from the surface of the mask are transformed into a sensor output.

The analyzer includes a computing component to receive the sensor output from the one or more CMOS sensors. The sensor output includes properties of signals, e.g., signals at 520 nm (green fluorescence) and 560 nm (red fluorescence), that are compared with a dataset of reference signal properties. The distribution and intensity of green and red fluorescence are incorporated into a microbe profile of the user. A series of relative ratios of the green and red fluorescence is used to estimate the relative distribution and abundance of Staphylococcus epidermidis versus Staphylococcus aureus over the skin surface of the user. The distribution of relative ratios is compared with a normal distribution of relative ratios. The computing component determines that the relative ratios for the user are outside the norm in some locations, e.g., the forehead and nose, but within the norm in other locations, e.g., the cheeks and chin, on the user's face. The computing component recommends selective use of the probiotic including Staphylococcus epidermidis on the user's forehead and nose. The computing component also recommends one of the three cosmetic formulations depending upon the skin characteristics of the user, e.g., oily, normal, or dry. The computing component further recommends rescreening of the face after two weeks of treatment to determine whether the relative distribution and abundance of Staphylococcus epidermidis versus Staphylococcus aureus over the skin surface of the user has shifted closer to the norm. Once the updated analysis has been completed, the computing component updates the recommended treatment agents to reflect any changes in the relative ratios. For rescreening, the pre-formed mask is placed in a residential dishwasher to remove bound bacteria and antibodies. After cleaning, the pre-formed mask is retreated with poly-L-lysine and is ready for microbe sampling.

### Prophetic Example 3

### A Kit Including Treatment Agents, Peelable Skin-Covering Material, and Analyzer for Assessment and Treatment of Candida albicans

A kit is described that includes treatment agents, a peelable skin-covering material, and analyzer for use in accessing Candida albicans on a skin surface of a user being treated with antibiotics. High doses of antibiotics can lead to reduced levels of healthy bacteria on skin allowing for increased fungal growth.

The kit includes treatment agents in individual containers. The treatment agents include nystatin fungicide in three forms, nystatin ointment, nystatin cream, and nystatin topical powder, and two probiotics including Lactobacillus and Staphylococcus epidermidis. Nystatin cream is preferred in intertriginous areas, e.g., skin folds, while nystatin powder is preferred in very moist regions, e.g., between toes or under arms.

The kit includes a settable material, e.g., gelatin, and a polyclonal antibody that recognizes and is able to capture Candida albicans (from, e.g., Accurate Chemical & Scientific Co., Westbury, N.Y.). The gelatin is prepared and applied to a skin surface of the user. Briefly, 7 grams of gelatin, e.g., 1 0.25 ounce packet of non-flavored Knox® Gelatine (from Kraft Foods, Northfield, Ill.) is heated in the presence of 125 milliliters of water to completely dissolve the gelatin. The gelatin solution is allowed to cool for 20 minutes at 4 degrees centigrade. The polyclonal antibody active against Candida albicans is added to the cooled gelatin. The gelatin solution including the polyclonal antibody is applied as a thin patch to the surface of the users's skin and allowed to air dry for 15 to 30 minutes.

The gelatin patch is gently removed from the skin surface. The inner surface of the gelatin patch carrying the antibody-bound microbes is placed on the scanning surface of the analyzer and imaged with directed electromagnetic energy at wavelengths of 465-495 nm to induce Candida albicans autofluoresces at an emission wavelength of 515-555 mm (see, e.g., Mateus et al. (2004) Antimicrob. Agents Chemother. 48:3358-3336, which is incorporated herein by reference). The autofluorescence is captured using an image-capture device, e.g., a CMOS sensor, and the resulting signal data is transformed into a sensor output for receipt by a computing component of the analyzer.

Alternatively, a second antibody directed against Candida albicans and labeled with a fluorescent dye can be used to detect the fungus captured on the antibodies in the gelatin patch. For example, the gelatin patch can be exposed to a polyclonal antibody directed against Candida albicans and conjugated to fluorescein (FITC, from, e.g., Thermo Scientific Pierce Antibodies, Rockford, Ill.). The labeled antibody is supplied as part of the kit, either as a separate container or incorporated into a reservoir of the analyzer. The labeled antibody is applied to the surface of the gelatin patch for 30 minutes. Unbound antibody is removed from the gelatin patch with a phosphate buffered saline/1% Tween wash solution. After a rinse, the gelatin patch is inserted into the analyzer and subjected to directed energy, e.g., an excitation wavelength of about 480 nm to cause the fluorescein associated with the antibody to fluoresce at about 520 nm. The resulting fluorescence signal is captured with the CMOS sensor and transformed into a digital output.

Alternatively, a fluorescent stain, e.g., Calcofluor White Stain, that binds cellulose and chitin in fungal cell walls, can be used to detect Candida albicans on the gelatin patch. The Calcofluor White Stain is supplied as part of the kit, either as a separate container or incorporated into a reservoir of the analyzer. The gelatin patch is treated with equal part Calcofluor White Stain (from, e.g., Sigma Aldrich, St. Louis, Mo.) and 10% potassium hydroxide. After rinsing with buffered saline, the gelatin patch is inserted into the analyzer and subjected to an excitation wavelength of about 350 nm and a blue light emission at about 400 nm captured with the CMOS sensor.

The computing component of the analyzer receives the sensor output from the CMOS sensor and compares the properties of the signals emitted from the surface of the gelatin patch with a dataset of reference signal properties to generate a microbe profile including the spatial distribution of Candida albicans on the gelatin patch. The intensity of the signals received from any given spot on the gelatin patch may also be used to estimate the relative abundance of the fungus. The computing component generates a microbe profile for the user and compares the microbe profile with a “normalized” reference microbe profile for the skin region sampled, e.g., face versus armpit versus foot. The computing component notes that the relative abundance of Candida albicans in the face of the user is above normal and recommends to the user use of the ointment form of nystatin as well as the probiotic including Staphylococcus epidermidis to add back normal, healthy bacteria to repopulate the skin surface. The computing component also provides the user a recommended date in the future, e.g., 2-3 weeks in the future, for a repeat analysis of the skin surface.

### Prophetic Example 4

### A Kiosk for Dispensing One or More Treatment Agents Based on a Microbe Profile

A kiosk is described for dispensing microbe sampling swabs, analyzing the swabs, selecting treatment agents, and dispensing the treatment agents. The kiosk includes a store of small bottles containing one or more treatment agents, and a store of microbe sampling swabs, as well as receiving and dispensing components, a user interface, a sensor component, and a computing component.

The kiosk includes a store of small bottles. Each small bottle contains a probiotic solution, e.g., including Lactobacillus and/or Staphylococcus epidermidis, a cosmetic foundation, e.g., in one or more color tones, a prebiotic solution, e.g., oligosaccharides, or a combination thereof. Each bottle contains a label with product information, use instructions, and a bar code for use by the kiosk to identify the contents of the bottle.

The kiosk further includes a store of microbe sampling swabs, e.g., cotton swabs. Each microbe sampling swab is individually wrapped to prevent contamination prior to sampling a body surface.

The kiosk includes a touchscreen display user interface. The touchscreen is activated by initial touch by the user. The user uses the touchscreen display to read instructions regarding the use of the kiosk. The user also uses the touchscreen display to enter user information including age, gender, ethnicity, skin characteristics, medical history, co-morbidities, and user preferences. It may also be necessary for the user to enter an identification code, e.g., a pin number, associated with the user's account. The touchscreen display is also used in combination with a bill or charge card acceptor for billing purposes.

The kiosk dispenses one or more microbe sampling swabs to the user. The user is instructed to use the swabs to sample one or more body surfaces. For example, the user can be instructed to swab a skin or mouth surface while standing at the kiosk. For example, the user may be instructed to go to a more private location, e.g., a restroom, to sample a vaginal surface. Once the user has completed sampling the body surfaces, the user uses the touchscreen display to enter the location of each body surface sampled and inserts each microbe sampling swab into a receiving component, e.g., a compartment with a hinged door, of the kiosk.

The microbe sampling swab is processed by soaking the swab in a solution of buffered saline to dislodge attached microbes. To the solution is added fluorescence-labeled antibodies directed against Staphylococcus epidermidis (fluorescein label) Staphylococcus aureus (phycoerythrin label) and Candida albicans (Cy7 label) for use in multi-color flow cytometry.

The kiosk further includes components of a flow cytometer including a flow cell, a laser excitation energy source, lens and filters, and fluorescence detectors, e.g., photomultiplier tubes, photodiodes, or CMOS detectors, as well as electronics to amplify and process the detected signals. As the solution including the microbes from the microbe sampling swab and the fluorescence-labeled antibodies pass through the flow cell, particles are subjected to excitation energy causing any fluorophores present to fluoresce. The different fluorophores have peak emission wavelengths that can be delineated using bandpass filters. The detected signals are transformed into a sensor output.

The computing component receives the sensor output, including properties of the fluorescing events. The three fluorescent parameters representative of Staphylococcus epidermidis (fluorescein) Staphylococcus aureus (phycoerythrin) and Candida albicans (Cy7) are plotted on a linear or log scale to generate a microbe profile for the user. The computing component compares the microbe profile for the user against “normalized” age, gender, or skin type matched reference microbe profiles. The computing component selects one or more of the available probiotics, prebiotics, and/or cosmetic foundations for the user based on the comparison. For example, the detection of high levels of Staphylococcus aureus and/or Candida albicans (Cy7) relative to Staphylococcus epidermidis levels would lead to a recommended use of a probiotic containing Staphylococcus epidermidis.

The computing component of the kiosk sends a signal to the dispensing component of the kiosk to dispense the bottles of recommended treatment agents. In addition, the computing component also provides to the user a printout describing use of the recommended treatment agents as well as additional treatment options, e.g., cleansing, dietary, or other medication options. The computing component may further dispense one or more coupons related to these additional treatment options, e.g., a coupon for 50% off a specific cleansing product appropriate for the user's microbe profile.

### Prophetic Example 5

### A Method for Selecting and Arranging for Delivery of One or More Treatment Agents

A method is described for selecting and arranging for delivery of one or more treatment agents in response to receiving a microbe profile from an individual. The method is implemented on a computer system associated with a retailer, e.g., a skincare product retailer. For example, the computer system of the skincare product retailer can include software for implementing the method for selecting and arranging for delivery of the one or more treatment agents in response to receiving the microbe profile from the individual.

The computer system of the skincare product retailer periodically, e.g., every month, receives a microbe profile from an individual as part of a skincare product purchase program. The skincare product purchase program is a fee-based program in which an individual periodically receives one or more specific treatment agents from the skincare product retailer based on the individual's specific microbe profile. The skincare product purchase program further includes automatically receiving a resupply of at least one microbe sampling unit in response to the skincare product retailer receiving a recent microbe profile from the individual. The microbe profile from the individual is received via e-mail through an Internet connection from the computing component of an at-home microbe profiling kit. The microbe profile includes the distribution of one or more types of microbes on a skin surface of the individual.

The computer system of the skincare product retailer selects one or more treatment agents from a list of available agents to modulate at least one of the one or more types of microbes in the microbe profile of the individual. The computer system includes a dataset with a listing of all of the treatment agents available to the skincare product retailer, either in house or from another source. The dataset also includes a list of microbes matched with appropriate treatment agents. The computer system notes the microbes represented in the received microbe profile, looks for the microbes in the dataset, and selects the matched treatment agents. The computer system also notes specifics of user information, e.g., medical history or co-morbidities, and excludes any of the selected matched treatment agents for which contraindications exist, e.g., pregnancy, diabetes, or other medical condition.

The computer system of the skincare product retailer determines whether or not the selected one or more treatment agents are in stock. If the selected treatment agents are in stock, the computing system of skincare product retailer sends a message to the warehouse including instructions for packaging the selected treatment agents and labeling the package for shipment. The computer system of the skincare product retailer further contacts a shipping company, e.g., FedEx, and arranges for pickup and delivery of the package containing the treatment agents to the user. The computer system of the skincare product retailer notifies the user via e-mail that the package has been picked up and provides an estimated date of delivery.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A kit, comprising:
a plurality of treatment agents;
a microbe sampling unit having at least one surface with a microbe-capture region, the microbe-capture region configured to capture one or more types of microbes from a body surface of a user; and
an analyzer including
at least one sensor component including circuitry configured to detect one or more signals emitted or reflected from the microbe-capture region of the microbe sampling unit and to transform the detected one or more signals into a sensor output;
a user interface; and
a computing component including a processor and operably coupled to the at least one sensor component and the user interface, the computing component including circuitry configured to
receive the sensor output from the at least one sensor component, the sensor output including information associated with at least one property of the detected one or more signals emitted or reflected from the microbe-capture region of the microbe sampling unit;
compare the at least one property of the detected one or more signals emitted or reflected from the microbe-capture region of the microbe sampling unit with a reference dataset of signal properties;
generate a microbe profile of the user based on the comparison with the reference dataset of signal properties;
compare the microbe profile of the user with at least one reference microbe profile; and
recommend to the user at least one of the plurality of treatment agents based on the comparison with the at least one reference microbe profile.

2. The kit of claim 1, wherein the plurality of treatment agents comprises at least one first treatment agent in at least one first container and at least one second treatment agent in at least one second container.

3. The kit of claim 2, wherein the plurality of treatment agents comprises at least one third treatment agent in at least one third container.

4. The kit of claim 1, wherein the plurality of treatment agents comprises one or more probiotic agents.

5. The kit of claim 4, wherein the one or more probiotic agents comprise at least one type of bacteria from Firmicutes, Actinobacteria, Bacteriodetes, Proteobacteria, or Cyanobacteria.

6. The kit of claim 4, wherein the one or more probiotic agents comprise at least one type of bacteria from Corynebacteria, Propionibacteria, Micrococci, or Staphylococci.

7. The kit of claim 4, wherein the one or more probiotic agents comprise at least one of a non-pathogenic strain of a pathogenic bacterium.

8. The kit of claim 4, wherein the one or more probiotic agents comprise Staphylococcus epidermidis.

9. The kit of claim 1, wherein the plurality of treatment agents comprise one or more prebiotic agents.

10. The kit of claim 9, wherein the one or more prebiotic agents comprise at least one of oligosaccharides, inulin, or lactulose.

11. The kit of claim 1, wherein the plurality of treatment agents comprise one or more antimicrobial agents.

12. The kit of claim 11, wherein the one or more antimicrobial agents comprise at least one of an antibacterial agent, an antifungal agent, or an antiviral agent.

13. The kit of claim 1, wherein the plurality of treatment agents comprise one or more therapeutic agents.

14. The kit of claim 13, wherein the one or more therapeutic agents comprise at least one of an anti-inflammatory agent, a chemotherapeutic agent, an antiseptic agent, an anesthetic agent, or an anti-acne agent.

15. The kit of claim 1, wherein the plurality of treatment agents comprise at least one of a moisturizer, an astringent, an anti-aging treatment agent, a retinoid agent, or a cosmetic agent.

16. The kit of claim 1, wherein the microbe-capture region of the microbe sampling unit is configured to capture one or more types of microbes from a skin surface of the user.

17. The kit of claim 1, wherein the microbe-capture region of the microbe sampling unit is configured to capture one or more types of microbes from a gastrointestinal surface of the user.

18. The kit of claim 1, wherein the microbe-capture region of the microbe sampling unit is configured to capture one or more types of microbes from a mucosal surface, a vaginal surface, a nasal surface, or a mouth surface of the user.

19. The kit of claim 1, wherein the microbe sampling unit comprises a mask.

20. The kit of claim 19, wherein the mask comprises a pre-formed mask.

21. The kit of claim 19, wherein the mask comprises a peelable mask.

22. The kit of claim 1, wherein the microbe sampling unit comprises a mouthpiece.

23. The kit of claim 1, wherein the microbe sampling unit comprises a strip.

24. The kit of claim 1, wherein the microbe sampling unit includes at least one optical sensor.

25. The kit of claim 1, wherein the microbe sampling unit substantially conforms to a topography of the body surface of the user.

26. The kit of claim 1, wherein the microbe sampling unit comprises a swab.

27. The kit of claim 1, wherein the microbe sampling unit comprises a brush.

28. The kit of claim 1, wherein the microbe sampling unit comprises a razor.

29. The kit of claim 1, wherein the microbe sampling unit is personalized for the user.

30. The kit of claim 1, wherein the microbe sampling unit and the analyzer are incorporated into a hand-held microbe profiling device.

31. The kit of claim 30, wherein the hand-held microbe profiling device includes a brush head.

32. The kit of claim 30, wherein the hand-held microbe profiling device includes a razor head.

33. The kit of claim 30, wherein the hand-held microbe profiling device includes a rotatable microbe sampling unit including the microbe-capture region.

34. The kit of claim 30, wherein the hand-held microbe profiling device includes a vacuum.

35. The kit of claim 1, wherein the microbe-capture region of the microbe sampling unit comprises a charged surface.

36. The kit of claim 1, wherein the microbe-capture region of the microbe sampling unit comprises at least one of an adhesive, an absorbent, or an adsorbent.

37. The kit of claim 1, wherein the microbe-capture region of the microbe sampling unit comprises a biomolecule-binding polymer.

38. The kit of claim 1, wherein the microbe-capture region of the microbe sampling unit comprises a gel.

39. The kit of claim 1, wherein the microbe-capture region of the microbe sampling unit comprises a plurality of specific microbe-binding elements.

40. The kit of claim 39, wherein at least one of the plurality of specific microbe-binding elements comprises a specific microbe-binding antibody.

41. The kit of claim 39, wherein at least one of the plurality of specific microbe-binding elements comprises a specific microbe-binding oligonucleotide.

42. The kit of claim 39, wherein at least one of the plurality of specific microbe-binding elements comprises a protein, a peptide, DNA, RNA, a lectin, a carbohydrate, an anti-16S rRNA ligand, an aptamer, a synthetic ligand, or a mimetic binding element.

43. The kit of claim 39, wherein the microbe-capture region comprises a plurality of specific microbe-binding elements of at least one first type and a plurality of specific microbe-binding elements of at least one second type.

44. The kit of claim 1, wherein the microbe-capture region of the microbe sampling unit comprises a plurality of signal-generating complexes.

45. The kit of claim 44, wherein at least one of the plurality of signal-generating complexes comprises an optical signal-generating complex, a fluorescing signal-generating complex, electromagnetic signal-generating complex, a radio signal-generating complex, an electrical current signal-generating complex, an acoustic signal-generating complex, or a magnetic signal-generating complex.

46. The kit of claim 44, wherein the microbe-capture region comprises a plurality of signal-generating complexes of at least one first type able to emit at least one first signal type in response to at least one first type of microbe and a plurality of signal-generating complexes of at least one second type able to emit at least one second signal type in response to at least one second type of microbe.

47. The kit of claim 44, wherein each of the plurality of signal-generating complexes comprises at least one signal-generating element operably coupled to at least one specific microbe-binding element, the at least one signal-generating element configured to emit one or more signals in response to contact with at least one type of microbe by the operably coupled at least one specific microbe-binding element.

48. The kit of claim 1, wherein the analyzer includes a receptacle sized to fit at least a portion of the microbe sampling unit.

49. The kit of claim 1, wherein the at least one sensor component of the analyzer includes at least one optical sensor.

50. The kit of claim 1, wherein the at least one sensor component of the analyzer includes at least one fluorescence sensor.

51. The kit of claim 1, wherein the at least one sensor component of the analyzer includes at least one of an electromagnetic sensor component, an electrical current sensor component, a piezoelectric sensor component, a magnetic sensor component, an acoustic sensor component, a radiofrequency sensor component, or a radioactivity sensor component.

52. The kit of claim 1, wherein the at least one sensor component of the analyzer includes circuitry configured to detect one or more signals emitted or reflected from at least one of the one or more types of microbes captured on the microbe-capture region of the microbe sampling unit.

53. The kit of claim 1, wherein the at least one sensor component of the analyzer includes circuitry configured to detect one or more signals emitted or reflected from at least one of a plurality of signal-generating elements associated with the one or more types of microbes captured on the microbe-capture region of the microbe sampling unit.

54. The kit of claim 1, wherein the at least one sensor component of the analyzer includes circuitry configured to detect one or more signals emitted from at least one of a plurality of signal-generating complexes associated with the microbe sampling unit in response to contact with at least one of the one or more types of microbes.

55. The kit of claim 1, wherein the user interface of the analyzer includes at least one of a display, touchscreen, keyboard, microphone, speaker, mouse, joystick, buttons, switches, or printer.

56. The kit of claim 1, wherein the analyzer further includes one or more reservoirs.

57. The kit of claim 56, wherein at least one of the one or more reservoirs includes a plurality of at least one type of signal-generating element.

58. The kit of claim 56, wherein at least one of the one or more reservoirs includes at least one of a buffer, a detergent solution, a reagent solution, or a wash solution.

59. The kit of claim 1, wherein the at least one property of the detected one or more signals emitted or reflected from the microbe-capture region of the microbe sampling unit comprises at least one of an optical property, a fluorescent property, a magnetic property, an electrical property, an electromagnetic property, an acoustic property, or a radiofrequency property.

60. The kit of claim 1, wherein the reference dataset of signal properties includes properties of one or more signals emitted or reflected from a given type of microbe.

61. The kit of claim 1, wherein the reference dataset of signal properties includes properties of one or more signals emitted from a given type of signal-generating element.

62. The kit of claim 1, wherein the reference dataset of signal properties includes properties of one or more signals emitted from a given type of signal-generating complex.

63. The kit of claim 1, wherein the reference dataset of signal properties is included in a memory component of the computing component of the analyzer.

64. The kit of claim 1, wherein the reference dataset of signal properties is accessed from a remote computing device through a communication link.

65. The kit of claim 1, wherein the at least one reference microbe profile includes at least one historical microbe profile of the user.

66. The kit of claim 1, wherein the at least one reference microbe profile includes at least one microbe profile from one or more other individuals.

67. The kit of claim 1, wherein the at least one reference microbe profile includes at least one theoretical microbe profile.

68. The kit of claim 1, wherein the at least one reference microbe profile is included in a memory component of the computing component of the analyzer.

69. The kit of claim 1, wherein the at least one reference microbe profile is accessed from a remote computing device through a communication link.

70. The kit of claim 1, wherein the analyzer further includes a transmission unit including an antenna.

71. The kit of claim 1, wherein the computing component of the analyzer includes circuitry configured to recommend to the user at least one of the plurality of treatment agents based on an identity of one or more types of microbes in the microbe profile.

72. The kit of claim 1, wherein the computing component of the analyzer includes circuitry configured to recommend to the user at least one of the plurality of treatment agents based on an identity of one or more types of microbes in the microbe profile and one or more other factors.

73. The kit of claim 72, wherein the one or more other factors include one or more of age, gender, ethnicity, skin characteristic, geographical location, medical history, co-morbidities, or user preference.

74. The kit of claim 72, wherein the one or more other factors include at least one user preference.

75. The kit of claim 1, wherein the computing component of the analyzer includes circuitry to receive user information from the user through the user interface.

76. The kit of claim 75, wherein the user information includes at least one of age, gender, ethnicity, skin characteristic, geographical location, medical history, co-morbidities, or user preference.

77. The kit of claim 1, wherein the computing component of the analyzer includes circuitry configured to report at least one of the microbe profile, the recommended at least one of the plurality of treatment agents, user information, or other information to one or more of a medical record, a healthcare provider, a pharmacy, a cosmetologist, a merchant, a supplier, or a manufacturer.

78. The kit of claim 1, wherein the computing component of the analyzer includes circuitry configured to report at least one of the microbe profile, the recommended at least one of the plurality of treatment agents, user information, or other information to at least one of a website, a social media site, or a personal computing device.

79. The kit of claim 1, wherein the computing component of the analyzer includes circuitry configured to automatically call out to at least one of a healthcare provider, a pharmacy, a cosmetologist, a merchant, a supplier, or a manufacturer to request resupply of at least one of the plurality of treatment agents.

80. The kit of claim 1, wherein the computing component of the analyzer includes circuitry configured to
store the microbe profile in a memory component of the computing component;
chart changes in the microbe profile over time as the analyzer generates one or more additional microbe profiles of the user; and
report the charted changes in the microbe profile to the user.

81. The kit of claim 1, further including a set of user instructions for using the kit.

82. The kit of claim 81, wherein the set of user instructions is on non-transitory machine readable media.

83. The kit of claim 81, wherein the set of instructions includes at least one of one or more instructions for sampling the body surface of the user with the microbe sampling unit, one or more instructions for using the analyzer, one or more instructions for interpreting the microbe profile, one or more instructions for applying the recommended at least one of the plurality of treatment agents to the body surface, or one or more instructions for accessing a website.

84. A kiosk, comprising:
a plurality of treatment agents;
one or more dispensers to dispense at least one of the plurality of treatment agents;
at least one microbe sampling unit, the at least one microbe sampling unit including at least one surface with a microbe-capture region, the microbe-capture region configured to capture one or more types of microbes from a body surface of a user;
a user interface;
at least one sensor component including circuitry configured to detect one or more signals emitted or reflected from the microbe-capture region of the at least one microbe sampling unit and to transform the detected one or more signals into a signal output; and
a computing component including a processor and operably coupled to the one or more dispensers, the at least one sensor component, and the user interface, the computing component including circuitry configured to
receive the sensor output from the at least one sensor component, the sensor output including information associated with at least one property of the detected one or more signals emitted or reflected from the microbe-capture region of the at least one microbe sampling unit;
compare the at least one property of the detected one or more signals emitted or reflected from the microbe-capture region of the at least one microbe sampling unit with a reference dataset of signal properties;
generate a microbe profile of the user based on the comparison with the reference dataset of signal properties;
compare the microbe profile of the user with at least one reference microbe profile;
recommend at least one of the plurality of treatment agents to the user based on the comparison with the at least one reference microbe profile; and
send a signal to at least one of the one or more dispensers to dispense the recommended at least one of the plurality of treatment agents from the kiosk to the user.

85. The kiosk of claim 84, further including a microbe sampling unit dispensing component operably coupled to the computing component, the microbe sampling unit dispensing component to dispense the at least one microbe sampling unit to the user.

86. The kiosk of claim 84, further including at least one receiving component operably coupled to the computing component, the receiving component to receive the at least one microbe sampling unit from the user.

87. The kiosk of claim 86, wherein the at least one receiving component includes a feeding mechanism.

88. The kiosk of claim 84, wherein the plurality of treatment agents comprise at least one first treatment agent in at least one first container and at least one second treatment agent in at least one second container.

89. The kiosk of claim 88, wherein the plurality of treatment agents comprises at least one third treatment agent in at least one third container.

90. The kiosk of claim 84, wherein the plurality of treatment agents comprise at least one first treatment agent dispensable by at least one first dispenser and at least one second treatment agent dispensable by at least one of the at least one first dispenser or at least one second dispenser.

91. The kiosk of claim 84, wherein the plurality of treatment agents comprises one or more probiotic agents.

92. The kiosk of claim 91, wherein the one or more probiotic agents comprise at least one type of bacteria from Firmicutes, Actinobacteria, Bacteriodetes, Proteobacteria, or Cyanobacteria.

93. The kiosk of claim 91, wherein the one or more probiotic agents comprise at least one type of bacteria from Corynebacteria, Propionibacteria, Micrococci, or Staphylococci.

94. The kiosk of claim 91, wherein the one or more probiotic agents comprise at least one of a non-pathogenic strain of a pathogenic bacterium.

95. The kiosk of claim 91, wherein the one or more probiotics comprise Staphylococcus epidermidis.

96. The kiosk of claim 84, wherein the plurality of treatment agents comprise one or more prebiotic agents.

97. The kiosk of claim 96, wherein the one or more prebiotic agents comprise at least one of oligosaccharides, inulin, or lactulose.

98. The kiosk of claim 84, wherein the plurality of treatment agents comprise one or more antimicrobial agents.

99. The kiosk of claim 98, wherein the one or more antimicrobial agents comprise at least one of an antibacterial agent, an antifungal agent, or an antiviral agent.

100. The kiosk of claim 84, wherein the plurality of treatment agents comprise one or more therapeutic agents.

101. The kiosk of claim 100, wherein the one or more therapeutic agents comprise at least one of an anti-inflammatory agent, a chemotherapeutic agent, an antiseptic agent, an anesthetic agent, or an anti-acne agent.

102. The kiosk of claim 84, wherein the plurality of treatment agents comprise at least one of a moisturizer, an astringent, an anti-aging treatment agent, a retinoid agent, or a cosmetic agent.

103. The kiosk of claim 84, wherein the microbe-capture region of the at least one microbe sampling unit is configured to capture one or more types of microbes from a skin surface of the user.

104. The kiosk of claim 84, wherein the microbe-capture region of the at least one microbe sampling unit is configured to capture one or more types of microbes from a gastrointestinal surface of the user.

105. The kiosk of claim 84, wherein the microbe-capture region of the at least one microbe sampling unit is configured to capture one or more types of microbes from a mucosal surface, a vaginal surface, a nasal surface, or a mouth surface of the user.

106. The kiosk of claim 84, wherein the at least one microbe sampling unit comprises a mask.

107. The kiosk of claim 106, wherein the mask comprises a preformed mask.

108. The kiosk of claim 106, wherein the mask comprises a peelable mask.

109. The kiosk of claim 84, wherein the at least one microbe sampling unit comprises a mouthpiece.

110. The kiosk of claim 84, wherein the at least one microbe sampling unit comprises a strip.

111. The kiosk of claim 84, wherein the at least one microbe sampling unit substantially conforms to a topography of the body surface of the user.

112. The kiosk of claim 84, wherein the at least one microbe sampling unit comprises an optical sensor.

113. The kiosk of claim 84, wherein the at least one microbe sampling unit comprises a swab.

114. The kiosk of claim 84, wherein the at least one microbe sampling unit comprises a brush.

115. The kiosk of claim 84, wherein the at least one microbe sampling unit comprises a razor.

116. The kiosk of claim 84, wherein the at least one microbe sampling unit includes a rotatable microbe-capture region.

117. The kiosk of claim 84, wherein the at least one microbe sampling unit is personalized for the user.

118. The kiosk of claim 84, wherein the microbe-capture region of the at least one microbe sampling unit comprises a charged surface.

119. The kiosk of claim 84, wherein the microbe-capture region of the at least one microbe sampling unit comprises at least one of an adhesive, an absorbent, or an adsorbent.

120. The kiosk of claim 84, wherein the microbe-capture region of the at least one microbe sampling unit comprises a biomolecule-binding polymer.

121. The kiosk of claim 84, wherein the microbe-capture region of the at least one microbe sampling unit comprises a gel.

122. The kiosk of claim 84, wherein the microbe-capture region of the microbe sampling unit comprises a plurality of specific microbe-binding elements.

123. The kiosk of claim 122, wherein at least one of the plurality of specific microbe-binding elements comprises a specific microbe-binding antibody.

124. The kiosk of claim 122, wherein at least one of the plurality of specific microbe-binding elements comprises a specific microbe-binding oligonucleotide.

125. The kiosk of claim 122, wherein at least one of the plurality of specific microbe-binding elements comprises a protein, a peptide, a lectin, a carbohydrate, RNA, DNA, an anti-16S rRNA ligand, an aptamer, a synthetic ligand, or a mimetic binding element.

126. The kiosk of claim 122, wherein the microbe-capture region comprises a plurality of specific microbe-binding elements of at least one first type and a plurality of specific microbe-binding elements of at least one second type.

127. The kiosk of claim 84, wherein the microbe capture region of the at least one microbe sampling unit comprises a plurality of signal-generating complexes.

128. The kiosk of claim 127, wherein at least one of the plurality of signal-generating complexes comprises an optical signal-generating complex, a fluorescing signal-generating complex, electromagnetic signal-generating complex, a radio signal-generating complex, an electrical current signal-generating complex, an acoustic signal-generating complex, or a magnetic signal-generating complex.

129. The kiosk of claim 127, wherein each of the plurality of signal-generating complexes comprises at least one signal-generating element operably coupled to at least one specific microbe-binding element, the at least one signal-generating element configured to emit one or more signals in response to contact with at least one type of microbe by the operably coupled at least one specific microbe-binding element.

130. The kiosk of claim 127, wherein the microbe-capture region comprises a plurality of signal-generating complexes of at least one first type able to emit at least one first signal type in response to at least one first type of microbe and a plurality of signal-generating complexes of at least one second type able to emit at least one second signal type in response to at least one second type of microbe.

131. The kiosk of claim 84, wherein the at least one sensor component includes at least one optical sensor.

132. The kiosk of claim 84, wherein the at least one sensor component includes at least one fluorescence sensor.

133. The kiosk of claim 84, wherein the at least one sensor component includes at least one of an electromagnetic sensor component, an electrical current sensor component, a piezoelectric sensor component, a magnetic sensor component, an acoustic sensor component, a radiofrequency sensor component, or a radioactivity sensor component.

134. The kiosk of claim 84, wherein the at least one sensor component includes circuitry configured to detect one or more signals emitted or reflected from at least one of the one or more types of microbes captured on the microbe-capture region of the at least one microbe sampling unit.

135. The kiosk of claim 84, wherein the at least one sensor component includes circuitry configured to detect one or more signals emitted or reflected from at least one of a plurality of signal-generating elements associated with the one or more types of microbes captured on the microbe-capture region of the at least one microbe sampling unit.

136. The kiosk of claim 84, wherein the at least one sensor component includes circuitry configured to detect one or more signals emitted from at least one of a plurality of signal-generating complexes associated with the at least one microbe sampling unit in response to contact with at least one of the one or more types of microbes.

137. The kiosk of claim 84, wherein the user interface includes at least one of a display, touchscreen, keyboard, microphone, speaker, mouse, joystick, buttons, switches, or printer.

138. The kiosk of claim 84, wherein the kiosk further includes one or more reservoirs.

139. The kiosk of claim 138, wherein at least one of the one or more reservoirs includes a plurality of at least one type of signal-generating element.

140. The kiosk of claim 138, wherein at least one of the one or more reservoirs includes at least one of a buffer, a detergent solution, a reagent solution, or a wash solution.

141. The kiosk of claim 84, wherein the at least one property of the detected one or more signals emitted or reflected from the microbe-capture region of the microbe sampling unit comprises at least one of an optical property, a fluorescent property, a magnetic property, an electrical property, an electromagnetic property, or a radiofrequency property.

142. The kiosk of claim 84, wherein the reference dataset of signal properties includes properties of one or more signals emitted or reflected from a given type of microbe.

143. The kiosk of claim 84, wherein the reference dataset of signal properties includes properties of one or more signals emitted from a given type of signal-generating element.

144. The kiosk of claim 84, wherein the reference dataset of signal properties includes properties of one or more signals emitted from a given type of signal-generating complex.

145. The kiosk of claim 84, wherein the reference dataset of signal properties is included in a memory component of the computing component.

146. The kiosk of claim 84, wherein the reference dataset of signal properties is accessed from a remote computing device through a communication link.

147. The kiosk of claim 84, wherein the at least one reference microbe profile comprises at least one historical microbe profile of the user.

148. The kiosk of claim 84, wherein the at least one reference microbe profile comprises at least one microbe profile from one or more other individuals.

149. The kiosk of claim 84, wherein the at least one reference microbe profile comprises at least one theoretical microbe profile.

150. The kiosk of claim 84, wherein the at least one reference microbe profile is included in a memory component of the computing component.

151. The kiosk of claim 84, wherein the at least one reference microbe profile is accessed from a remote computing device through a communication link.

152. The kiosk of claim 84, further including a transmission unit including an antenna.

153. The kiosk of claim 84, wherein the computing component includes circuitry configured to recommend to the user at least one of the plurality of treatment agents based on an identity of one or more types of microbes in the microbe profile of the user.

154. The kiosk of claim 84, wherein the computing component includes circuitry configured to recommend to the user at least one of the plurality of treatment agents based on an identity of one or more types of microbes included in the microbe profile of the user and one or more other factors.

155. The kiosk of claim 154, wherein the one or more other factors include one or more of age, gender, ethnicity, skin characteristic, geographical location, medical history, or co-morbidities.

156. The kiosk of claim 154, wherein the one or more other factors include at least one user preference.

157. The kiosk of claim 84, wherein the computing component includes circuitry configured to receive user information from the user through the user interface.

158. The kiosk of claim 157, wherein the user information includes at least one of age, gender, ethnicity, skin characteristics, geographical location, medical history, co-morbidities, or user preferences.

159. The kiosk of claim 84, wherein the computing component includes circuitry configured to report at least one of the microbe profile, the recommended at least one of the plurality of treatment agents, user information, or other information to one or more of a medical record, a healthcare provider, a pharmacy, a cosmetologist, a merchant, a supplier, or a manufacturer.

160. The kiosk of claim 84, wherein the computing component includes circuitry configured to report at least one of the microbe profile, the recommended at least one of the plurality of treatment agents, user information, or other information to at least one of a website, a social media site, or a personal computing device.

161. The kiosk of claim 84, wherein the computing component includes circuitry configured to automatically call out to at least one of a healthcare provider, a pharmacy, a cosmetologist, a merchant, a supplier, or a manufacturer to request resupply of at least one of the plurality of treatment agents.

162. The kiosk of claim 84, wherein the computing component includes circuitry configured to receive one or more user identifiers.

163. The kiosk of claim 84, wherein the computing component includes circuitry configured to attach one or more user identifiers to the microbe profile of the user.

164. The kiosk of claim 84, wherein the computing component includes circuitry configured to
store the microbe profile of the user in a memory component of the computing component;
chart changes in the microbe profile over time as the computing component generates one or more additional microbe profiles of the user; and
report the charted changes in the microbe profile to the user.

165. The kiosk of claim 84, further including a set of user instructions for using the kiosk or a component of the kiosk.

166. The kiosk of claim 165, wherein the set of user instructions includes at least one of one or more instructions for using the kiosk, one or more instructions for sampling the body surface of the user with the at least one microbe sampling unit, one or more instructions for inserting the at least one microbe sampling unit into at least one receiving component of the kiosk, one or more instructions for interpreting the microbe profile, one or more instructions for applying the recommended at least one of the plurality of treatment agents to the body surface, or one or more instructions for accessing a website.

167. A method, comprising:
receiving information associated with a microbe profile of an individual from a remote source, the microbe profile including a distribution of one or more types of microbes on a body surface of the individual;
selecting one or more treatment agents from a list of available treatment agents to apply to the body surface to modulate the one or more types of microbes on the body surface of the individual; and
arranging for delivery of the selected one or more treatment agents.

168. The method of claim 167, further comprising:
comparing the information associated with the microbe profile of the individual with at least one reference microbe profile; and
selecting the one or more treatment agents from the list of available treatment agents based on the comparison of the information associated with the microbe profile of the individual with the at least one reference microbe profile.

169. The method of claim 167, wherein comparing the information associated with the microbe profile of the individual with the at least one reference microbe profile comprises comparing the information associated with the microbe profile of the individual with at least one historical microbe profile of the individual.

170. The method of claim 167, wherein comparing the information associated with the microbe profile of the individual with the at least one reference microbe profile comprises comparing the information associated with the microbe profile of the individual with at least one microbe profile of one or more other individuals.

171. The method of claim 167, wherein comparing the information associated with the microbe profile of the individual with the at least one reference microbe profile comprises comparing the information associated with the microbe profile of the individual with at least one theoretical microbe profile.

172. The method of claim 167, wherein receiving the information associated with the microbe profile of the individual from the remote source comprises automatically receiving the information associated with the microbe profile of the individual from the remote source.

173. The method of claim 167, wherein receiving the information associated with the microbe profile of the individual comprises receiving the information associated with the microbe profile of the individual with a computing device.

174. The method of claim 167, wherein receiving the information associated with the microbe profile of the individual from the remote source comprises receiving the information associated with the microbe profile of the individual from a remote computing device.

175. The method of claim 167, wherein receiving the information associated with the microbe profile of the individual from the remote source comprises receiving the information associated with the microbe profile of the individual from an analyzer of a microbe profiling kit.

176. The method of claim 167, wherein receiving the information associated with the microbe profile of the individual from the remote source comprises receiving the information associated with the microbe profile of the individual from a kiosk.

177. The method of claim 167, wherein receiving the information associated with the microbe profile of the individual from the remote source comprises receiving the information associated with the microbe profile of the individual from a microbe profiling device.

178. The method of claim 167, wherein selecting the one or more treatment agents from the list of available treatment agents comprises selecting at least one of a probiotic agent, a prebiotic agent, an antimicrobial agent, a therapeutic agent, an anti-aging agent, an antiseptic agent, an anesthetic agent, an anti-acne agent, a cosmetic agent, a moisturizing agent, an astringent agent, a sunscreen agent, or a retinoid agent.

179. The method of claim 167, wherein selecting the one or more treatment agents from a list of available treatment agents comprises selecting the one or more treatment agents from a list of available treatment agents stored in at least one dataset.

180. The method of claim 167, wherein selecting the one or more treatment agents from a list of available treatment agents comprises selecting the one or more treatment agents from a list of available treatment agents stored in at least one look-up table.

181. The method of claim 167, wherein selecting the one or more treatment agents from the list of available treatment agents to treat the one or more types of microbes on the body surface of the individual comprises selecting the one or more treatment agents from the list of available treatment agents to modulate the one or more types of microbes on a skin surface of the individual.

182. The method of claim 167, wherein selecting the one or more treatment agents from the list of available treatment agents to treat the one or more types of microbes on the body surface of the individual comprises selecting the one or more treatment agents from the list of available treatment agents to modulate the one or more types of microbes on a gastrointestinal surface of the individual.

183. The method of claim 167, wherein selecting the one or more treatment agents from the list of available treatment agents to treat the one or more types of microbes on the body surface of the individual comprises selecting the one or more treatment agents from the list of available treatment agents to modulate the one or more types of microbes on a mucosal surface, a vaginal surface, a nasal surface, or an oral surface of the individual.

184. The method of claim 167, further including selecting the one or more treatment agents from the list of available treatment agents based on the microbe profile of the individual and one or more other factors.

185. The method of claim 184, wherein selecting the one or more treatment agents from the list of available treatment agents based on the microbe profile of the individual and the one or more other factors comprises selecting the one or more treatment agents from the list of available treatment agents based on the microbe profile of the individual and one or more of age, gender, ethnicity, skin characteristics, geographical location, medical history, co-morbidities, or user preferences.

186. The method of claim 184, wherein selecting the one or more treatment agents from the list of available treatment agents comprises automatically selecting the one or more treatment agents from the list of available treatment agents.

187. The method of claim 167, further including receiving user information from the individual.

188. The method of claim 187, wherein receiving user information from the individual comprises receiving age, gender, ethnicity, skin characteristic, geographical location, medical history, co-morbidities, or user preferences from the individual.

189. The method of claim 167, wherein arranging for the delivery of the selected one or more treatment agents comprises automatically arranging for the delivery of the selected one or more treatment agents.

190. The method of claim 167, wherein arranging for the delivery of the selected one or more treatment agents comprises arranging for the delivery of the selected one or more treatment agents to a street address.

191. The method of claim 167, wherein arranging for the delivery of the selected one or more treatment agents comprises arranging for the delivery of the selected one or more treatment agents to a postal address.

192. The method of claim 167, wherein arranging for the delivery of the selected one or more treatment agents comprises arranging for the delivery of the selected one or more treatment agents to a residence, a post office, a delivery service office, a store, a pharmacy, a medical office, or a cosmetic counter.

193. The method of claim 167, further comprising:
notifying the individual that delivery of the selected one or more treatment agents has been arranged.

194. The method of claim 193, wherein notifying the individual comprises notifying the individual by at least one of an electronic communication, a telephonic communication, or a written communication.

195. A system, comprising:
circuitry for receiving information associated with a microbe profile of an individual from a remote source, the microbe profile including a distribution of one or more types of microbes on a body surface of the individual;
circuitry for selecting one or more treatment agents from a list of available treatment agents to apply to the body surface to modulate the one or more types of microbes on the body surface of the individual; and
circuitry for arranging for delivery of the selected one or more treatment agents.

196. The system of claim 195, further comprising:
a computing device.

197. The system of claim 195, further comprising:
circuitry for comparing the information associated with the microbe profile of the individual with at least one reference microbe profile; and
circuitry for selecting the one or more treatment agents from the list of available treatment agents based on the comparison of the information associated with the microbe profile of the individual with the at least one reference microbe profile.

198. The system of claim 197, wherein the circuitry for comparing the information associated with the microbe profile of the individual with the at least one reference microbe profile comprises circuitry for comparing the information associated with the microbe profile of the individual with at least one historical microbe profile of the individual.

199. The system of claim 197, wherein the circuitry for comparing the information associated with the microbe profile of the individual with the at least one reference microbe profile comprises circuitry for comparing the information associated with the microbe profile of the individual with at least one microbe profile of one or more other individuals.

200. The system of claim 197, wherein the circuitry for comparing the information associated with the microbe profile of the individual with the at least one reference microbe profile comprises circuitry for comparing the information associated with the microbe profile of the individual with at least one theoretical microbe profile.

201. The system of claim 195, wherein the circuitry for receiving the information associated with the microbe profile of the individual from the remote source comprises circuitry for automatically receiving the information associated with the microbe profile of the individual from the remote source.

202. The system of claim 195, wherein the circuitry for receiving the information associated with the microbe profile of the individual comprises circuitry for receiving the information associated with the microbe profile of the individual with a computing device.

203. The system of claim 195, wherein the circuitry for receiving the information associated with the microbe profile of the individual from the remote source comprises circuitry for receiving the information associated with the microbe profile of the individual from a remote computing device.

204. The system of claim 195, wherein the circuitry for receiving the information associated with the microbe profile of the individual from the remote source comprises circuitry for receiving the information associated with the microbe profile of the individual from an analyzer of a microbe profiling kit.

205. The system of claim 195, wherein the circuitry for receiving the information associated with the microbe profile of the individual from the remote source comprises circuitry for receiving the information associated with the microbe profile of the individual from a kiosk.

206. The system of claim 195, wherein the circuitry for receiving the information associated with the microbe profile of the individual from the remote source comprises circuitry for receiving the information associated with the microbe profile of the individual from a microbe profiling device.

207. The system of claim 195, wherein the circuitry for selecting the one or more treatment agents from the list of available treatment agents comprises circuitry for selecting at least one of a probiotic, a prebiotic, an antimicrobial, a therapeutic agent, an anti-aging agent, an antiseptic, an anesthetic agent, an anti-acne agent, a cosmetic agent, a moisturizing agent, an astringent agent, a sunscreen agent, or a retinoid agent.

208. The system of claim 195, wherein the circuitry for selecting the one or more treatment agents from a list of available treatment agents comprises circuitry for selecting one or more treatment agents from a list of available treatment agents stored in at least one dataset.

209. The system of claim 195, wherein the circuitry for selecting the one or more treatment agents from a list of available treatment agents comprises circuitry for selecting one or more treatment agents from a list of available treatment agents stored in at least one look-up table.

210. The system of claim 195, wherein the circuitry for selecting the one or more treatment agents from the list of available treatment agents to apply to the body surface to modulate the one or more types of microbes on the body surface of the individual comprises circuitry for selecting the one or more treatment agents from the list of available treatment agents to modulate the one or more types of microbes on a skin surface of the individual.

211. The system of claim 195, wherein the circuitry for selecting the one or more treatment agents from the list of available treatment agents to apply to the body surface to modulate the one or more types of microbes on the body surface of the individual comprises circuitry for selecting the one or more treatment agents from the list of available treatment agents to modulate the one or more types of microbes on a gastrointestinal surface of the individual.

212. The system of claim 195, wherein the circuitry for selecting the one or more treatment agents from the list of available treatment agents to apply to the body surface to modulate the one or more types of microbes on the body surface of the individual comprises circuitry for selecting the one or more treatment agents from the list of available treatment agents to modulate the one or more types of microbes on a mucosal surface, a vaginal surface, a nasal surface, or an oral surface of the individual.

213. The system of claim 195, further including circuitry for selecting the one or more treatment agents from the list of available treatment agents based on the microbe profile of the individual and one or more other factors.

214. The method of claim 213, wherein the circuitry for selecting the one or more treatment agents from the list of available treatment agents based on the microbe profile of the individual and the one or more other factors comprises circuitry for selecting the one or more treatment agents from the list of available treatment agents based on the microbe profile of the individual and one or more of age, gender, ethnicity, skin characteristics, geographical location, medical history, co-morbidities, or user preferences.

215. The system of claim 195, wherein the circuitry for selecting the one or more treatment agents from the list of available treatment agents comprises automatically selecting the one or more treatment agents from the list of available treatment agents.

216. The system of claim 195, further including circuitry for receiving user information from the individual.

217. The system of claim 216, wherein the circuitry for receiving the user information from the individual comprises circuitry for receiving age, gender, ethnicity, skin characteristics, geographical location, medical history, co-morbidities, or user preferences from the individual.

218. The system of claim 195, wherein the circuitry for arranging for the delivery of the selected one or more treatment agents comprises circuitry for automatically arranging for the delivery of the selected one or more treatment agents.

219. The system of claim 195, wherein the circuitry for arranging for the delivery of the selected one or more treatment agents comprises circuitry for arranging for the delivery of the selected one or more treatment agents to a street address.

220. The system of claim 195, wherein the circuitry for arranging for the delivery of the selected one or more treatment agents comprises circuitry for arranging for the delivery of the selected one or more treatment agents to a postal address.

221. The system of claim 195, wherein the circuitry for arranging for the delivery of the selected one or more treatment agents comprises circuitry for arranging for the delivery of the selected one or more treatment agents to a residence, a post office, a delivery service office, a store, a pharmacy, a medical office, or a cosmetic counter.

222. The system of claim 195, further comprising:
circuitry for arranging for delivery of one or more microbe sampling units.

223. The system of claim 195, further comprising:
circuitry for notifying the individual that delivery of the selected one or more treatment agents has been arranged.

224. The system of claim 223, wherein the circuitry for notifying the individual comprises circuitry for notifying the individual by at least one of an electronic communication, a telephonic communication, or a written communication.
